(19) United States
(12) Patent Application Publication
(10) Pub. No.: US 2019/0225652 A1
(43) Pub. Date: Jul. 25, 2019

(54) GENERATION OF PEPTIDES

(71) Applicants: Hexima Limited, La Trobe University, Victoria (AU); La Trobe University, Bundoora, Victoria (AU); The University of Queensland, Brisbane, Queensland (AU)

(72) Inventors: Karen Sandra Harris, Pascoe Vale South (AU); Marilyn Anne Anderson, Keilor (AU); Thomas Matthew Alcorn Shafee, Bellfield (AU); Thomas Durek, Auchenflower (AU); Mark Jackson, Karana Downs (AU); David Craik, Chapel Hill (AU)

(21) Appl. No.: 15/762,433

(22) PCT Filed: Sep. 23, 2016
(86) PCT No.: PCT/AU2016/050897

§ 371 Date: Mar. 22, 2018

(30) Foreign Application Priority Data
Sep. 25, 2015 (AU) 2015903918

(51) Int. Cl. C07K 7/64; C07K 1/02; C07K 1/107; C12N 15/86; C12N 9/50; G01N 33/542; C12P 21/02; C12N 15/52; C12N 15/70; C12N 15/81; A01N 63/02

(52) U.S. Cl. CPC C07K 7/64; C07K 1/02; C07K 1/1075; C12N 15/86; C12N 9/63; G01N 33/542; G01N 2333/95; C12N 15/52; C12N 15/70; C12N 15/81; A01N 63/02; C12Y 304/22034; C12P 21/02

(57) ABSTRACT

The present disclosure relates generally to generation of a recombinant enzyme with cyclization activity and its use for generating cyclic peptides as well as linear peptide conjugates.

### FILING DATA

This application is associated with and claims priority from Australian Provisional Patent Application No. 20159036918, filed on 25 Sep. 2015, entitled “Generation of peptides”, the entire contents of which, are incorporated herein by reference.

### BACKGROUND

### Field

The present disclosure relates generally to generation of a recombinant enzyme with cyclization activity and its use for generating cyclic peptides as well as linear peptide conjugates.

### Description of Related Art

Bibliographic details of the publications referred to by author in this specification are collected alphabetically at the end of the description.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavor to which this specification relates.

Proteases are abundant throughout nature and are essential for a wide range of cellular processes. They typically serve to hydrolyze polypeptide chains, resulting in either degradation of the target sequence or maturation to a biologically active form. Less frequently, proteases can act as ligases to link distinct polypeptides, producing new or alternately spliced variants. This unusual function has been reported for processes such as the maturation of the lectin, Concanavalin A (Sheldon et al. (1996) Biochem. J. 320:865-870), peptide presentation by major histocompatibility complex class I molecules (Hanada et al. (2004) Nature 427:252-256) and anchoring of bacterial proteins to the cell wall (Mazmanian et al. (1999) Science (80) 285:760-763). This enzymatic transpeptidation has also been implicated in the backbone-cyclization of ribosomally synthesized cyclic peptides (Barber et al. (2013) J. Biol. Chem. 288:12500-12510; Nguyen et al. (2014) Nat. Chem. Biol. 10:732-738; Luo et al. (2014) Chem. Biol. 1-8 doi:10.1016/j.chembiol.2014.10.015; Lee et al. (2009) J. Am. Chem. Soc. 131:2122-2124).

Gene-encoded cyclic peptides have been identified in a range of organisms including plants, fungi, bacteria and animals (Arnison et al. (2013) Nat Prod Rep 30:108-160). In plants, they are divided into four classes: cyclotides (e.g. the prototypical cyclotide kalata B1 [kB1]) (Gillon et al. (2008) Plant J. 53:505-515; Saska et al. (2007) J. Biol. Chem. 282:29721-29728), PawS-derived trypsin inhibitors (e.g. sunflower trypsin inhibitor (SFTI)) [Mylne et al. (2011) Nat. Chem. Biol. 7:257-259], knottin trypsin inhibitors (e.g. Momordica cochinchinensis trypsin inhibitor (MCoTI-II)) [Mylne et al. (2012) Plant Cell 24:2765-2778] and orbitides (e.g. segetalins) [Barber et al. (2013) supra].

Cyclotides were first identified in the African plant Oldenlandia affinis and exhibit insecticidal, nematocidal and molluscicidal activity against agricultural pests (Jennings et al. (2001) Proc. Natl. Acad. Sci. U.S.A 98:10614-10619; Plan et al. (2008) J. Agric. Food Chem. 56:5237-5241; Colgrave et al. (2008) Biochemistry 47:5581-5589; Colgrave et al. (2009) Acta Trop. 109:163-166). Other reported activities include neurotensin antagonism (Witherup et al. (1994) J. Nat. Prod 57:1619-1625), anti-HIV activity (Gustafson et al. (2000) J. Nat. Prod 63:176-178), anti-microbial activity (Tam et al. (1999) Proc. Natl. Acad. Sci. U.S.A 96:8913-8918), cytotoxic activity (Lindholm et al. (2002) Mol. Cancer Ther. 1:365-369), uterotonic activity (Gran (1973) Acta pharmacol. toxicol. 33:400-408), and hemolytic (Tam et al. (1999) supra) and anti-fouling properties (Goransson et al. (2004) J. Nat. Prod. 67:1287-1290). Cyclotides are characterized by a cystine knot motif that, together with backbone cyclization, confers exceptional stability. This has generated much interest in the cyclotide framework as a pharmaceutical scaffold; a potential heightened by the successful grafting of bioactive sequences into both Möbius and trypsin inhibitor cyclotides (Poth et al. (2013) Biopolymers 100:480-491). Backbone cyclization can also increase the stability and facilitate the oral administration route for bioactive linear peptides, suggesting that this modification will find broad application (Clark et al. (2005) Proc. Natl. Acad. Sci. United States Am. 102:13767-13772; Clark et al. (2010) Angew. Chem. Int. Ed. Engl. 49:6545-8; Chan et al. (2013) Chembiochem 14:617-624). Elucidating the mechanism of enzymatic cyclization intrinsic to cyclotide biosynthesis is important not only for the realization of the pharmaceutical and agricultural potential of cyclotides, but also for increasing the cyclization efficiency of unrelated, bioactive peptides.

Cyclotides are produced from precursor molecules in which the cyclotide sequence is typically flanked by N- and C-terminal propeptides. The first processing event is the removal of the N-terminal propeptide, producing a linear precursor that remains linked to the C-terminal prodomain (Gillon et al. (2008) supra). The final maturation step involves enzymatic cleavage of this C-terminal region and subsequent ligation of the free C- and N-termini. However, only four native cyclases have been identified to date (Barber et al. (2013) supra; Nguyen et al. (2014) supra; Luo et al. (2014) supra; Lee et al. (2009) supra; Gillon et al. (2008) supra). The best characterized of these is the serine protease PatG, which is responsible for maturation of the bacterial cyanobactins (Lee et al. (2009) supra). In plants, the serine protease PCY1 reportedly facilitates cyclization of the segetalins, cyclic peptides from the Caryophyllaceae (Barber et al. (2013) supra). In the other three classes of plant-derived cyclic peptides, strong Asx sequence (where x is N (asparagine) or D (aspartic acid)) conservation at the P1 residue of the C-terminal cleavage site suggested involvement of a group of cysteine proteases known as vacuolar processing enzymes (VPEs) or asparaginyl endopeptidases (AEPs) in this process (Gillon et al. (2008) supra).

Of the small number of AEPs which have been demonstrated to preferentially act as peptide ligases, only one of these, butelase 1, has been shown to be an efficient cyclase (Bernath-Levin et al. (2015) Chemistry & Biology 22:1-12; Nguyen et al. (2014) supra; Sheldon et al. (1996) supra). The structural basis for the preferential ligase activity of this subset of AEPs remains unknown.

Butelase-1 was isolated from the cyclotide producing plant Clitoria ternatea and shown to cyclize a modified precursor of kB1 from O. affinis, confirming the ability of this group of enzymes to mediate cyclization in vitro (Nguyen et al. (2014) supra) provided that the appropriate recognition sequences are added to the ends of the polypeptide precursor to be cyclized. However, recombinant butelase-1 from E. coli was only expressed in insoluble form and thus unable to mediate cyclization. Only one AEP with any cyclizing ability has been produced recombinantly, and this was highly inefficient, producing mainly hydrolyzed substrate (Bernath-Levin et al. (2015) supra). There is a need to develop methodology to generate a functional recombinant AEP so that it can be used to more efficiently generate cyclic peptides from polypeptide precursors as well as linear peptide conjugates.

### SUMMARY

The present disclosure teaches the production of a functional recombinant asparaginyl endopeptidase (AEP) and its use in an efficient method for producing a cyclic peptide or linear peptide conjugate. The term “cyclic peptide” includes but is not limited to a cyclotide. The cyclic peptide may be naturally cyclical or may be artificially cyclized to confer, for example, added stability, efficacy or utility. A linear peptide conjugate is the ligation of two or more peptides together in linear sequence. The term “peptide” is not to exclude a polypeptide or protein. For brevity, the term “peptide” is used to avoid any doubt, the present invention covers a cyclic peptide, cyclic polypeptide and cyclic protein as well as a linear peptide, linear polypeptide or linear protein. All encompassed by the term “cyclic peptide” or “linear peptide”.

The cyclic peptide or linear peptide can be used in a variety of applications relevant to human and non-human animals and plants. Included are agricultural applications such as the generation of topical agents for treatment of infection or infestation by a pathogen and pharmacological applications such as the treatment of cancer, cardiovascular disease, infectious disease, immune diseases and pain. Therapeutic agents may be delivered topically or systemically. In addition, both naturally cyclic peptides in linear form and naturally linear peptides can be subject to cyclization as well as linear polypeptide precursors comprising non-naturally occurring amino acids and/or modified side chains or modified cross-linkage bonds. The cyclization of a naturally linear peptide can lead inter alia to a longer half life and/or increased stability and/or the ability to be orally administered.

The cyclization process may be conducted in various ways and can employ prokaryotic or eukaryotic organisms and can act on a polypeptide precursor containing a non-naturally occurring amino acid residue or other modification. In essence, an asparaginyl endopeptidase (AEP) with cyclizing ability is employed to cyclize a linear polypeptide precursor or ligate together peptides including polypeptides and proteins. The term “polypeptide” includes a “protein”. The polypeptide precursor includes a precursor to a naturally cyclic peptide as well as a polypeptide which is naturally linear and is converted into a cyclic peptide.

The linear polypeptide precursor comprises a C-terminal AEP processing site. Generally, but not exclusively, the C-terminal processing site is an amino acid sequence defined as comprising P3 to P1 prior to the actual cleavage site and comprising P1′ to P3″ after the cleavage site towards the C-terminal end. In an embodiment, P3 to P1 and P1′ to P3′ have the amino acid sequence:

X₂X₃X₄X₅X₆X₇

wherein X is an amino acid residue and:

X₂ is optional or is any amino acid;

X₃ is optional or is any amino acid;

X₄ is Nor D;

X₅ is G or S;

X₆ is L or A or I; and

X₇ is optional or any amino acid.

In an embodiment, X₁ through X₆ comprise the amino acid sequence:

X₂X₃NGLX₇

wherein X₂, X₃ and X₇ are as defined above.

The N-terminal end of the linear polypeptide precursor may contain no specific AEP processing site or may contain a processing site defined by any one of P1′ through P3″ wherein P1 to P3″ is defined by:

X₉X₁₀X₁₁

wherein X is an amino acid residue:

X₉ is optional and any amino acid or G, Q, K, V or L;

X₁₀ is optional or any amino acid or L, F or I or an hydrophobic amino acid residue;

X₁₁ is optional and any amino acid.

In an embodiment, X₉ through X₁₁ comprise the amino acid sequence:

GLX₁₁

wherein X₁₁ is defined as above.

In an embodiment, the AEP processing site comprises N- and C-terminal end sequences comprising the sequence:

GLX₁₁ [Xₙ]X₂X₃NGLX₇

wherein X₁₁, X₂, X₃, and X₇ are as defined above and [Xₙ] is absent (n=0) or any amino acid residue in a sequence of from 1 to 2000 amino acids.

In an embodiment, the C-terminal processing site comprises P4 to P1 and P1′ to P4′ wherein P1 to P4 and P1′ to P4′ comprise X₁X₂X₃X₄X₅X₆X₇X₈ wherein X₂ to X₇ are as defined above and X₁ is optional or any amino acid and X₈ is optional or any amino acid.

In the case of a prokaryotic system, the AEP is produced in the cell and isolated before it is used in vitro with a linear polypeptide precursor to be cyclized. The linear polypeptide precursor may also be produced in the cell then separated or otherwise isolated from the cell and cyclized in vitro using the recombinant AEP. A polypeptide precursor produced by synthesis, including polypeptides with non-naturally occurring amino acids or a recombinant polypeptide with post-translational modification can also be cyclized in vitro using a recombinant AEP. The AEP and polypeptide precursor may also be co-expressed in a compartment of a prokaryotic cell such as but not limited to the periplasmic space. In which case, the resulting cyclic peptide is isolated from the cell.

A similar protocol is adapted when a eukaryotic organism is employed, such as a yeast (e.g. Pichia sp., Saccharomyces sp. or Kluyveromyces sp.). Genetic material encoding AEP is expressed enabling generation of cyclic peptides in vitro from a precursor polypeptide or in vivo if both the AEP and polypeptide are co-expressed. In either event, the resulting cyclic peptide is subject to isolation and purification from a vacuole or other cellular compartment in the eukaryotic cell or from the reaction vessel. Alternatively, the AEP and polypeptide precursor are produced in separate eukaryotic cells or in different compartments within the same cell, extracted and then co-incubated in vitro to generate the cyclic peptide. In yet another aspect only one or other of the AEP or polypeptide precursor is produced in the eukaryotic cell; the other component is supplied from a different source and the two are then incubated in vitro to generate a cyclic peptide.

Just to re-emphasize, the term “peptide” includes a polypeptide or protein as well as a peptide.

Enabled herein is a method for producing a cyclic peptide, the method comprising introducing into the prokaryotic or eukaryotic cell genetic material which, when expressed, generates a recombinant AEP with cyclization ability, isolating the AEP and incubating the AEP with a linear polypeptide precursor optionally modified to introduce a non-naturally occurring amino acid, the incubation being for a time and under conditions sufficient to generate a cyclic peptide from the polypeptide precursor. Alternatively, genetic material encoding the AEP with cyclization ability is co-expressed with genetic material encoding a linear polypeptide precursor in a cell for a time and under conditions sufficient to generate a cyclic peptide in a vacuole or other cellular compartment of the cell. This process can also occur in a membranous compartment of a prokaryotic cell such as a periplasmic space. In addition, the AEP can catalyze a ligation reaction to conjugate two or more peptides wherein at least one peptide comprises a C-terminal AEP recognition amino acid sequence and another peptide comprises an N-terminal AEP recognition amino acid sequence. The eukaryotic cell can also be used to generate one or both of the AEP and/or polypeptide precursor for use in the generation of a cyclic peptide in vitro. A cyclic peptide can also be produced in the prokaryotic cell. In an embodiment, the cyclic peptide is produced in the periplasmic space of a prokaryotic cell. As indicated above, reference to “peptide” includes a polypeptide or protein. No limitation in size or type of proteinaceous molecule is intended by use of the term “peptide”, “polypeptide” or “protein”.

In an embodiment, a linear peptide is generated using ligase activity of an AEP. In this embodiment, a first peptide comprising the C-terminal AEP recognition sequence is co-incubated with a second peptide comprising an N-terminal AEP recognition sequence which may or may not have a tag and an AEP. The AEP catalyses a ligation between the first and second peptides to generate a linear peptide conjugate. This may then subsequently be cyclized into a cyclic peptide or used as a linear peptide.

In an embodiment, regardless of the manner the cyclic peptide or peptide conjugate is generated, it is subject to isolation which includes purification.

Enabled herein is a method for producing a cyclic peptide, the method comprising co-incubating an AEP with peptide cyclization activity with a linear polypeptide precursor of the cyclic peptide for a time and under conditions sufficient to generate the cyclic peptide. Reference to “cyclic peptide” includes a “cyclotide”. By “co-incubation” means either in vitro in a reaction vessel or in a cell or in a compartment of a cell. Multiple peptides or repeat forms of the same peptides may also be cyclized in vitro or in vivo. Again, it is emphasized that the term “peptide” includes a polypeptide and a protein.

Hence, taught herein the AEP is generated in a prokaryotic cell or eukaryotic cell and used in vitro or in vivo to generate a cyclic peptide from a linear polypeptide precursor. The AEP and linear polypeptide precursor may also be co-expressed in a prokaryotic cell or eukaryotic cell. Alternatively, the linear polypeptide precursor may be produced by synthetic chemistry. In an embodiment, a recombinant AEP is produced in a prokaryotic or eukaryotic cell, isolated from the cell and used in vitro on any polypeptide precursor to generate a cyclic peptide.

Generally, the genetic material comprises nucleic acid which may be expressed in two respective nucleic acid constructs. Alternatively, the recombinant nucleic acid encoding each of the AEP and the polypeptide precursor is expressed in a single nucleic acid construct. Multiple repeats of the same peptide or of different peptides may also be subject to cyclization processing in vivo or in vitro. Notwithstanding, a key aspect is the production of a recombinant form of AEP which is functional having peptide cyclization activity which can either be used in vitro with a precursor polypeptide or a cell expressing an AEP can be used as a recipient for a genetic molecule encoding the precursor polypeptide.

Enabled herein is a set of rules to enable prediction of whether an AEP is a cyclase. The set of rules is based inter alia on the presence or absence of residues or gaps in at least 25% of 17 predictive sites. This equates to 5 or more. The sites encompass an activity preference loop (APL), active sites and sites proximal thereto and non-active surface residues. Predictive sites are summarized in Table 2. Hence, taught herein is a method for determining whether an AEP is likely to have cyclization activity, the method comprising determining the amino acid sequence of the AEP, aligning the sequence with a best fit to the amino acid sequence of OaAEP1_{b} (SEQ ID NO:1) and screening for the presence of 5 or more of residues or absence of residues at 139K, 161D, 186K, 192D, 247C, 248Y, 253Q, 255A, 263V, 293H, Gap, Gap, Gap, Gap, Gap (between residues 299 and 300), 314E and 316G wherein Gap means the absence of a residue wherein the presence of 5 or more of the listed residues or absence of residues is indicative of an AEP which is a cyclase. Further enabled herein is a method for determining whether an AEP is unlikely to have cyclization activity, the method comprising determining the amino acid sequence of the AEP, aligning the sequence with a best fit to the amino acid sequence of OaAEP1b (SEQ ID NO:1) and screening for the presence of 13 or more of the residues 139D, 161N, 186G, 192N, 247G, 248T, 253E, 255P, 263T, 293L, residues aligning between residues 299 and 300 of OaAEP1_{b}—N, G, N, Y and S, 314K and 316K wherein the presence of 13 or more of the listed residues is indicative of an AEP which is not a cyclase. The AEP may, therefore, be from any source such as but not limited to from the genus Oldenlandia. The AEP can be readily tested for cyclase activity. One such species is Oldenlandia affinis. Examples include OaAEP1b (SEQ ID NO:1), OaAEP1 (SEQ ID NO:2), OaAEP3 (SEQ ID NO:4) or a variant, derivative or hybrid form thereof which retains cyclizing activity. In an embodiment, the AEP has an amino acid sequence having at least 80% similarity to any one of SEQ ID NO:1, SEQ ID NO:2 or SEQ ID NO:4 after optimal alignment and wherein the AEP comprises the presence of 5 or more of residues or absence of residues at 139K, 161D, 186K, 192D, 247C, 248Y, 253Q, 255A, 263V, 293H, Gap, Gap, Gap, Gap, Gap (between residues 299 and 300), 314E and 316G wherein Gap means the absence of a residue when optimally aligned to SEQ ID NO:1. An example of a non-cyclase AEP excluded as a cyclase under this definition is OaAEP2 (SEQ ID NO:3). It is a proviso that statements encompassing cyclase AEPs do not include OaAEP2 (SEQ ID NO:3).

When the linear precursor is produced in a prokaryotic cell the first N-terminal residue in the construct is necessarily methionine. In the event that an N-terminal methionine precludes cyclization, alternative approaches are utilized. For example:

The endogenous methionine amino peptidase expressed by some E. coli strains is harnessed to remove the initiating methionine in vivo, revealing an N-terminus appropriate for cyclization (Camarero et al. (2001) Bioorganic Med Chem 9:2479-2484).

A recognition sequence for a protease that cleanly releases the additional residues (e.g. TEV protease, Factor Xa) is added N-terminal to a polypeptide precursor, exposing an appropriate N-terminus for cyclization following cleavage.

In an embodiment, the cyclic peptide has one of a number of activities such as exhibiting pharmaceutical activity and includes an antipathogenic, therapeutic or uterotonic property. Examples of therapeutic activities include anticancer, protease inhibitory, antiviral or immunomodulatory activity and the treatment of pain. The cyclic peptide may also comprise a functional portion fused or embedded in a backbone framework of a cyclotide or other cyclic scaffold (Poth et al. (2013) supra). The cyclic peptide may also be generated to be topically applied to a plant or seed of a plant to protect it from pathogen infection or infestation such as against a fungus, bacterium, nematode, mollusc, helminth, virus or protozoan organism. Alternatively, it is topically applied to human or non-human animal surfaces such as a nail, hair or skin. The polypeptide precursor may be a natural precursor for the generation of a cyclic peptide or it may not naturally become cyclic but is adapted to generate a cyclic peptide. Such a non-naturally occurring cyclic peptide may, for example, have a longer half life in a composition or when used in vivo or may have greater stability efficacy or utility.

Further enabled herein is a kit comprising an AEP and a receptacle adapted to receive a polypeptide precursor and means to admix the AEP with the polypeptide precursor. Reagents may also be included to facilitate conversion of the polypeptide precursor into a cyclic peptide. Alternatively, the kit contains a eukaryotic or prokaryotic cell comprising genetic material encoding an AEP. Further genetic material encoding a polypeptide precursor to be cyclized is then introduced to that cell. An example is a yeast cell such as a Pichia sp.

The kit enables a useful business model for generating cyclic peptides from any linear polypeptide precursor.

A summary of sequence identifiers used throughout the subject specification is provided in Table 1.

TABLE 1
Summary of sequence identifiers

| SEQUENCE | |
|---|---|
| ID NO: | DESCRIPTION |
| 1 | Oldenlandia affinis OaAEP1_{b} |
| 2 | Oldenlandia affinis OaAEP1 |
| 3 | Oldenlandia affinis OaAEP2 |
| 4 | Oldenlandia affinis OaAEP3 |
| 5 | Amino acid sequence of model peptide R1 |
| 6 | OaAEPdegen-F, 5′ forward primer |
| 7 | OaAEP1-R, 5′ reverse primer |
| 8 | OaAEP2-R, 5′ reverse primer |
| 9 | OaAEP3-R, 5′ reverse primer |
| 10 | C-terminal pro-hepta-peptide |
| 11 | Kalata B1mature + CTPP protein sequence |
| 12 | C-terminal flanking sequence for target peptide |
| 13 | Ligation partner |
| 14 | Ligation partner |
| 15 | Ligation product |
| 16 | Ligation product |
| 17 | Linker |
| 18 | Linker |
| 19 | Leaving group |
| 20 | 6xHis-ubiquitin-OaAEP1_{b} fusion protein |
| 21 | Internally quenched fluorescence peptide wt |
| 22 | Nucleotide sequence encoding kalata B1 precursor protein |
| 23 | Amino acid sequence of kalata B1 precursor protein |
| 24 | Amino acid sequence of model peptide Bac2A |
| 25 | Internally quenched fluorescence peptide L31A |
| 26 | R1 peptide derivative |
| 27 | R1 peptide derivative |
| 28 | R1 peptide derivative |
| 29 | R1 peptide derivative |
| 30 | R1 peptide derivative |
| 31 | R1 peptide derivative |
| 32 | C-terminal AEP recognition sequence |
| 33 | N-terminal AEP recognition sequence |
| 34 | C-terminal AEP recognition sequence |
| 35 | OaAEP1b nucleic acid sequence |
| 36 | OaAEP1 na seq |
| 37 | OaAEP2 na seq |
| 38 | OaAEP3 na seq |
| 39 | OaAEP4 aa seq from transcriptomics |
| 40 | OaAEP4 na seq codon optimized for E. coli expression |
| 41 | OaAEP5 aa seq from transcriptomics |
| 42 | OaAEP5 na seq codon optimized for E. coli expression |
| 43 | OaAEP6 aa seq from transcriptomics |
| 44 | OaAEP7 aa seq from transcriptomics |
| 45 | OaAEP8 aa seq from transcriptomics |
| 46 | OaAEP9 aa seq from transcriptomics |
| 47 | OaAEP10 aa seq from transcriptomics |
| 48 | OaAEP11 aa seq from transcriptomics |
| 49 | OaAEP12 aa seq from transcriptomics |
| 50 | OaAEP13 aa seq from transcriptomics |
| 51 | OaAEP14 aa seq from transcriptomics |
| 52 | OaAEP15 aa seq from transcriptomics |
| 53 | OaAEP16 aa seq from transcriptomics |
| 54 | OaAEP17 aa seq from transcriptomics |
| 55 | Nicotiana tabacum NtAEPlb |
| 56 | Petunia hybrida PxAEP3a |
| 57 | Petunia hybrida PxAEP3b |
| 58 | Clitoria ternatea CtAEP1 |
| 59 | Clitoria ternatea CtAEP2 |
| 60 | EcAMP1 peptide derivative |
| 61 | R1 peptide derivative |
| 62 | R1 peptide derivative |
| 63 | R1 peptide derivative |
| 64 | R1 peptide derivative |
| 65 | R1 peptide derivative |
| 66 | R1 peptide derivative |
| 67 | R1 peptide derivative |
| 68 | R1 peptide derivative |
| 69 | R1 peptide derivative |
| 70 | R1 peptide derivative |
| 71 | SFTI-I10R peptide product |
| 72 | SFTI-I10R peptide + Ubiquitin + His tag |
| 73 | Kalata B1 peptide product |
| 74 | Kalata B1 + Ubiquitin + His tag |
| 75 | Vc1.1 peptide + linker product |
| 76 | Vc1.1 + linker + Ubiquitin + His tag |
| 77 | Kalata B1 + OaAEP1b aa seq |
| 78 | Kalata B1 + OaAEP1b na seq codon optimized |
| 79 | Target peptide |
| 80 | Ligation partner peptide |
| 81 | Ligated peptide product |
| 82 | Ligated peptide product |
| 83 | Target peptide |
| 84 | Ligated peptide product + C-terminal biotin |
| 85 | Ligated peptide product + N-terminal biotin |
| 86 | R1 peptide derivative |
| 87 | Bac2A derivative |
| 88 | Kalata B1 derivative |
| 89 | R1 peptide derivative |
| 90 | R1 peptide derivative |
| 91 | R1 peptide derivative |
| 92 | Cicer arietinum |
| 93 | Medicago truncatula |
| 94 | Hordeum vulgare |
| 95 | Gossypium raimondii |
| 96 | Chenopodium quinoa |
| 97 | CtAEP6 |
| 98 | NaD1 |
| 99 | Ligated peptide |
| 100 | Ligated peptide |
| 101 | R1 peptide derivative |
| 102 | Ligation peptide |
| 103 | R1 peptide derivative |
| 104 | Ligated peptide |

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIG. 1A is a schematic representation of the Oak1 gene product. The precursor protein encoded by the Oak1 gene (SEQ ID NO:23 encoded by SEQ ID NO:22) is proteolytically processed to produce mature kB1. The domains shown in order are: ER signal peptide (ER SP), N-terminal propeptide (NTPP), N-terminal repeat (NTR), cyclotide domain, C-terminal propeptide (CTPP). Dashed lines indicate the N- and C-terminal processing sites and a bold asterisk denotes the rOaAEP1_{b} cleavage site. The C-terminal P3-P1 and P1′-P3′ sites are indicated. P1″-P3″ denote the N-terminal residues that replace the P1′-P3′ residues upon release of the C-terminal propeptide and subsequent backbone cyclisation. FIG. 1B is a schematic representation of a synthetic kalata B1 precursor carrying the native C-terminal pro-hepta-peptide (GLPSLAA—SEQ ID NO:10).

FIGS. 2 A and B is a Clustal Omega (Sievers et al. (2011) Mol. Syst. Biol 7: 539) alignment of the full-length protein sequences of OaAEP1b, OaAEP3, OaAEP4 and OaAEP5.

FIG. 3 is a graphical representation showing expression of active rOaAEP1_{b} in E. coli. (A) Pooled rOaAEP1b-containing anion exchange fractions pre- and post-activation at low pH were diluted 1:14 and tested for activity against the wildtype internally quenched fluorescence (wtIQF) peptide (11 μM) [SEQ ID NO:21]. Baseline fluorescence from a no-substrate control has been subtracted and the relative fluorescence intensity (RFU) at t=90 minutes is reported. A single representative experiment of two technical replicates is shown. (B) Activated rOaAEP1_{b} was captured by cation exchange and the final product analyzed by SDS-PAGE followed by (i) Instant blue staining and (ii) Western blotting with anti-AEP1_{b} polyclonal rabbit serum.

FIG. 4 is a representation of the amino acid sequence encoded by the OaAEP1b gene isolated from O. affinis genomic DNA (SEQ ID NO:1). Predicted ER signal sequence shown in grey; N-terminal propeptide shown in italics; the putative signal peptidase cleavage site is indicated by an open triangle and autocatalytic processing sites are indicated by filled triangles. The mature OaAEP1b cyclase domain is underlined. Cys217 and His175, presumed to be important for catalytic activity, are shown in bold and labeled with an asterisk. The dotted underline indicates possible processing sites for generation of the mature enzyme.

FIG. 5A shows an alignment of the sequence region containing the activity preference loop (APL) for three AEP sequences which act preferentially as proteases (NtAEP1b (SEQ ID NO:55), PxAEP3a (SEQ ID NO:56) and OaAEP2 (SEQ ID NO:3)) and two which act preferentially as cyclases (PxAEP3b (SEQ ID NO:57) and OaAEP1b (SEQ ID NO:1). FIG. 5B shows an alignment of OaAEP1b (preferentially a cyclase) and OaAEP2 (preferentially a protease) indicating the positions of the 17 cyclase predictive residues (or sites).

FIG. 6 is a graphical representation showing the MALDI MS profile of the enzymatic processing products of a linear kB1 precursor (kB1_{wt}) containing the C-terminal propeptide in the presence of rOaAEP1_{b}. Pre, linear precursor; Cyc, cyclic product. The +6 Da peak corresponds to the reduced form of the cyclic product.

FIG. 7 is a graphical representation showing the kinetics of rOaAEP1_{b}-mediated cyclisation. Varying concentrations of substrate (kB1_{wt} precursor) were incubated with enzyme (19.7 μg mL⁻¹ total protein) for 5 min. The amount of product formed was inferred by monitoring depletion of the precursor by RP-HPLC. A Michaelis-Menten plot shows the mean of three technical replicates and error bars report the standard error of the mean (SEM). The kinetic parameters derived from this plot are listed (±SEM).

FIG. 8A is a graphical representation of the cyclization by rOaAEP1_{b} (12 μg mL⁻¹ total protein) of Bac2A (RLARIVVIRVAR—SEQ ID NO:24), a linear peptide derivative of bactenecin. The product was analysed by MALDI MS 22 hours post-addition of rOaAEP1_{b} (+ enzyme) or water (− enzyme). Bold residues, added flanking enzyme recognition sequences. Asterisk, rOaAEP1_{b} cleavage site. Observed monoisotopic masses (Da; [M+H]⁺) are listed. +22 Da peaks likely represent Na⁺ adducts. Cyc, cyclic product; Pre, linear precursor. FIG. 8B is a graphical representation showing the MALDI MS profile of the enzymatic processing products of target peptides with additional AEP recognition residues after 5 h. The target peptides shown are (A) the R1 variant GLPVFAEFLPLFSKFGSRMHILKSTRNGL (SEQ ID NO:86), and (B) the Bac2A variant GLPRLARIVVIRVARTRNGLP (SEQ ID NO:87) with bold residues indicating additional AEP residues. The enzymes used were (i) rOaAEP1_{b}, (ii) rOaAEP3, (iii) rOaAEP4 and (iv) rOaAEP5 and all were at a final concentration of 19.7 μg mL⁻¹ total protein. A no enzyme control (v) is also shown. The expected monoisotopic mass of the cyclized variants are 3074.7 and 2042.3 Da [M+H]⁺ for the R1 variant and the Bac2A variant respectively. The observed monoisotopic masses are listed in the figure (Da; [M+H]⁺]). The +22 Da peak likely represents a sodium adduct.

FIG. 9 is a graphical representation showing the ESI MS profile of the enzymatic processing products of EcAMP1 with additional AEP recognition residues (GLPGSGRGSCRSQCMRRHEDEPWRVQECVSQCRRRRGGGDTRNGLP (SEQ ID NO:60), bold residues indicate additional AEP recognition residues) after 5 h. The enzymes used were (i) rOaAEP1_{b}, (ii) rOaAEP3, (iii) rOaAEP4 and (iv) rOaAEP5 and all were at a final concentration of 19.7 μg mL⁻¹ total protein. A no enzyme control (v) is also shown. The expected monoisotopic mass of cyclic EcAMP1 is 4892.3 Da. The observed monoisotopic masses are listed in the figure (Da).

FIG. 10 is a graphical representation of the cyclisation of the R1 model peptide with various flanking sequences by bacterially expressed, recombinant AEPs. The proportion of cyclic product is displayed after cyclisation by (A) OaAEP1_{b} (1 h incubation), (B) OaAEP3 (5 h incubation), (C) OaAEP4 (5 hr incubation) or (D) OaAEP5 (1 h incubation). In all cases, the enzyme was added at a final concentration of 19.7 μg mL⁻¹ total protein. --- represents the model peptide, R1 (VFAEFLPLFSKFGSRMHILK) and additional flanking residues are as indicated R1 Peptides: GLP---STRGLP (SEQ ID NO:26), GL---NGL (SEQ ID NO:27), GL---NG (SEQ ID NO:28), ---NGL (SEQ ID NO:29), GL---GHV (SEQ ID NO:61), GL---NHV (SEQ ID NO:62), GL---NHL (SEQ ID NO:63), GL---NGH (SEQ ID NO:64), GL---NGF (SEQ ID NO:65), GL---NFL (SEQ ID NO:66), GL---DGL (SEQ ID NO:67), LL---NGL (SEQ ID NO:89), QL---NGL (SEQ ID NO:30), KL---NGL (SEQ ID NO:31), GK---NGL (SEQ ID NO:90), GF---NGL (SEQ ID NO:91). The average of three technical replicates are shown and the error bars report the standard error of the mean (SEM).

FIG. 11 is a schematic representation of polypeptide ligation catalyzed by AEPs between a target peptide and a ligation partner peptide. The AEP cleavage site is indicated by ▾. For C-terminal labelling, an AEP cleavage site is incorporated into the target peptide and the ligation partner peptide contains an AEP-compatible N-terminus. For N-terminal labelling, an AEP cleavage site is incorporated into the ligation partner peptide and the target peptide contains an AEP-compatible N-terminus. AEP recognition residues added to the target peptides are shown in bold and the leaving groups are underlined.

FIG. 12 is a graphical representation showing the ESI MS profile of the enzymatic processing products of a target peptide (140 μM; GLP-NaD1-TRNGLP (SEQ ID NO:79)) and ligation partner peptides (700 μM) after 6-22 h, as indicated. The enzymes used were (A) rOaAEP1_{b}, (B) rOaAEP3, (C) rOaAEP4 and (D) rOaAEP5 and all were at a final concentration of 19.7 μg mL⁻¹ total protein. In panel (i) the ligation partner was GLPVSGE (SEQ ID NO:14). In panel (ii) the ligation partner was PLPVSGE (SEQ ID NO:80). In panel (iii) no ligation partner was added. The labelled NaD1 product has the ligation partner peptide added to the C-terminus. The expected monoisotopic mass of labelled NaD1 is 6641.3 Da when the ligation partner is GLPVSGE and 6681.3 Da when the ligation partner is PLPVSGE. The observed monoisotopic masses are listed in the figure (Da).

FIG. 13 is a graphical representation showing the MALDI MS profile of the enzymatic processing products of a target peptide (140 μM; R1 variant GKVFAEFLPLFSKFGSRMHILKNGL (SEQ ID NO:90)) and a ligation partner peptide (700 μM; GLK-biotin) after 6 h. The enzymes used were (A) rOaAEP1_{b}, (B) rOaAEP3, (C) rOaAEP4 and (D) rOaAEP5 and all were at a final concentration of 19.7 μg mL⁻¹ total protein. In panel (i) the ligation partner peptide was added. In panel (ii) no ligation partner peptide was added. The ligated product has a C-terminal biotin. The expected average mass of the biotin labelled product is 3192.9 Da [M+H]⁺ and the observed average masses are listed in the figure (Da; [M+H]⁺]).

FIG. 14 is a graphical representation showing the MALDI MS profile of the enzymatic processing products of a target peptide (140 μM; R1 variant GLVFAEFLPLFSKFGSRMHILKGHV (SEQ ID NO:61)) and a ligation partner peptide (700 μM; biotin-TRNGL) after 6 h. The enzymes used were (A) rOaAEP1_{b}, (B) rOaAEP3, (C) rOaAEP4 and (D) rOaAEP5 and all were at a final concentration of 19.7 μg mL⁻¹ total protein. In panel (i) the ligation partner peptide was added. In panel (ii) no ligation partner peptide was added. The +22 Da peak is likely a sodium adduct. The ligated product has an N-terminal biotin. The expected average mass of the biotin labelled product is 3430.1 Da [M+H]⁺ and the observed average masses are listed in the figure (Da; [M+H]⁺]).

FIG. 15 is a graphical representation of the activity of recombinant O. affinis AEPs (˜5 μg mL⁻¹ total protein) and rhuLEG (1 μg mL⁻¹ total protein) over time against the fluorogenic substrate Z-AAN-MCA (100 μM). Activity is tracked at 1 minute intervals at 37° C. for 60 minutes using excitation and emission wavelengths of 360 and 460 nm respectively. A single representative experiment is shown. RFU, relative fluorescence units.

FIG. 16 is a graphical representation of rOaAEP1_{b} activity against the IQF peptide Abz-STRNGLPS-Y(3NO₂) [SEQ ID NO:21] in the presence of protease inhibitors. rOaAEP1_{b} (4.4 μg mL⁻¹ total protein) was allowed to cleave the IQF peptide (11 μM) for 90 minutes. Enzyme activity against the IQF peptide in the presence of either the Ac-YVAD-CHO or Ac-STRN-CHO inhibitors is reported relative to a no inhibitor control at the 90 minutes time point.

FIGS. 17A and 17B are graphical representations of substrate specificity of plant and human AEPs for wt (SEQ ID NO:21) and L31A (SEQ ID NO:25) IQF peptide substrates. Initial velocity of recombinant O. affinis AEPs (˜10 μg mL⁻¹ total protein) (17A) and rhuLEG (1.1 μg mL⁻¹ total protein) (17B) against 50 μM IQF peptide substrates is shown. The assay was conducted at 37° C. The average of two technical replicates are shown and the error bars report the range.

FIG. 18A is a diagrammatic representation of a cyclotide construct for expression in E. coli comprising a cyclotide domain joined via a short linker to ubiquitin-6xHis. Filled triangle, AEP cleavage site. FIG. 18B is a diagrammatic representation of an alternative cyclotide construct for expression in E. coli comprising a methionine followed by the kalata B1 N-terminal repeat (NTR), cyclotide domain, short linker and ubiquitin-6xHis.

FIG. 19A is a graphical representation showing the MALDI MS profile of the enzymatic processing products of target peptides fused to ubiquitin. The target peptides are (A) SFTI1-I10R-ubiquitin (SEQ ID NO:72) (1 mg mL⁻¹ total protein), (B) kB1-ubiquitin (SEQ ID NO:74) (0.9 mg mL⁻¹ total protein) and (C) Vc1.1-ubiquitin (SEQ ID NO:76) (0.24 mg mL⁻¹ total protein). The masses produced after incubation for 22 h with (i) rOaAEP1_{b} (19.7-98.5 μg mL⁻¹), (ii) rOaAEP4 (19.7-30 μg mL⁻¹) or (iii) no enzyme are shown. Cyc denotes cyclic product. The +22 Da peak is likely a sodium adduct, the −16 Da peak is likely oxidized methionine, the +60 Da peak is likely cyclic product carrying both sodium (+22 Da) and potassium (+38 Da) adducts or may derive from an impurity in the preparation. FIG. 19B is a graphical representation showing enzymatic processing of the kalata B1-ubiquitin fusion protein (SEQ ID NO:74) (260 μg mL⁻¹ total protein) by different AEPs (19.7 μg mL⁻¹ total protein) after a 22 h incubation. Approximately 2 μg of starting material was analysed by SDS-PAGE followed by Western blotting with an anti-6xHis mouse monoclonal antibody.

FIG. 20A is a diagrammatic representation of constructs for Pichia pastoris transformation. Construct 1 contains the elements in a single construct and comprises, in sequence, an ER signal sequence, a vacuolar targeting signal (Vac), a cyclotide domain, a short linker and a pro-AEP domain. Construct 2 comprises an ER signal sequence, a vacuolar targeting signal, a cyclotide domain and a short linker. Construct 3 comprises an ER signal sequence, a vacuolar targeting domain and a pro-AEP domain. Constructs 2 and 3 are to be co-transformed. Filled triangles denote AEP cleavage sites; open triangles denote cleavage of the vacuolar targeting signal. FIG. 20B is a diagrammatic representation of alternative constructs for Pichia pastoris transformation. Constructs 4 and 5 are identical to Constructs 1 and 2 respectively (FIG. 20A) except for the addition of a kalata B1 N-terminal repeat (NTR) between the vacuolar targeting signal and the cyclotide domain.

FIG. 21 is a graphical representation showing expression of OaAEP1_{b} in Pichia pastoris when kalata B1 and AEP were expressed from the same transcriptional unit (SEQ ID NOs: 77 and 78). Samples were analysed by SDS-PAGE followed by Western blotting with anti-AEP1_{b} polyclonal rabbit serum. The negative control shows an unrelated protein expressed and extracted under the same conditions. T, total protein; L, total protein after lysis; S, soluble protein after lysis; C, concentrated soluble protein after lysis; +ve, positive control, rOaAEP1_{b} prior to activation.

FIG. 22 is a schematic representation of polypeptide ligation catalyzed by rOaAEP1_{b} between a first peptide (NaD1) having a C-terminal flanking sequence incorporating the rOaAEP1_{b} cleavage site and a 6xHis tag and a second peptide containing an N-terminus compatible with rOaAEP1_{b}. The leaving group on the first peptide is underlined.

### DETAILED DESCRIPTION

Throughout this specification, unless the context requires otherwise, the word “comprise”, or variations such as “comprises” or “comprising”, will be understood to imply the inclusion of a stated element or integer or method step or group of elements or integers or method steps but not the exclusion of any other element or integer or method step or group of elements or integers or method steps.

As used in the subject specification, the singular forms “a”, “an” and “the” include plural aspects unless the context clearly dictates otherwise. Thus, for example, reference to “a cyclic peptide” includes a single cyclic peptide, as well as two or more cyclic peptides; reference to “an AEP” includes a single AEP, as well as two or more AEPs; reference to “the disclosure” includes a single and multiple aspects taught by the disclosure; and so forth. Aspects taught and enabled herein are encompassed by the term “invention”. All such aspects are enabled within the width of the present invention.

The present specification teaches a method of producing a cyclic peptide and a peptide conjugate. The term “cyclic peptide” encompasses but is not limited to a “cyclotide”. A cyclic peptide is a peptide that is cyclic by virtue of backbone cyclization. It may be naturally cyclic or derived from a non-naturally cyclic linear polypeptide precursor. Hence, the polypeptide precursor from which the peptide is derived may be a natural substrate for cyclization or it may be a naturally linear peptide which is adapted for cyclization. The term “peptide” includes a polypeptide and a protein. For the avoidance of doubt, reference, for example, to a “cyclic peptide”, “polypeptide precursor”, “conjugate peptide” and the like is not to exclude a “cyclic polypeptide” or “cyclic protein”, a “precursor peptide” or “precursor protein” or a “conjugate polypeptide” or “conjugate protein”.

The method comprises the co-incubation either in a receptacle or in a cell of: (i) an AEP with cyclization activity; and (ii) a linear polypeptide precursor of the cyclic peptide. The AEP catalyzes the processing of the polypeptide precursor to facilitate excision and circularization of the cyclic peptide. If in a receptacle, the cyclic peptide is purified. If cyclization is catalyzed in a cell, the cyclic peptide is isolated from a vacuole or other compartment within the cell. The term “peptide conjugate” means two or more peptides ligated together wherein at least one peptide comprises a C-terminal AEP recognition sequence and another peptide comprises an N-terminal AEP recognition sequence.

The linear polypeptide precursor comprises a C-terminal AEP processing site. Generally, but not exclusively, the C-terminal processing site is an amino acid sequence defined as comprising P3 to P1 prior to the actual cleavage site and comprising P1′ to P3″ after the cleavage site towards the C-terminal end. In an embodiment, P3 to P1 and P1′ to P3′ have the amino acid sequence:

X₂X₃X₄X₅X₆X₇

wherein X is an amino acid residue and:

X₂ is optional or is any amino acid;

X₃ is optional or is any amino acid;

X₄ is Nor D;

X₅ is G or S;

X₆ is L or A or I; and

X₇ is optional or any amino acid.

In an embodiment, X₂ through X₇ comprise the amino acid sequence:

X₂X₃NGLX₇

wherein X₂ X₃ and X₇ are as defined above.

The N-terminal end of the linear polypeptide precursor may contain no specific AEP processing site or may contain a processing site defined by any one of P1″ through P3″ wherein P1″ to P3″ is defined by:

X₉X₁₀X₁₁

wherein X is an amino acid residue:

X₉ is optional and any amino acid or G, Q, K, V or L;

X₁₀ is optional or any amino acid or L, F or I or an hydrophobic amino acid residue;

X₁₁ is optional and any amino acid.

In an embodiment, X₉ through X₁₁ comprise the amino acid sequence:

GLX₁₁

wherein X₁₁ is defined as above.

In an embodiment, the AEP processing site comprises N- and C-terminal end sequences comprising the sequence:

G_{LX11} [Xₙ]X₁X₂NGLX₆

wherein X₁₁, X₂, X₃, and X₇ are as defined above and [Xₙ] is absent (n=0) or any amino acid residue in a sequence of from 1 to 2000 amino acids. Reference to “1 to 2000” includes 1 to 1000 and 1 to 500 such as but not limited to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999 and 2000.

In an embodiment, the C-terminal processing site comprises P4 to P1 and P1′ to P4′ wherein P1 to P4 and P1′ to P4′ comprise X₁X₂X₃X₄X₅X₆X₇X₈ wherein X₂ to X₇ are as defined above and X₇ is optional or any amino acid and X₈ is optional or any amino acid.

The present invention comprises various aspects in relation to the co-incubation of the AEP with cyclization activity and the linear polypeptide precursor which include:

(i) introducing into a prokaryotic or eukaryotic cell a genetic vector encoding an AEP which is expressed then the AEP isolated and used in an in vitro cyclization reaction to generate a cyclic peptide from a linear polypeptide precursor;

(ii) introducing into a prokaryotic or eukaryotic cell a genetic vector encoding a linear polypeptide precursor which is expressed and purified, optionally post-translationally modified to introduce a non-naturally occurring amino acid residue and then subject to cyclization in vitro using an AEP to form a cyclic peptide, this includes modifications in the cell such as the production of isotopically-labeled peptides; and

(iii) introducing into a prokaryotic or eukaryotic cell, single or multiple genetic vectors encoding an AEP and a polypeptide precursor which enables production of a cyclic peptide in a vacuole or other cellular compartment of the cell.

Aspect (ii) can be modified whereby the linear polypeptide precursor is synthetically produced or isolated from a particular source. A linear peptide conjugate can be generated in vitro or in in vivo. In the case of eukaryotic cells, the AEP and linear polypeptide precursor may be produced in different cells or different cellular compartments of the same cell, isolated then used in vitro. In the case of Aspect (ii), in a prokaryotic cell, in a non-limiting embodiment, the cyclic peptide is generated by co-expression with an AEP in the periplasmic space. The polypeptide precursor may be a natural substrate for cyclization or may normally be a linear peptide that is rendered cyclic. Making a cyclic form of a linear peptide can improve stability, efficacy and utility.

By “co-incubation” is meant co-incubation in vitro in a receptacle or reaction vessel as well as within a cell. In addition, the AEP also has ligase activity enabling the generation of peptide conjugates of at least two peptides wherein at least one peptide comprises a C-terminal AEP recognition sequence and at least one other peptide comprising an N-terminal AEP recognition sequence.

Hence, enabled herein is a method for producing a cyclic peptide, the method comprising introducing into the prokaryotic or eukaryotic cell genetic material which, when expressed, generates an AEP with cyclization ability, isolating the AEP and then incubating the AEP with a polypeptide precursor, optionally incorporating a post-translational modification to introduce a non-naturally occurring amino acid residue or cross-linkage bond or other modification for a time and under conditions sufficient to generate a cyclic peptide from the polypeptide precursor; or co-expressing genetic material encoding the AEP with cyclization ability and a linear polypeptide precursor in a prokaryotic or eukaryotic cell for a time and under conditions sufficient to generate a cyclic peptide in a vacuole or other cellular compartment of the cell. In addition, the AEP can catalyze a ligation reaction to conjugate two or more peptides wherein at least one peptide comprises a C-terminal AEP recognition sequence and another peptide comprises an N-terminal AEP recognition sequence. The cell can also be used to generate one or both of the AEP and/or polypeptide precursor for use in the generation of a cyclic peptide in vitro. In an embodiment, the cyclic peptide is produced by co-expression of an AEP with cyclization ability and a target polypeptide in the periplasmic space of a prokaryotic cell.

Further enabled herein is a method of generating a linear peptide conjugate the method comprising co-incubating two or more peptides wherein at least one peptide comprises a C-terminal AEP recognition sequence and at least one other peptide comprises an N-terminal AEP recognition sequence with an AEP for a time and under conditions sufficient for at least two peptides to ligate together to form a peptide conjugate.

As indicated above, reference to a “peptide” includes a polypeptide and a protein. No limitation in the size or type of proteinaceous molecule is intended by use of the terms “peptide”, “polypeptide” or “protein”.

A “vector” refers to a recombinant plasmid or virus that comprises a polynucleotide to be delivered into a host cell. The polynucleotide to be delivered comprises a coding sequence of AEP and/or the polypeptide precursor or multiple forms of the same or different peptides. The term includes vectors that function primarily for introduction of DNA or RNA into a cell and expression vectors that function for transcription and/or translation of the DNA or RNA. Also included are vectors that provide more than one of the above functions.

A vector in relation to a prokaryotic or eukaryotic cell includes a multi-gene expression vehicle. Such as a vehicle consists of a polynucleotide comprising two or more transcription unit segments, each segment encoding an AEP or linear polypeptide precursor, each segment being joined to the next in a linear sequence by a linker segment encoding a linker peptide, the transcription segments all being in the same reading frame operably linked to a single promoter. Multiple polypeptide repeats or multiple different polypeptides may also be generated. A vector also includes a viral expression vector which comprises a viral genome with a modified nucleotide sequence which encodes a protein and enable stable expression. Alternatively, multiple vectors are used each encoding either an AEP or linear polypeptide precursor.

A “transcription unit” is a nucleic acid segment capable of directing transcription of a polynucleotide or fragment thereof. Typically, a transcription unit comprises a promoter operably linked to the polynucleotide that is to be transcribed, and optionally regulatory sequences located either upstream or downstream of the initiation site or the termination site of the transcribed polynucleotide. Alternatively, as a multigene expression vehicle, a single promoter and terminator is used to produce more than one protein from a single transcription unit A transcription unit includes a unit encoding either an AEP or a polypeptide precursor, or both.

A eukaryotic cell includes a yeast, a filamentous fungus and a plant cell. A “yeast cell” includes a species of Pichia such as but not limited to Pichia pastoris as well as Saccharomyces or Kluyveromyces. Other eukaryotic cells include non-human mammalian cells and insect cells. A prokaryotic cell includes an E. coli or some other prokaryotic microorganism suitable for production of recombinant proteins.

A “host” cell encompasses a prokaryotic cell (e.g. E. coli) or eukaryotic cell (e.g. a yeast cell such as a species of Pichia).

The terms “nucleic acid”, “polynucleotide” and “nucleotide” sequences are used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from the lineage of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. The polynucleotide encodes an AEP or linear polypeptide precursor including a linear precursor of a protein to be cyclized or two linear peptides to be ligated or any selectable marker.

A “gene” refers to a polynucleotide containing at least one open reading frame that is capable of encoding an AEP or polypeptide precursor after being transcribed and translated.

As used herein, “expression” refers to the process by which a polynucleotide transcription unit is transcribed into mRNA and/or the process by which the transcribed mRNA (also referred to as “transcript”) is subsequently translated into an AEP or polypeptide precursor. The transcripts and the encoded polypeptides are collectedly referred to as a “gene product”.

In the context of a linear polypeptide precursor, a “linear” sequence is an order of amino acids in the polypeptide in an N- to C-terminal direction in which amino acid residues that neighbour each other in the sequence are contiguous in the primary structure of the polypeptide. The “precursor” means it is a substrate for the AEP to generate a cyclic peptide. A linear peptide conjugate is generated following ligation of at least two peptides wherein at least one peptide comprises a C-terminal AEP recognition amino acid sequence and at least one peptide comprises an N-terminal AEP recognition amino acid sequence.

A “pathogen” includes a plant or animal or human pathogen selected from a fungus, insect, bacterium, nematode, helminth, mollusc, virus and a protozoan organism.

Enabled herein is a method for producing a cyclic peptide the method comprising co-incubating an AEP with peptide cyclizing activity and a linear polypeptide precursor of the cyclic peptide for a time and under conditions sufficient to generate the cyclic peptide. The co-incubation may occur in a receptacle (in vitro) or in a cell such as the vacuole or other cellular compartment of a cell. If the co-incubation is in vitro, then the AEP or the linear polypeptide precursor is produced in a prokaryotic or eukaryotic cell. The linear polypeptide precursor may also be produced in a cell and isolated and optionally post-translationally modified or synthetically generated to incorporate a non-naturally occurring amino acid residue or a non-naturally occurring cross-linkage bond or to be isotopically labeled. If co-incubation occurs in a cell, this may occur in a vacuole or other compartment of a eukaryotic cell or in a periplasmic space of a prokaryotic cell.

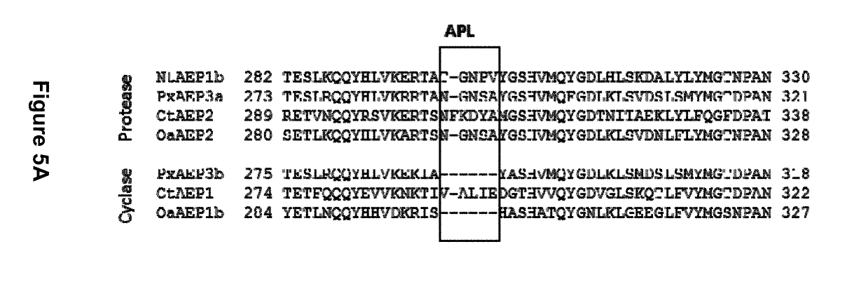

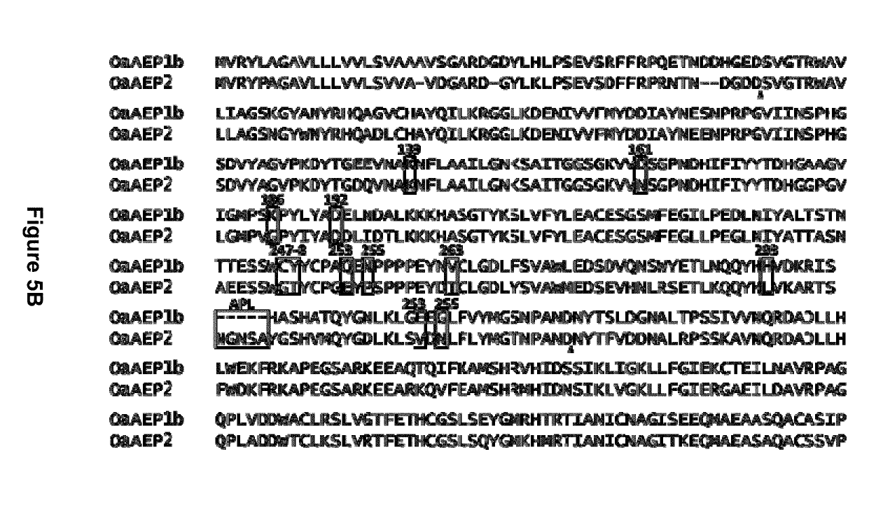

AEPs from cyclotide producing plants have been identified that, when expressed with the precursor gene for the cyclotide kalata B1 (oak1), and other peptides, are effective at backbone cyclization. By comparing the amino acid sequences of ligation competent AEPs with those favouring proteolysis, a differential loop region, termed the activity preference loop (APL), has been identified that contributes to the specificity. In ligase competent AEPs, the APL either has several residues missing or is replaced by hydrophobic stretch of amino acids (FIG. 5A).

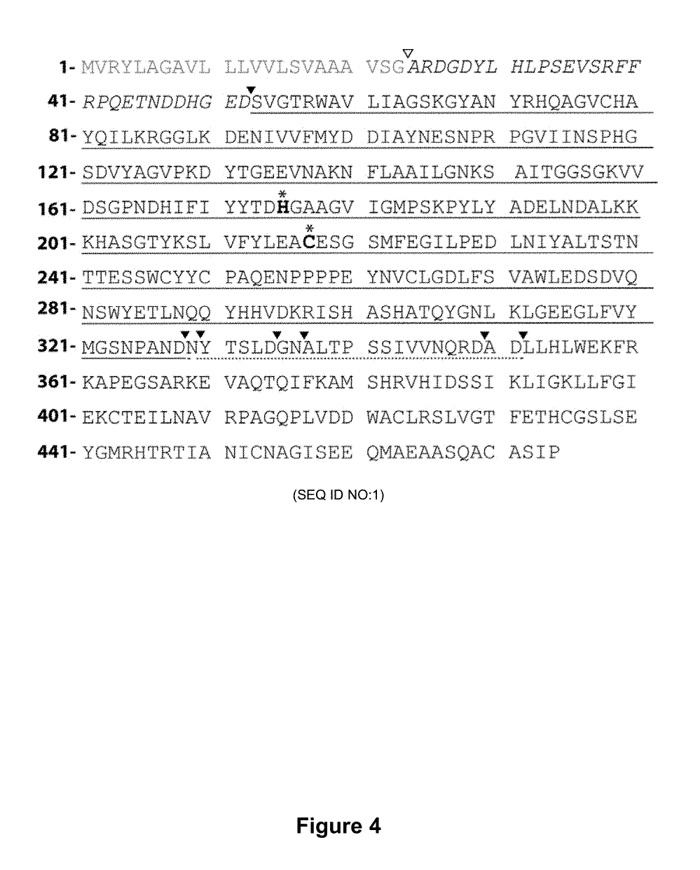

Additional residues linked to cyclase function are identified by machine learning (protein sequence space analysis) using a set of experimentally determined cyclase and non-cyclase sequences. The following residues are found to be highly predictive of cyclase function in the currently known cyclases and non-cyclases. All numbering is given relative to OaAEP1_{b} (FIG. 4; SEQ ID NO:1).

1. APL—The absence of residues in the region between 299-300 of OaAEP1 is predictive of a higher likelihood of cyclase activity.
2. Set 1—The presence of the following active site residues is also predictive of a higher likelihood of cyclase activity:
D161
C247
Y248
Q253
A255
V263
   3. Set 2—The presence of the following active site-proximal residues is also predictive of a higher likelihood of cyclase activity:
K186
D192
   4. Set 3—The presence of the following non-active site surface residues is also predictive of a higher likelihood of cyclase activity:
K139
H293
E314
G316

Overall it is highly predictive of cyclase activity if the sequence contains either:
The shortened APL
3 of the 6 Set 1 active site residues
Both of the Set 2 active-site-proximal residues
3 of the 4 Set 3 non-active-site residues

The most predictive are the APL and set 1. The more of these criteria that it hits, the more likely that it is to be a cyclase. Predictive residues for cyclase activity are shown in Table 2. Residue numbering is relative to OaAEP1_{b} (FIG. 4; SEQ ID NO:1). Residue properties that strongly predict cyclase activity are disorder propensity (DISORD), net static charge (CHRG), molecular weight of R group (RMW), and hydropathy index (HPATH).

An AEP having at least 25% or 5 or more of the 17 predictive residues set forth in Table 2 is considered likely to act preferentially as a cyclase. A requirement for at least 25% of the predictive residues to be present enables 100% of the known cyclases to be correctly identified while excluding known non-cyclases at least 80% of the time including at least 81, 82, 83, 84, 85, 86, 87, 88, 89, 90, 91, 92, 93 or 94% of the time. In an embodiment, the rule established herein enables exclusion of non-cyclases 94% of the time. One AEP excluded for being a non-cyclase is OaAEP2 (SEQ ID NO:3).

Accordingly, taught herein is a method for determining whether an AEP is likely to have cyclization activity, the method comprising determining the amino acid sequence of the AEP, aligning the sequence with a best fit to the amino acid sequence of OaAEP1_{b} (SEQ ID NO:1) and screening for the presence of 5 or more residues or absence of residues at 139K, 161D, 186K, 192D, 247C, 248Y, 253Q, 255A, 263V, 293H, Gap, Gap, Gap, Gap, Gap (between residues 299 and 300), 314E and 316G wherein gap means the absence of a residue wherein the presence of 5 or more of the listed residues or absence of residues is indicative of an AEP which is a cyclase.

In an embodiment, the from 5 to 17 residues or gaps screened at the listed sites include 5, 6, 7, 8, 10, 11, 12, 13, 14, 15, 16 or 17 residues or gaps. In an alternative representation, if the listed residues have at least 25% of the residues or gaps listed, then the AEP is deemed a cyclase. By “at least 25%” means 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

In yet a further alternative, the presence of 13 or more or 75% of the residues 139D, 161N, 186G, 192N, 247G, 248T, 253E, 255P, 263T, 293L, residues aligning between residues 299 and 300 of OaAEP1_{b}—N, G, N, Y and S, 314K and 316K is indicative of an AEP which is a non-cyclase. Reference to “13 or more” means 13, 14, 15, 16 and 17. Reference to “at least 75%” means 75, 76, 77, 78, 79, 80, 81, 82, 83, 84, 85, 86, 87, 88, 89, 90, 91, 92, 93, 94, 95, 96, 97, 98, 99 or 100%. Accordingly, enabled herein is a a method for determining whether an AEP is unlikely to have cyclization activity, the method comprising determining the amino acid sequence of the AEP, aligning the sequence with a best fit to the amino acid sequence of OaAEP1_{b} (SEQ ID NO:1) and screening for the presence of 13 or more of the residues 139D, 161N, 186G, 192N, 247G, 248T, 253E, 255P, 263T, 293L, residues aligning between residues 299 and 300 of OaAEP1_{b}—N, G, N, Y and S, 314K and 316K wherein the presence of 13 or more of the listed residues is indicative of an AEP which is not a cyclase.

The present invention extends to any AEP with peptide cyclization activity such as those defined above. Encompassed, herein, is any other AEP such as, but not limited to, OaAEP1_{b} (SEQ ID NO:1), OaAEP1 (SEQ ID NO:2) and OaAEP3 (SEQ ID NO:4) from Oldenlandia affinis. Other AEPs include an AEP having at least 80% amino acid similarity to SEQ ID NO:1 (OaAEP1_{b}), SEQ ID NO:2 (OaAEP1) or SEQ ID NO:4 (OaAEP3) after optimal alignment and which retains AEP and peptide cyclization activity and when the AEP comprises the presence of 5 or more of residues or absence of residues at 139K, 161D, 186K, 192D, 247C, 248Y, 253Q, 255A, 263V, 293H, Gap, Gap, Gap, Gap, Gap (between residues 299 and 300), 314E and 316G wherein Gap means the absence of a residue when optimally aligned to SEQ ID NO:1. The AEP may also have ligase activity to facilitate generation of peptide conjugates. OaAEP2 (SEQ ID NO:3) is an example of an AEP which is not a cyclase. It is a proviso that statements encompassing cyclase AEPs do not include OaAEP2 (SEQ ID NO:3).

In a prokaryotic cell, the first N-terminal residue in a construct is necessarily methionine. In the event that an N-terminal methionine precludes cyclization, alternative approaches are utilized. For example:

The endogenous methionine amino peptidase expressed by prokaryotic cells is harnessed to remove the initiating methionine in vivo, revealing an N-terminus appropriate for cyclization (Camarero et al. (2001) supra).

A recognition sequence for a protease that cleanly releases the additional residues (e.g. TEV protease, Factor Xa) is added N-terminal to the polypeptide precursor, exposing an appropriate N-terminus for cyclization following cleavage.

Reference to “at least 80%” includes 80, 81, 82, 83, 84, 85, 86, 87, 88, 89, 90, 91, 92, 93, 94, 95, 96, 97, 98, 99 and 100%.

The term “similarity” as used herein includes exact identity between compared sequences at the amino acid level. Where there is non-identity at the amino acid level, “similarity” includes amino acids that are nevertheless related to each other at the structural, functional, biochemical and/or conformational levels. In a particularly preferred embodiment, amino acid sequence comparisons are made at the level of identity rather than similarity.

Terms used to describe sequence relationships between two or more polypeptides include “reference sequence”, “comparison window”, “sequence similarity”, “sequence identity”, “percentage of sequence similarity”, “percentage of sequence identity”, “substantially similar” and “substantial identity”. A “reference sequence” includes from at least 10 amino acid residues (e.g. from 10 to 100 amino acids). A “comparison window” refers to a conceptual segment of typically 10 contiguous amino acid residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (BLASTP 2.2.32+, GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (1997) Nucl. Acids. Res. 25: 3389-3402). A detailed discussion of sequence analysis can be found in Unit 19.3 of Ausubel et al. (In: Current Protocols in Molecular Biology, John Wiley & Sons Inc. 1994-1998).

The terms “sequence identity” and “sequence similarity” as used herein refers to the extent that sequences are identical or functionally or structurally similar on an amino acid-by-amino acid basis over a window of comparison. Thus, a “percentage of sequence identity”, for example, is calculated by comparing two optimally aligned sequences over the window of comparison, determining the number of positions at which the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e. the window size), and multiplying the result by 100 to yield the percentage of sequence identity. For the purposes of the present invention, “sequence identity” will be understood to mean the “match percentage” calculated by the BLASTP 2.2.32+ computer program using standard defaults. Similar comments apply in relation to sequence similarity.

In an embodiment, taught herein is a method for producing a cyclic peptide the method comprising co-incubating an AEP with peptide cyclization activity having an amino acid sequence with at least 80% similarity to a sequence selected from the group consisting of SEQ ID NO:1, SEQ ID NO:2 and SEQ ID NO:4, after optimal alignment and wherein the presence of 5 or more of residues or absence of residues at 139K, 161D, 186K, 192D, 247C, 248Y, 253Q, 255A, 263V, 293H, Gap, Gap, Gap, Gap, Gap (between residues 299 and 300), 314E and 316G wherein Gap means the absence of a residue and a linear polypeptide precursor of the cyclic peptide for a time and under conditions sufficient to generate the cyclic peptide.

In an embodiment, enabled herein is a method for producing a cyclic peptide in vitro the method comprising introducing into a prokaryotic cell an expression vector encoding an AEP, enabling expression of the vector to produce a recombinant AEP, isolating the AEP from the cell and co-incubating in a reaction vessel the recombinant AEP with a polypeptide precursor for a time and under conditions sufficient to generate the cyclic peptide.

Taught herein is a method for producing a cyclic peptide in vitro the method comprising introducing into a prokaryotic or eukaryotic cell an expression vector encoding one or other of an AEP with peptide cyclization activity and a linear polypeptide precursor, enabling expression of the vector to produce a recombinant AEP and recombinant linear polypeptide precursor in the cell or component of the cell or a periplasmic space and isolating a cyclic peptide generated from the polypeptide precursor.

Enabled herein is a method for producing a cyclic peptide in vitro the method comprising introducing into a prokaryotic or eukaryotic cell an expression vector encoding an AEP with peptide cyclization activity, isolating the AEP and co-incubating in a reaction vessel the AEP with a polypeptide precursor for a time and under conditions sufficient to generate the cyclic peptide.

The polypeptide precursor may be recombinant or synthetically produced. The recombinant polypeptide may be post-translationally modified to introduce, or the synthetic form may incorporate, a non-naturally occurring amino acid.

Enabled herein is a method for producing a cyclic peptide in vivo the method comprising introduction into a prokaryotic or eukaryotic cell an expression vector encoding an AEP with peptide cyclization activity and a linear polypeptide precursor, enabling expression of the vector to produce the AEP and linear polypeptide precursor to produce a cyclic peptide. In an embodiment, this may occur in a periplasmic space or in a cellular compartment such as a vacuole.

In an embodiment, taught herein is a method for producing a cyclic peptide in vitro the method comprising introducing into a prokaryotic or eukaryotic cell an expression vector encoding one or other of an AEP with peptide cyclization activity or a linear polypeptide precursor, enabling expression of the vector to produce a recombinant AEP or recombinant linear polypeptide precursor and isolating the AEP or polypeptide from the cell and co-incubating in a reaction vessel the recombinant AEP with a polypeptide precursor or a post-translationally modified or synthetically modified form thereof for a time and under conditions sufficient to generate the cyclic peptide.

In an embodiment, the AEP comprises an amino acid sequence having at least 80% similarity to any one or more of SEQ ID NOs:1, 2 and/or 4 after optimal alignment.

As indicated above, reference to “at least 80%” means 80, 81, 82, 83, 84, 85, 86, 87, 88, 89, 90, 91, 92, 93, 94, 95, 96, 97, 98, 99 or 100%.

In another embodiment, a linear peptide is generated using the ligase activity of an AEP. In this embodiment, a first peptide comprising the C-terminal AEP recognition amino acid sequence is co-incubated with a second peptide with an N-terminal AEP recognition amino acid sequence and which may or may not have a tag and an AEP. The AEP catalyzes a ligation between the first and second peptides to generate a linear peptide conjugate. This may then subsequently be cyclized into a cyclic peptide or used as a linear peptide. This may occur in vitro or in vivo.

The polypeptide precursor may be a recombinant molecule generated by expression of nucleic acid encoding same in a cell or a combination of being produced by recombinant means followed by a post-translational modification (e.g. isotopically labeled) or produced by synthetic means. In relation to a post-translation modification or synthetic form, a non-naturally occurring amino acid may be introduced. A “cell” includes a prokaryotic (e.g. E. coli) or eukaryotic (e.g. a yeast) cell. The nucleic acid encoding the AEP and the polypeptide precursor may be present in two separate nucleic acid constructs or be part of a single construct such as a multi-gene expression vehicle. In either event, the nucleic acid is operably linked to a promoter which enables expression of the nucleic acid to produce the AEP and/or a linear form of the polypeptide precursor which is then processed into the cyclic peptide either in vitro or in vivo in a vacuole or other cellular compartment. In another embodiment, cells are maintained which are genetically modified to produce the AEP and these cells are then hosts for any given nucleic acid encoding a polypeptide precursor.

Taught herein is a method for producing a cyclic peptide in a cell, the method comprising introducing a genetic vector into the cell, the genetic vector comprising polynucleotide segments each encoding either an AEP with peptide cyclization activity or a polypeptide precursor, the polynucleotide segments separated by a polynucleotide linker segment wherein all polynucleotide segments are in the same reading frame operably linked to a single promoter and terminator wherein the eukaryotic cell is grown for a time and under conditions sufficient for a cyclic peptide to be generated which is then isolated from the vacuole or other cellular compartment.

Further taught herein is a method for producing a cyclic peptide in a cell, the method comprising introducing two genetic vectors in the cell, one encoding an AEP with peptide cyclization activity and the other encoding a polypeptide precursor, each genetic molecule comprising a promoter and terminator operably linked to polynucleotides encoding either the AEP or the polypeptide precursor wherein the cell is grown for a time and under conditions sufficient for a cyclic peptide to be generated which is then isolated from the vacuole or other cellular compartment.

In another embodiment, the vector encodes multiple repeats of the same polypeptide to be cyclized or multiple forms of different polypeptides to be cyclized.

In an embodiment, the AEP includes an AEP having at least 80% similarity to one or more of SEQ ID NOs:1, 2 and/or 4 after optimal alignment and wherein the presence of 5 or more of residues or absence of residues at 139K, 161D, 186K, 192D, 247C, 248Y, 253Q, 255A, 263V, 293H, Gap, Gap, Gap, Gap, Gap (between residues 299 and 300), 314E and 316G wherein Gap means the absence of a residue. Again, reference to “at least 80%” means 80, 81, 82, 83, 84, 85, 86, 87, 88, 89, 90, 91, 92, 93, 94, 95, 96, 97, 98, 99 or 100%. OaAEP2 (SEQ ID NO:3) is an example of a non-cyclase AEP. A eukaryotic cell includes a yeast cell such as a species of Pichia or a Saccharomyces sp. or Kluyveromyces sp.

Further taught herein is a method for generating a peptide conjugate comprising two or more peptides, the method comprising co-incubating at least two peptides with an AEP wherein at least one peptide comprises a C-terminal AEP recognition amino acid sequence and at least one other peptide comprises an N-terminal AEP recognition amino acid sequence. This may occur in vitro or in vivo.

Techniques and agents for introducing and selecting for the presence of a vector in cells are well-known. Genetic markers allowing for the selection of the vector cells are well-known, e.g. genes carrying resistance to an antibiotic such as kanamycin, tetracycline or ampicillin. The marker allows for selection of successfully transformed cells growing in the medium containing the appropriate antibiotic because they will carry the corresponding resistance gene. Eukaryotic cell selection of transformed cells is often accomplished through the inclusion of auxotrophic markers in the vector such as HIS4 or URA3 which encode enzymes involved in synthesis of essential amino acids or nucleotides. These vectors are then transformed into a yeast strain that is unable to synthesize specific amino acids or nucleotides that are required for growth, such as histidine for HIS4 and uracil for URA3. Cells that have been successfully transformed with the vector are selected by plating on dropout media lacking the specific amino acid or nucleotide as the untransformed cells are not able to synthesize the essential amino acid or nucleotide that is not present in the growth medium whereas cells carrying the vector with the auxotrophic marker survive as they are able to synthesize the missing amino acid or nucleotide. Other common auxotrophic markers are LEU2, LYS2, TRP1, HIS3, ARG4, ADE2.

Techniques for introducing an expression vector comprising a promoter operably linked to a polynucleotide into cell are varied and include transformation, electroporation, microinjection, particle bombardment or other techniques known to the art.

The choice of vector in which the nucleic acid encoding the AEP or polypeptide precursor is operatively linked depends directly, as is well known in the art, on the functional properties desired, e.g. replication, protein expression, and the host cell to be transformed, these being limitations inherent in the art of constructing recombinant nucleic acid molecules. For prokaryotic cells, the vector desirably includes a prokaryotic replicon, i.e. a DNA sequence having the ability to direct autonomous replication and maintenance of the recombinant DNA molecule extra-chromosomally when introduced into a prokaryotic cell. Such replicons are well known in the art. For eukaryotic cells, for example, the vector could either be maintained extra-chromosomally, in which case the vector sequence would generally comprise a eukaryotic replicon, or could be incorporated into the genomic DNA, in which case the vector would include sequences that would facilitate recombination of the vector into the host chromosome.

Those vectors that include a prokaryotic replicon also typically include convenient restriction sites for insertion of a recombinant DNA molecule of the present invention. Typical of such vector plasmids are pUC8, pUC9, pBR322, and pBR329 available from BioRad Laboratories (Richmond, Calif.) and pPL, pK and K223 available from Pharmacia (Piscataway, N.J.), and pBLUESCRIPT tm and pBS available from Stratagene (La Jolla, Calif.). A vector of the present invention may also be a Lambda phage vector as known in the art or a Lambda ZAP vector (available from Stratagene La Jolla, Calif.). Another vector includes, for example, pCMU (Nilsson et al. (1989) Cell 58:707-718). Other appropriate vectors may also be synthesized, according to known methods; for example, vectors pCMU/Kb and pCMUII used in various applications herein are modifications of pCMUIV (Nilsson et al. (1989) supra). The nucleic acid may be DNA or RNA.

Once introduced into a suitable host cell, expression of the nucleic acid can be determined using any assay known in the art. For example, the presence of a transcribed polynucleotide can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934). The polynucleotide encodes the AEP or polypeptide precursor or in the case of a eukaryotic system the polynucleotide may encode both.

Expression of the nucleic acid can also be determined by examining the protein product. A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), Western blot analysis, immunoprecipitation assays, immunofluorescent assays, and PAGE-SDS. In an embodiment, mass spectrometry is used for cyclic peptides (Saska et al. (2008) Journal of Chromatography B. 872:107-114).

In general, determining the protein level involves (a) providing a biological sample containing polypeptides; and (b) measuring the amount of any immunospecific binding that occurs between an antibody reactive to the AEP or polypeptide precursor, in which the amount of immunospecific binding indicates the level of expressed proteins. Antibodies that specifically recognize and bind to AEP or linear polypeptide precursor are required for immunoassays. These may be purchased from commercial vendors or generated and screened using methods well known in the art. See Harlow and Lane (1988) Antibodies: A Laboratory Manual, Cold Spring Harbor Laboratories. and Sambrook et al. (1989) Molecular Cloning, Second Edition, Cold Spring Harbor Laboratory, Plainview, N.Y. The sample of test proteins can be prepared by homogenizing the prokaryotic or eukaryotic transformants and optionally solubilizing the test protein using detergents, such as non-reducing detergents which include triton and digitonin. The binding reaction in which the AEP or polypeptide precursor is allowed to interact with the detecting antibodies may be performed in solution, or in cell pellets and/or isolated cells, for example, a solid support that has been immobilized with the test proteins. The formation of the complex can be detected by a number of techniques known in the art. For example, the antibodies may be supplied with a label and unreacted antibodies may be removed from the complex; the amount of remaining label thereby indicating the amount of complex formed. Results obtained using any such assay on a sample from a cell transformant is compared with those from a non-transformed source as a control. Other protein quantitation methods such as BCA and nanodrop methodologies may be employed.

The prokaryotic or eukaryotic host cells of this invention are grown under favorable conditions to effect expression of the polynucleotide. Examples of prokaryotic cells include E. coli, Salmonella sp, Pseudomonas sp and Bacillus sp. Examples of eukaryotic cells include yeast such as Pichia spp. (e.g. Pichia pastoris), Saccharomyces spp. or Kluyveromyces spp.

Accordingly, this invention provides genetically modified cells carrying one or two vectors encoding an AEP and/or a polypeptide precursor.

The present invention further contemplates a business model for producing cyclic peptides. In one embodiment, the business model comprises a prokaryotic cell encoding a heterologous AEP with cyclizing activity or a prokaryotic cell for use in introducing and expressing a vector encoding a desired linear polypeptide precursor. In either case, the polypeptide precursor produced by recombinant or synthetic means and the AEP are co-incubated in a reaction vessel for a time and under conditions sufficient for a cyclic peptide to be generated from the polypeptide precursor. In another embodiment, a prokaryotic or eukaryotic cell is selected for transformation with a vector encoding an AEP and a polypeptide precursor either in the same or separate constructs or the eukaryotic cell already comprises an AEP-encoding vector and is used as a recipient for a selected vector encoding a polypeptide precursor. The cell is then incubated for a time and under conditions sufficient for a cyclic peptide to form which can be isolated from the vacuole of the eukaryotic cell. The eukaryotic cell may be used to generate an AEP and/or polypeptide precursor which is used in vitro. In a further embodiment, the business model extends to the generation of linear peptide conjugates.

The cyclic peptides may have any of a range of useful properties including antipathogen, therapeutic or other pharmaceutically useful properties and/or insecticidal, molluscicidal or nematocidal activity. Examples of therapeutic activities include anticancer, protease inhibitory, antiviral or immunomodulatory activity and the treatment of pain. The cyclic peptide may also be a framework to incorporate a functionality. A normally linear polypeptide may also be subject to cyclization. This can improve stability, efficacy and utility. Alternatively, the polypeptide precursor is a natural substrate for cyclization.

As contemplated herein, a non-naturally occurring amino acid may be introduced into the polypeptide precursor. These include amino acids with a modified side chain.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during polypeptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids.

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids.

The polypeptide precursor may also be isotopically labeled by a cell or during in vitro synthesis.

Further enabled herein is a pharmaceutical formulation comprising the cyclic peptide or linear peptide conjugate or a pharmaceutically acceptable salt thereof. Such a formulation has applications in treating human and non-human animal subjects.

The term “pharmaceutically acceptable salts” refers to physiologically and pharmaceutically acceptable salts of the peptides of the invention: i.e., salts that retain the desired biological activity of the parent compound and do not impart undesired toxicological effects thereto.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

Emulsions are typically heterogenous systems of one liquid dispersed in another in the form of droplets usually exceeding 0.1 μm in diameter. Emulsions may contain additional components in addition to the dispersed phases, and the active drug which may be present as a solution in either the aqueous phase, oily phase or itself as a separate phase. Microemulsions are included as an embodiment of the present invention.

The pharmaceutical formulations and compositions of the present invention may also include surfactants. The use of surfactants in drug products, formulations and in emulsions is well known in the art.

In one embodiment, the present invention employs various penetration enhancers to effect the efficient delivery of cyclic peptides such as to treat onychomycosis of the nails. In addition to aiding the diffusion of non-lipophilic peptides across cell membranes, penetration enhancers also enhance the permeability of keratin. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable.

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates.

Optimum dosages may vary depending on the relative potency of individual cyclic or linear peptides, and can generally be estimated based on EC₅₀'s found to be effective in in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the peptide in bodily fluids or tissues. Following successful treatment, it may be desirable to have the patient undergo maintenance therapy to prevent the recurrence of the disease state, wherein the peptide is administered in maintenance doses, ranging from 0.01 μg to 100 g per kg of body weight, once or more daily, to once every 20 years.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

The cyclic peptide or linear peptide conjugate may also be formulated into an agronomically acceptable composition for topical application to plants or seeds. Agronomically acceptable carriers are used to formulate a peptide herein disclosed for the practice of the instant method. Determination of dosages suitable for systemic and surface administration is enabled herein and is within the ordinary level of skill in the art. With proper choice of carrier and suitable manufacturing practice, the compositions such as those formulated as solutions, may be administered to plant surfaces including above-ground parts and/or roots, or as a coating applied to the surfaces of seeds.

Agronomically useful compositions suitable for use in the system disclosed herein include compositions wherein the active ingredient(s) are contained in an effective amount to achieve the intended purpose. Determination of the effective amounts is well within the capability of those skilled in the art, especially in light of the disclosure provided herein.

In addition to the active ingredients, these compositions for use against plant pathogens may contain suitable agronomically acceptable carriers comprising excipients and auxiliaries which facilitate processing of the active compounds into preparations which can be used in the field, in greenhouses or in the laboratory setting.

Anti-pathogen formulations include aqueous solutions of the active compounds in water-soluble form. Additionally, suspensions of the cyclic peptides may be prepared as appropriate oily suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Further components can include viscosifiers, gels, wetting agents, ultraviolet protectants, among others.

Preparations for surface application can be obtained by combining the active peptides with solid excipient, optionally grinding a resulting mixture, and processing the mixture of granules, after adding suitable auxiliaries, if desired, to obtain powders for direct application or for dissolution prior to spraying on the plants to be protected. Suitable excipients are, in particular, fillers such as sugars, including lactose, sucrose, mannitol, or sorbitol; cellulose or starch preparations, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

### EXAMPLES

Aspects disclosed herein are further described by the following non-limiting Examples.

### Materials and Methods

### Peptide Substrates and Inhibitors

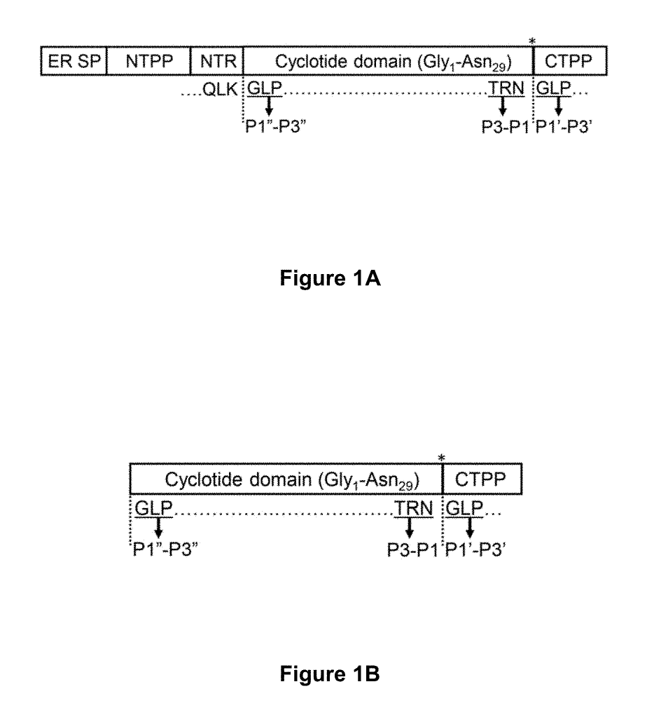

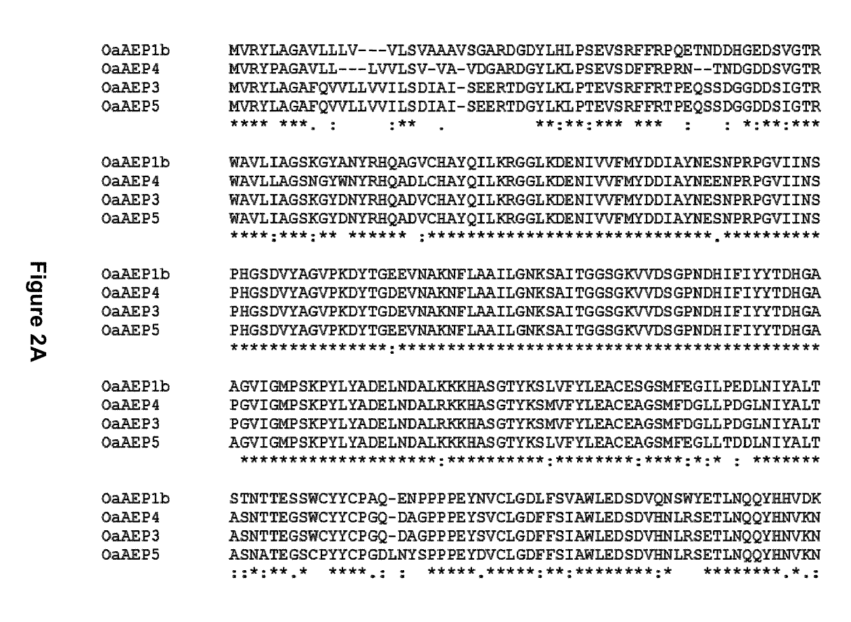

Two internally-quenched fluorescent (IQF) peptides (Abz-STRNGLPS-Y(3NO₂) (SEQ ID NO:21) and Abz-STRNGAPS-Y(3NO₂) (SEQ ID NO:25) where Abz is o-aminobenzoic acid and Y[3NO₂] is 3-nitrotyrosine) were synthesized by Genscript or GL Biochem at >90% purity and solubilized in 25% (v/v) acetonitrile:water. The fluorogenic peptide substrate Z-AAN-MCA (where Z is carboxybenzyl and MCA is 7-amido-4-methylcoumarin) was obtained from the Peptide Institute and solubilized in DMSO. The inhibitors Ac-YVAD-CHO and Ac-STRN-CHO (where Ac is acetyl and CHO is aldehyde) were synthesized by the Peptide Institute and Mimotopes respectively. The linear cyclotide precursor of kalata B1 was chemically synthesized and folded as described previously (Simonsen et al. (2004) FEBS Lett 577(3):399-402). This precursor was solubilized in ultrapure water and synthesized with a terminal free acid or amine FIG. 1 provides a representation of a linear cyclotide polypeptide precursor. Bac2A, EcAMP1 and R1 and its derivatives were synthesized with added AEP recognition residues by Genscript or GL Biochem at >85% purity with a terminal free acid or amine and solubilized in ultrapure water, except for one R1 derivative (LLVFAEFLPLFSKFGSRMHILKNGL; SEQ ID NO:89) which was solubilized in 25% DMSO. The ligation partner peptides (GLK-biotin; biotin-TRNGL; GLPVSGE, SEQ ID NO: 14; PLPVSGE, SEQ ID NO: 80) were synthesized by GL Biochem at >85% purity with a terminal free acid or amine and solublilized in ultrapure water.

### Cyclization Assay

Linear target peptides (280 μM, unless otherwise indicated) were incubated with rOaAEP1_{b}, rOaAEP3, rOaAEP4 or rOaAEP5 (total protein concentration as indicated in the description of figures) in activity buffer (50 mM sodium acetate, 50 mM NaCl, 1 mM ethylenediaminetetraacetic acid [EDTA], 0.5 μM Tris(2-carboxyethyl)phosphine hydrochloride [TCEP], pH 5). The reaction was allowed to proceed for up to 22 hours at room temperature and was analysed by matrix-assisted laser desorption/ionization mass spectrometry (MALDI MS), high performance liquid chromatography (HPLC) or nuclear magnetic resonance (NMR) as appropriate.

### Intermolecular Ligation Assays

Target peptides (140 μM) were incubated with a ligation partner peptide (700 μM) and a recombinant AEP (rOaAEP1_{b}, rOaAEP3, rOaAEP4 or rOaAEP5 at 19.7 μg mL⁻¹ total protein) in activity buffer (50 mM sodium acetate, 50 mM NaCl, 1 mM EDTA, 0.5 μM TCEP, pH 5). The reaction was allowed to proceed for up to 22 hours at room temperature and was analysed by MALDI MS or electrospray ionisation (ESI) mass spectrometry as appropriate.

### MS to Track AEP-Mediated Processing of Linear Peptides

Cyclization or inter-molecular ligation of linear target peptides was monitored by MALDI or ESI MS. In both cases, the reaction mixture (5-50 μL) was de-salted using C18 zip tips (Millipore) and eluted in 4 μL 50-75% v/v acetonitrile, 0.1% v/v trifluoroacetic acid (TFA).

For MALDI MS, a saturated MALDI matrix solution (α-cyano-4-hyroxycinnamic acid, CHCA, Bruker) prepared in 95% v/v acetonitrile, 0.1% v/v TFA was diluted 1:22 such that the final matrix solution comprised 90% v/v acetonitrile, 0.1% v/v TFA and 1 mM NH₄H₂PO₄. Eluted samples were mixed 1:4 with the MALDI matrix, spotted onto a MALDI plate and analyzed by an Ultraflex III TOF/TOF (Bruker) in positive reflector mode. Data were analyzed using the flexAnalysis program (Bruker).

For ESI MS, 96 μl of 75% v/v acetonitrile, 01% v/v TFA was added to the de-salted sample. The sample was then injected into a MicroTOF Q (Bruker) and data was collected in positive ionisation mode. The mass of ligated or cyclized product was determined by charge deconvolution using the Compass DataAnalysis program (Bruker).

### Assaying Protease Activity Against IQF and Fluorescent Peptides

To assay activity of recombinant AEPs (rOaAEP1_{b}, rOaAEP3, rOaAEP4 or rOaAEP5) against both internally-quenched and other fluorescent peptides, substrate and enzyme were diluted as appropriate in activity buffer (50 mM sodium acetate, 50 mM NaCl, 1 mM EDTA, 0.5 μM TCEP, pH 5). To assay activity of recombinant human legumain (rhuLEG; R&D systems) against the same substrates, the enzyme was first activated by incubation in 50 mM sodium acetate, 100 mM NaCl, pH 4 (4 μL buffer/1 μL enzyme) for 2 hours at 37° C. Substrates and activated rhuLEG were diluted in 50 mM acetate, 0.1% v/v triton X 100, 0.5 μM TCEP pH 5.5 or in 50 mM MES, 250 mM NaCl, pH 5 as required. Diluted enzyme and substrate were added to black, flat bottomed microtiter plates in a total assay volume of 100-200 μL. The change in fluorescence intensity over time was monitored on a SpectraMax M2 (Molecular Devices) using excitation/emission wavelengths of 320/420 nm (IQF peptides) or 360/460 nm (other fluorescent peptides).

### Inhibition Assays

To investigate the impact of inhibitors on enzyme activity against the wild type IQF peptide, Abz-STRNGLPS-Y(3NO₂), rOaAEP1_{b} (4.4 μg mL⁻¹ total protein) was incubated with Ac-YVAD-CHO (500 μM) or Ac-STRN-CHO (409 μM) for 40 minutes prior to addition to the substrate (11 μM). Enzyme activity against the wt IQF peptide was then assessed as described above.

### Antibodies

Polyclonal anti-AEP1_{b} rabbit serum was generated by immunizing a New Zealand White rabbit with a denatured, inactive form of O. affinis AEP1_{b} (residues D47-P474) that was produced recombinantly in E. coli. The rabbit received three doses, four weeks apart of 150 μg of antigen in 50% (v/v) phosphate-buffered saline (PBS) and Freund's incomplete adjuvant (Sigma). Serum was obtained two weeks after the final dose.

O. affinis Transcriptome

Total RNA was extracted from O. affinis root, leaf and seedling tissues using a phenol extraction method. Plant material was frozen in liquid nitrogen and ground to a fine powder, which was then resuspended in buffer (0.1 M Tris-HCl pH 8.0, 5 mM EDTA, 0.1 M NaCl, 0.5% SDS, 1% 2-mercaptoethanol), extracted twice with 1:1 phenol:chloroform and precipitated by addition of isopropanol. The pellets were dissolved in 0.5 ml water and RNA was precipitated overnight at 4° C. by addition of 4 M lithium chloride. The extracted RNA of each tissue was analysed by GeneWorks using the Illumina GAIIx platform. In total, 69.3 million 75 bp paired-end reads were generated. Reads were filtered with a phred confidence value of Q37 and assembled into contigs using Oases (Schulz et al. (2012) Bioinformatics 28: 1086-1096) with k-mer ranging from 41-67. The assemblies were merged using cd-hit-est (Li et al. (2006) Bioinformatics 22: 1658-1659), resulting in 270,000 contigs. Statistics on the depth of sequencing were made by aligning the reads of each tissue on the contigs using BWA (Li et al. (2009) Bioinformatics 25: 1754-1760). All the sequences, including one AEP, previously identified from an EST library of O. affinis were present among the contigs (Qin et al. (2010) BMC Genomics 11: 111). Homologues of this AEP sequence were searched using BLAST (Altschul et al. (1990) J Mol Biol 215: 403-410) in the contig library using a maximum E-value of 1e-20, resulting in the identification of 371 putative AEP transcripts. These sequences could then be clustered in 13 groups sharing at least 90% sequence identity using cd-hit (Li et al. (2006) supra). Coding sequences identified were OaAEP4-17 (SEQ ID NOs: 39 to 54).

### OaAEP Cloning

Full length AEP transcripts from the O. affinis transcriptome assembly were used to design a set of primers. A single degenerate forward primer (OaAEPdegen-F, 5′-ATG GTT CGA TAT CYC GCC GG-3′—SEQ ID NO:6) was manually designed to amplify all sequences due to the variability at a single nucleotide position within the 5′ region of each full length transcript at the start codon. Three reverse primers, designed with the aid of Primer3, successfully amplified AEP sequences (OaAEP1-R, 5′-TCA TGA ACT AAA TCC TCC ATG GAA AGA GC-3′—SEQ ID NO:7; OaAEP2-R, 5′-TTA TGC ACT GAA TCC TTT ATG GAG GG-3′—SEQ ID NO:8; OaAEP3-R 5′-TTA TGC ACT GAA TCC TCC ATC G-3′—SEQ ID NO:9).

To clone expressed OaAEPs, total RNA was extracted from O. affinis leaves and shoots using TRIzol (Life Technologies) and was reverse transcribed with SuperScript III reverse transcriptase (Life Technologies) according to the manufacturer's instructions. Target sequences were amplified from the resulting cDNA using Phusion High Fidelity Polymerase (New England BioLabs) and the primers described above under the recommended reaction conditions. Gel extracted PCR products were dA-tailed by incubation with Invitrogen Taq Polymerase (Life Technologies) and 0.5 μL 10 mM dA in the supplied buffer. The processed products were cloned into pCR8-TOPO (Life Technologies) and transformed into E. coli. Purified DNA from clones that were PCR positive for an AEP insert were sent for Sanger sequencing at the Australian Genome Research Facility. Coding sequences have been deposited in Genbank (accession codes: OaAEP1 (KR259377), OaAEP2 (KR259378), OaAEP3 (KR259379).

In a different approach, genomic DNA was extracted from O. affinis leaf tissue using a DNeasy Plant Mini Kit according to the manufacturer's instructions. PCR amplification from this DNA used primers specifically targeting the OaAEP1 nucleotide sequence. Gel extracted product was dA tailed as above, cloned into TOPO (Life Technologies) and transformed into E. coli. DNA from PCR-positive clones was sent for sequencing to the Australian Genome Research Facility. The AEP sequence identified using this method (OaAEP1_{b}) was subsequently expressed as a recombinant protein.

### Prediction of Cyclase Activity

AEPs are identified from cyclotide producing plants which, when expressed with the precursor gene for the cyclotide kalata B1 (oak1), and other peptides, effect backbone cyclization. By comparing the amino acid sequences of ligation competent AEPs with those favouring proteolysis, a differential loop region, termed the activity preference loop (APL), is identified that contributes to the specificity. In ligase competent AEPs, the APL either has several residues missing or is replaced by hydrophobic stretch of amino acids (FIG. 5A).

Additional residues linked to cyclase function are identified by machine learning (protein sequence space analysis) using a set of experimentally determined cyclase and non-cyclase sequences. The following residues are found to be highly predictive of cyclase function in the currently known cyclases and non-cyclases (FIG. 5B). All numbering is given relative to OaAEP1_{b} (FIG. 4; SEQ ID NO:1).

1. APL—The absence of residues in the region between 299-300 of OaAEP1 is predictive of a higher likelihood of cyclase activity.
2. Set 1—The presence of the following active site residues is also predictive of a higher likelihood of cyclase activity:
D161
C247
Y248
Q253
A255
V263
   3. Set 2—The presence of the following active site-proximal residues is also predictive of a higher likelihood of cyclase activity:
K186
D192
   4. Set 3—The presence of the following non-active site surface residues is also predictive of a higher likelihood of cyclase activity:
K139
H293
E314
G316

Overall it is highly predictive of cyclase activity if the sequence contains either:
The shortened APL
3 of the 6 Set 1 active site residues
Both of the Set 2 active-site-proximal residues
3 of the 4 Set 3 non-active-site residues

The most predictive are the APL and set 1. The more of these criteria that it hits, the more likely that it is to be a cyclase. Predictive residues for cyclase activity are shown in Table 2. Residue numbering is relative to OaAEP1_{b} (FIG. 4; SEQ ID NO:1). Residue properties that strongly predict cyclase activity are disorder propensity (DISORD), net static charge (CHRG), molecular weight of R group (RMW), and hydropathy index (HPATH).

An AEP having at least 25% (or 5 or more) of the 17 predictive residues set forth in Table 2 is considered likely to act preferentially as a cyclase. A requirement for at least 25% of the predictive residues to be present enables 100% of the known cyclases to be correctly identified while excluding known non-cyclases at least 80% including 94%.

Examples of AEPs predicted to be cyclases using this method include OaAEP4 (88%), OaAEP5 (70%), both sequences derived from transcriptome analysis, which have been tested and shown to be cyclases (e.g. Example 4). Other sequences predicted to be cyclases include AEPs from Cicer arietinum (SEQ ID NO:92), Medicago truncatula (SEQ ID NO:93), Hordeum vulgare (SEQ ID NO:94), Gossipium raimondii (SEQ ID NO:95 and Chenopodium quina (SEQ ID NO:96) (Example 10).

TABLE 2

| residue | | property | cyclase | non-cyclase |
|---|---|---|---|---|
| 139 | | CHRG | K | D |
| 161 | | CHRG | D | N |
| 186 | | CHRG | K | G |
| 192 | | CHRG | D | N |
| 247 | | RMW | C | G |
| 248 | | RMW | Y | T |
| 253 | | CHRG | Q | E |
| 255 | | DISORD | A | P |
| 263 | | HPATH | V | T |
| 293 | | HPATH | H | L |
| | {open oversize brace} | GAP | — | N |
| | | GAP | — | G |
| 299-300 | | GAP | — | N |
| | | GAP | — | Y |
| | | GAP | — | S |
| 314 | | CHRG | E | K |
| 316 | | RMW | G | K |

### Example 1

### Expression and Activation of Recombinant O. affinis AEPs (rOaAEPs) in E. coli

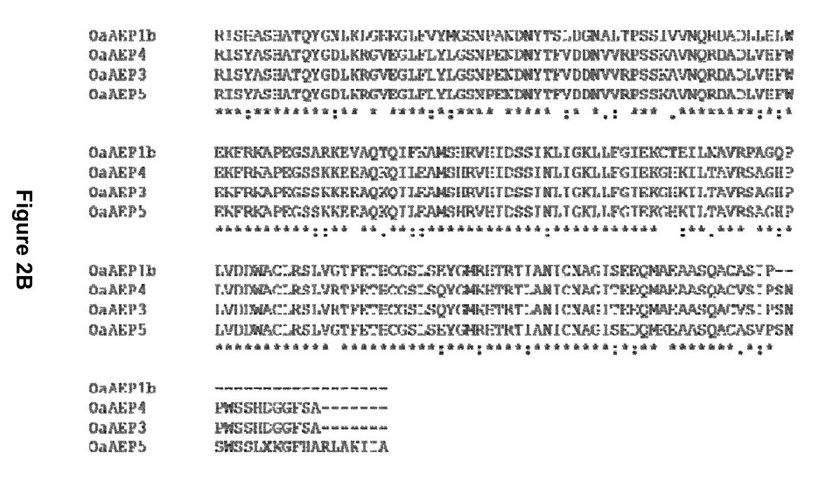

DNA encoding full-length O. affinis AEPs without the putative signalling domain (OaAEP1_{b} residues A₂₇-P₄₇₈, OaAEP3 residues R₂₈-A₄₉₁, OaAEP4 residues A₂₈-A₄₉₁ or OaAEP5 residues E₂₇-L₄₈₅) was inserted into the pHUE vector (Catanzariti et al. (2004) Protein Sci 13: 1331-1339) to give a 6xHis-ubiquitin-OaAEP fusion protein construct (SEQ ID NO:20 describes the rOaAEP1_{b} construct and the region containing OaAEP1_{b} is replaced with OaAEP3, OaAEP4 or OaAEP5 in the other constructs). Residue numbering is as determined by a multiple alignment generated using Clustal Omega (Sievers et al. (2011) supra) (FIG. 2). DNA was then introduced into T7 Shuffle E. coli cells (New England BioLabs). Transformed cells were grown at 30° C. in superbroth (3.5% tryptone [w/v], 2% yeast extract [w/v], 1% glucose [w/v], 90 mM NaCl, 5 mM NaOH) to mid-log phase; the temperature was then reduced to 16° C. and expression was induced with isopropyl ß-D-1-thiogalactopyranoside (IPTG; 0.4 mM; Bio Vectra) for approximately 20 hours. Cells were harvested by centrifugation and resuspended in non-denaturing lysis buffer (50 mM Tris-HCl, 150 mM NaCl, 0.1% triton X 100, 1 mM EDTA, pH 7). Lysis was promoted by a total of five freeze/thaw cycles and the addition of lysozyme (hen egg white; Roche; 0.4 mg mL⁻¹). DNase (bovine pancreas; Roche; 40 μg mL⁻¹) and MgCl₂ (0.4 M) were also added. Cellular debris was removed by centrifugation and the lysate was stored at −80° C. until required.

Lysate containing expressed recombinant AEPs was filtered through a 0.1 μM glass fibre filter (GE Healthcare) before being diluted 1:8 in buffer A (20 mM bis-Tris, 0.2 M NaCl, pH 7) and loaded onto two 5 mL HiTrap Q Sepharose high performance columns connected in series (GE Healthcare; 1.6-3.1 mL undiluted lysate mL⁻¹ resin). Bound proteins were eluted with a continuous salt gradient (0-30% buffer B [20 mM bis-Tris, 2 M NaCl, pH 7]; 15 column volumes [cv]) and AEP-positive fractions identified by Western blotting (anti-AEP1_{b} rabbit serum [1:2000]; peroxidase-conjugated anti-rabbit IgG [GE Healthcare; 1:5000]).

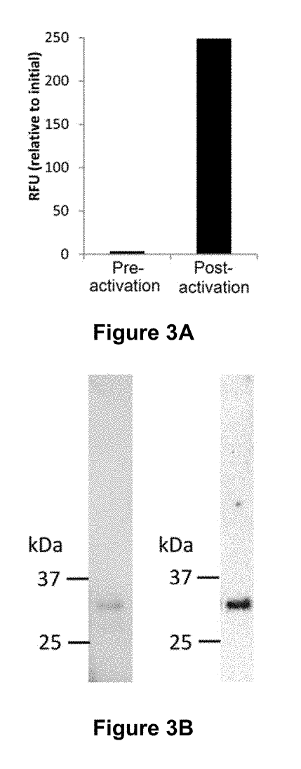

AEPs are usually produced as zymogens that are self-processed at low pH to their mature, active form (Hiraiwa et al. (1997) Plant J 12(4):819-829; Hiraiwa et al. (1999) FEBS Lett 447(2-3):213-216; Kuroyanagi et al. (2002) Plant Cell Physiol 43(2):143-151). To self-activate all AEPs, EDTA (1 mM) and TCEP (Sigma-Aldrich; 0.5 mM) were added, the pH was adjusted to 4.5 with glacial acetic acid and the protein pool was incubated for 5 hours at 37° C. FIG. 3A demonstrates that activity of rOaAEP1_{b} against an IQF peptide (Abz-STRNGLPS-Y(3NO₂)) representing the native C-terminal processing site in kB1 was dramatically increased following this activation step. Protein precipitation at pH 4.5 allowed removal of the bulk of the contaminating proteins by centrifugation. The remaining protein was filtered (0.22 μm; Millipore), diluted 1:8 in buffer A2 (50 mM acetate, pH 4) then captured on a 1 mL HiTrap SP Sepharose high performance column (GE Healthcare). Bound proteins were eluted with a salt gradient (0-100% buffer B2 [50 mM acetate, 1 M NaCl, pH 4]; 10 cv) and fractions with activity against an IQF peptide (Abz-STRNGLPS-Y(3NO₂)) were pooled and used in subsequent activity assays. FIG. 3B shows that after activation of rOaAEP1_{b}, a dominant band of ˜32 kDa was evident by reducing SDS-PAGE and Instant blue staining (Expedeon) and this was confirmed to be rOaAEP1_{b} by Western blotting. Experimentally determined self-processing sites of rOaAEP1_{b} are indicated in FIG. 4. The total concentration of protein in each preparation was estimated by BCA assay according to the manufacturer's instructions.

### Example 2

### In Vitro Cyclization of the Cyclotide Kalata B1 (kB1)

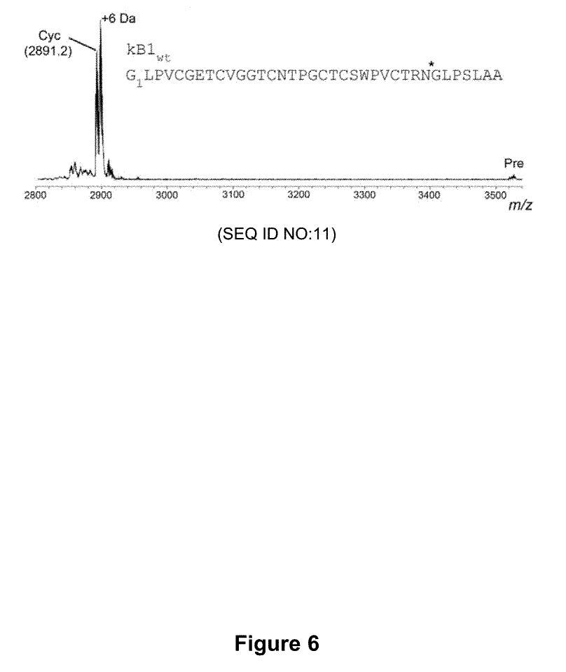

The ability of activated, mature rOaAEP1_{b} to cyclize a synthetic kB1 precursor carrying the native C-terminal pro-hepta-peptide (GLPSLAA) (FIG. 1B) was tested using the cyclization assay described in the Materials and Methods followed by MALDI MS. When incubated with the kB1 precursor the active enzyme produced a peptide of 2891.2 Da (monoisotopic, [M+H]⁺), consistent with the expected mass of mature, cyclic kB1 (FIG. 6). This product was confirmed to be identical to native kB1 by HPLC co-elution and 1D and 2D-NMR experiments.

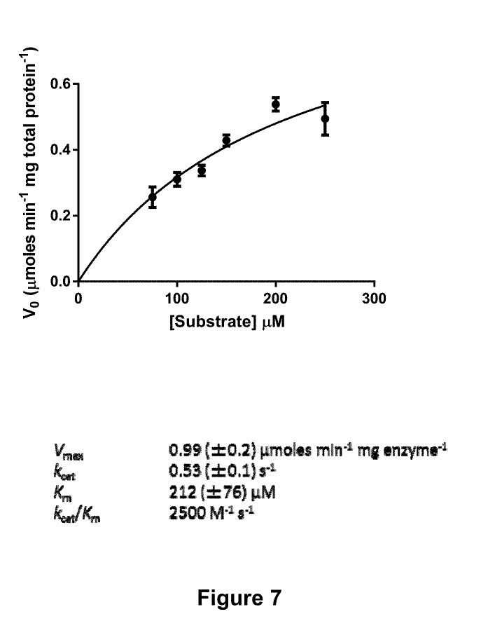

To determine the kinetics of rOaAEP1_{b} activity against the wt kB1 precursor (FIG. 1; SEQ ID NO: 11), the substrate was assayed at room temperature at a range of concentrations between 75 and 250 μM in a total volume of 20-160 μl of activity buffer. The total protein concentration of the enzyme preparation added to the kinetic assays was 19.7 μg ml⁻¹. The reaction was quenched after 5 min with 0.1% TFA and the volume adjusted to 800 μl. A volume of 700 μl was loaded onto a reversed-phase C18 analytical column (Agilent Eclipse C18, 5 μm, 4.6×150 mm) and peptides were separated by HPLC (19 min linear gradient of 12-60% acetonitrile, 0.1% TFA at 1 ml min⁻¹). The identity of eluted peaks was confirmed using MALDI MS. The area under the curve corresponding to the precursor peptide was quantitated by comparison to a standard curve and initial velocities were calculated by converting this to μmoles product formed. Kinetic parameters were estimated using the Michaelis-Menten equation and the curve-fitting program GraphPad Prism (GraphPad Software, San Diego). It was not possible to precisely determine the concentration of active enzyme due to impurities remaining in the preparation and the absence of an inhibitor appropriate for active site titration. However, a conservative turnover rate (k_{cat}) was estimated based on a mass of 32 kDa and the assumption that the total protein concentration reflected active enzyme. Kinetic parameters (±s.e.m.) for the processing of the wt kB1 precursor and rOaAEP1_{b} were 0.53 (±0.1) s⁻¹ for k_{cat}, 212 (±76) μM for Kₘ and 2,500 M⁻¹ s⁻¹ for k_{cat}/Kₘ as determined from a Michaelis-Menten plot (FIG. 7). Differences in purity and proportion of active enzyme in different preparations means these parameters are subject to batch to batch variation.

### Example 3

### In Vitro Cyclization of Non-Cyclotide Peptides R1, Bac2A and EcAMP1

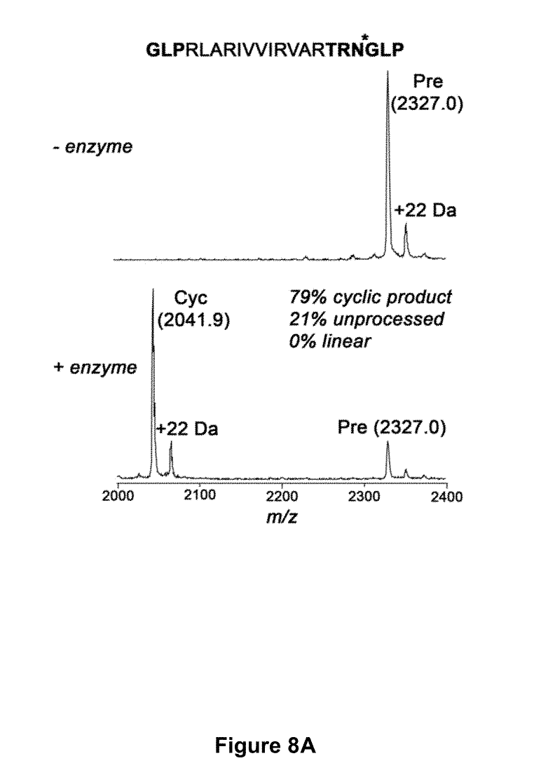

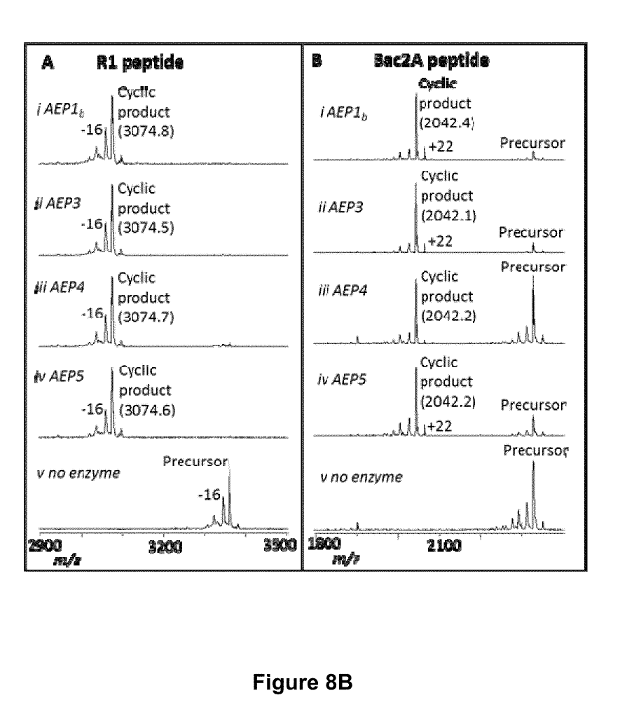

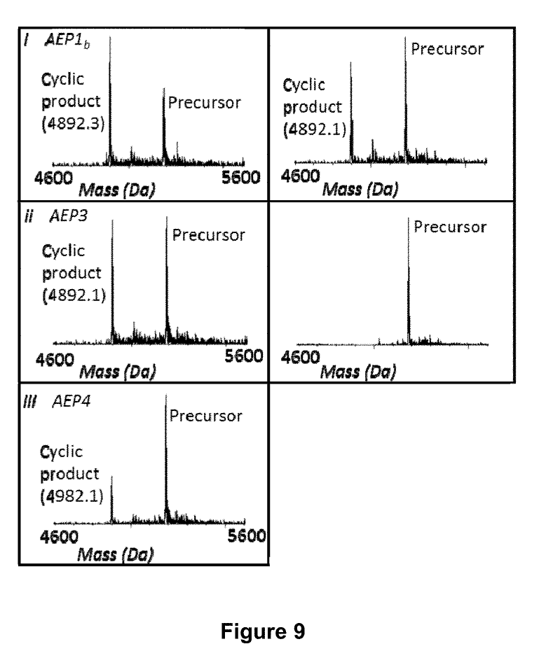

The ability of activated AEPs (rOaAEP1_{b}, rOaAEP3, rOaAEP4 and rOaAEP5) to cyclize peptide substrates structurally unrelated to cyclotides was tested in the cyclization assay described in the Materials and Methods. The anti-malarial peptide R1 (Harris et al. (2009) J Biol Chem 284(14):9361-9371; Harris et al. (2005) Infect Immun 73(10):6981-6989); Bac2A, a linear derivative of the bovine peptide bactenecin (Wu and Hancock (1999) Antimicrob Agents Ch 43:1274-1276); and the anti-fungal peptide EcAMP1 (Nolde et al. (2011) J Biol Chem 286(28):25145-25153) were produced with additional AEP recognition residues and used as the substrates. The appearance of a mass corresponding to cyclic product indicated that in each case the linear precursor peptides were efficiently cyclized following the addition of N- and C-terminal AEP recognition sequences (FIGS. 8 and 9).

### Example 4

### Sequence Requirements for In Vitro Cyclization

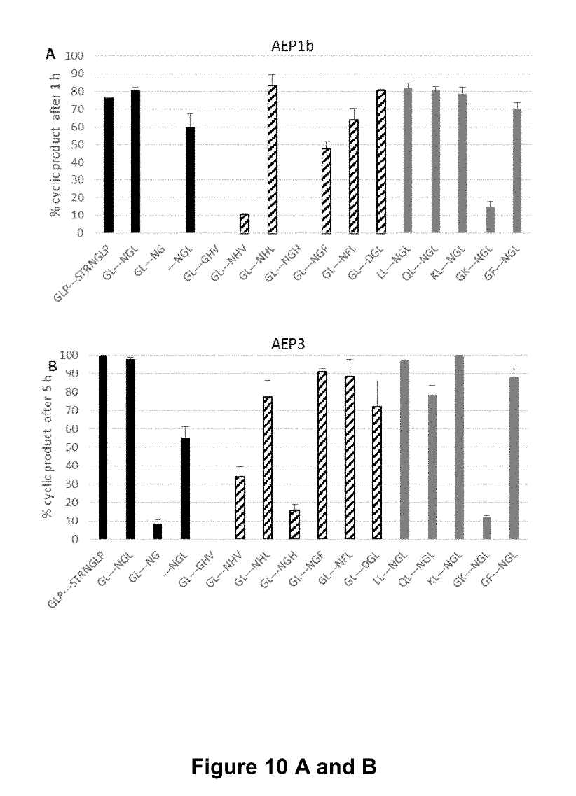

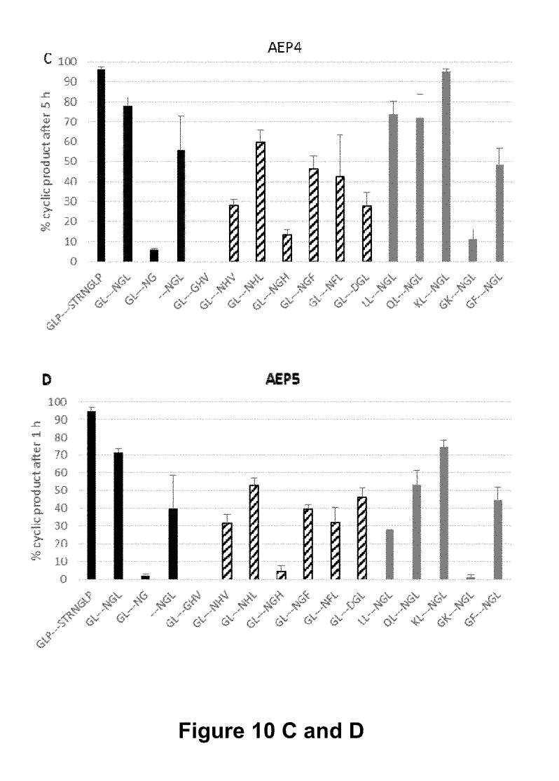

To investigate the sequence requirements for in vitro cyclization, R1 was used as a model peptide. The recognition residues added to this model peptide were sequentially trimmed to determine the minimal requirements for AEP-mediated cyclization. The N- and C-terminal recognition residues were also substituted with alternate amino acids to determine if particular classes of residues were preferred for cyclization by these recombinant AEPs. The ability of activated AEPs (rOaAEP1_{b}, rOaAEP3, rOaAEP4 and rOaAEP5) to cyclize the R1 peptide with varied flanking sequences was then tested in the cyclization assay described in the Materials and Methods (FIG. 10; Table 3).

Sequential trimming of the added recognition residues revealed that all four enzymes tested could cyclize the R1 peptide following the addition of only a C-terminal Asn-Gly-Leu motif (although some linear product was also produced from this precursor). After cleavage C-terminal to the Asn residue, only one residue (Asn) is left behind in the the cyclized peptide. However, more efficient cyclization was generally achieved with an N-terminal Gly-Leu motif as well as the C-terminal Asn-Gly-Leu motif. Subsequent mutations of the flanking residues were made within this format.

At the N-terminus, Leu, Gln and Lys were all accepted in place of the P1″ Gly, although in some cases this decreased the yield of cyclic product. Val was also accepted when presented as the first residue of the model peptide RE At the P2″ position, the positively charged Lys was poorly tolerated in place of Leu but cyclic product could still be produced. At the same position, the aromatic Phe was generally well accepted although again in some cases this decreased the yield of cyclic product at the timepoint tested. Any added amino acids at the N-terminus together with any added C-terminal amino acids up to and including the Asn are retained in the cyclic product. Therefore, for some target peptides where additional N-terminal residues impact function it may be acceptable to reduce the overall yield to minimize the introduction of non-native residues.

At the C-terminus, most substitutions resulted in a reduced yield of cyclic product under the conditions tested. At the P1′ position His and Phe could be accepted but the yield was generally reduced under the conditions tested. At the P2′ position Val, His and Phe could be accepted but this reduced yield in some cases. Other residues that could be accepted at this position are Ile, Ala, Met, Trp, Tyr. Residues C-terminal to the P1 residue are not incorporated into the final product. Therefore, there is little advantage to including sub-optimal residues within this region. All four enzymes tested were able to cyclize substrates presenting either an Asn or Asp in the P1′ position and preference was enzyme dependent. Since this residue is incorporated into the final peptide, choice of Asn or Asp at this position will likely be substrate dependent and this may influence which enzyme is selected for use.

No processing of either the native R1 peptide or a modified R1 carrying the N-terminal Gly-Leu motif but only an Asn at the C-terminus was observed by rOaAEP1_{b}. The cyclic nature of the R1 derivatives presented in Table 3 processed by rOaAEP1_{b} was confirmed by digestion with endoGlu-C(New England Biolabs; as per manufacturer's instructions). This secondary digestion produced a single linear product (as opposed to two linear peptides) consistent with linearization of a backbone cyclized peptide.

TABLE 3
The relative percentage of cyclic and linear
R1 peptide derivatives following rOaAEP
1_{b}-mediated processing.^{a,b,c}

| | Cyclic | Linear | Linear |
|---|---|---|---|
| | product | precursor | product |
| Peptide Sequence | (%) | (%) | (%) |
| GLPVFAEFLPLFSKF | 78.8 | 21.2 | — |
| GSRMHILKSTRNGLP | (±6.9) | (±6.9) | |
| (SEQ ID NO: 26) | | | |
| GLVFAEFLPLFSK | 92.9 | 7.1 | — |
| FGSRMHILKNGL | (±2.4) | (±2.4) | |
| (SEQ ID NO: 27) | | | |
| GLVFAEFLPLFSK | — | 100 | — |
| FGSRMHILKNG | | | |
| (SEQ ID NO: 28) | | | |
| VFAEFLPLFSKF | 49.6 | 27.7 | 27.7 |
| GSRMHILKNGL | (±14.1) | (±7.7) | (±7.3) |
| (SEQ ID NO: 29) | | | |
| QLVFAEFLPLFSK | 93.1 | 6.9 | — |
| FGSRMHILKNGL | (±2.2) | (±2.2) | |
| (SEQ ID NO: 30) | | | |
| KLVFAEFLPLFSK | 82.2 | 17.8 | — |
| FGSRMHILKNGL | (±3.8) | (3.8) | |
| (SEQ ID NO: 31) | | | |

^{a}The peak area of a given processing variant of R1 is displayed as a percentage of the total peak area attributable to that peptide.
^{b}The average of three experiments is reported ± standard error of the mean.
^{c}The enzyme concentration used was 12 μg mL⁻¹ total protein with an incubation time of 22 hours.

### Example 5

### Polypeptide Ligation

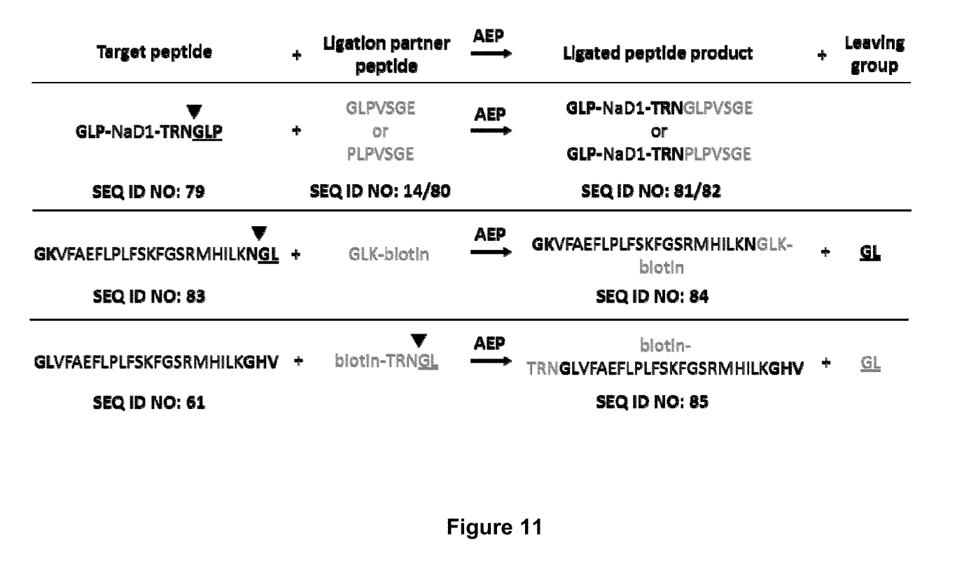

To investigate the ability of recombinantly produced AEPs to perform inter-molecular ligation (as well as the intra-molecular ligation required to produce backbone-cyclized peptides), target peptides were incubated with ligation partner peptides as well as active enzyme (FIG. 11). The appearance of new linear peptides were tracked. Ligation of labeled peptides to a target polypeptide provides a generic, targeted protein labeling strategy for a variety of moieties (e.g. fluorescent labels, biotin, affinity tags, epitope tags, solubility tags) that is limited only by the ability of synthetic peptide chemistry or other methods to produce the appropriate ligation partner. This approach can also enable ligation of multiple domains that could be challenging to produce as a single recombinant protein.

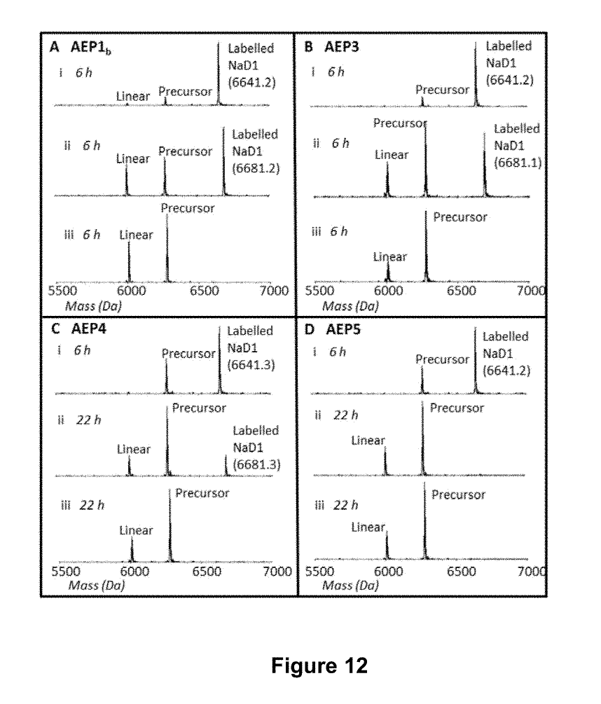

AEP recognition residues were added to the N- and C-termini of the plant defensin NaD1 (Lay et al. (2003) Plant Physiol 131:1283-1293) to produce a modified defensin (GLP-NaD1-TRNGLP; SEQ ID NO:79). This was recombinantly expressed in Pichia pastoris and purified using a similar method to that described in Lay et al. (2012) J Biol Chem 287:19961-19972. When AEP-mediated processing of the modified NaD1 (140 μM) was tested using the cyclization assay described in the Materials and Methods section, only a linear product was evident by ESI-MS and there was no evidence of backbone-cyclization (FIG. 12). Presumably the disulphide bonded structure of NaD1 is sub-optimal for cyclization. However, when the modified NaD1 was incubated with a ligation partner peptide (GLPVSGE, SEQ ID NO:14 or PLPVSGE, SEQ ID NO:80) and active, recombinant AEPs (rOaAEP1_{b}, rOaAEP3, rOaAEP4 and rOaAEP5) using the ligation assay described in the Materials and Methods, new, linear, peptides were detected using ESI-MS (FIG. 12).

The inter-molecular ligase activity of recombinant AEPs was further explored using using other peptide combinations. A modified R1 peptide (GKVFAEFLPLFSKFGSRMHILKNGL; SEQ ID NO:83) that was a poor substrate for backbone cyclization was used as a target peptide for C-terminal labelling. The R1 derivative was incubated with a biotinylated ligation partner (GLK-biotin; SEQ ID NO:102) and recombinant AEPs. MALDI-MS showed that AEP-mediated processing created a new linear peptide that incorporated a C-terminal biotin (FIG. 13).

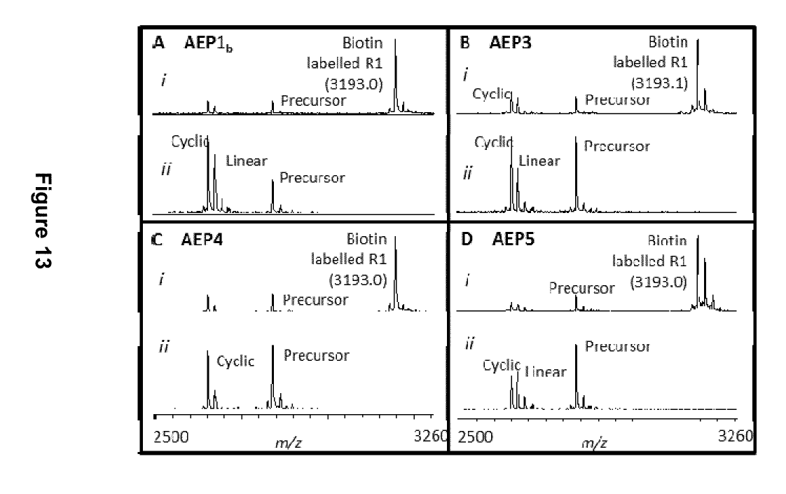

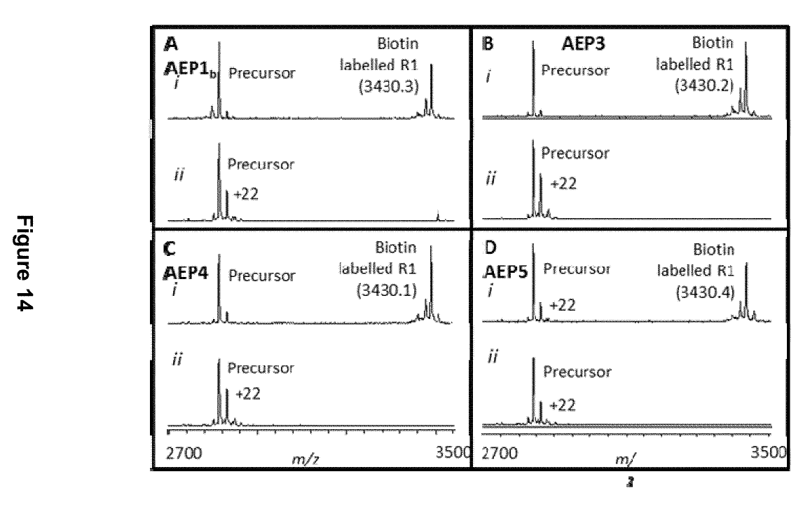

A modified R1 peptide, (GLVFAEFLPLFSKFGSRMHILKGHV; SEQ ID NO:61) that was not itself an AEP substrate since it does not contain the required Asx residue was used as a target peptide for N-terminal labelling. The R1 derivative was incubated with a biotinylated ligation partner peptide (biotin-TRNGL; SEQ ID NO:104) and recombinant AEPs. MALDI-MS showed that AEP-mediated processing created a new linear peptide that incorporated an N-terminal biotin (FIG. 14).

### Example 6

### Identification of Cyclizing AEPs by Substrate Specificity

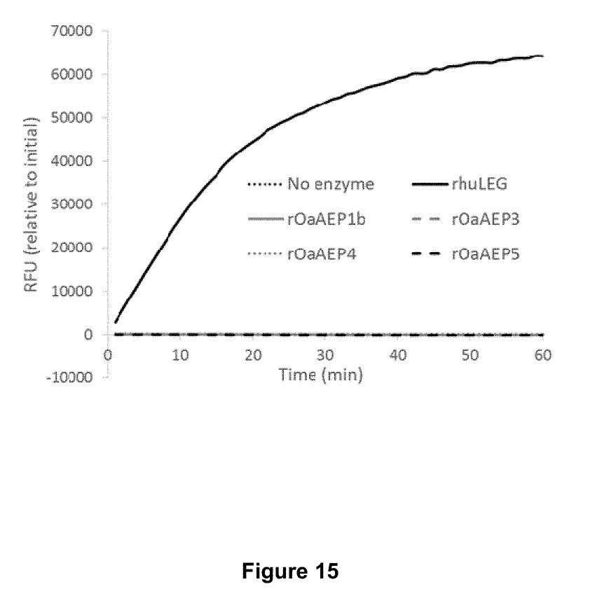

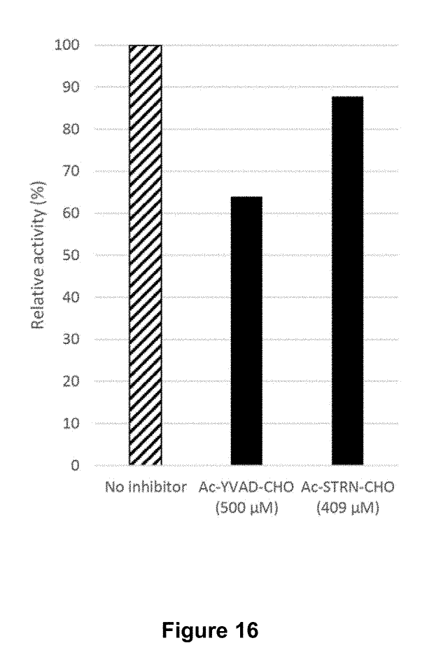

AEP activity has traditionally been tracked by monitoring cleavage of the fluorescent substrate Z-AAN-MCA (where Z is carboxybenzyl; MCA is 7-amido-4-methylcoumarin) [Saska et al. (2007) supra; Rotari et al. (2001) Biol. Chem. 382:953-959]. Cleavage C-terminal to the Asn liberates the fluorophore which then fluoresces to report substrate cleavage. However, neither butelase-1 (Nguyen et al. (2014) supra) nor rOaAEP1_{b}, rOaAEP3, rOaAEP4 or rOaAEP5 (FIG. 15) had detectable activity against this substrate. Furthermore, two AEP active site inhibitors had limited efficacy against rOaAEP1_{b} at high concentrations (FIG. 16). They are Ac-YVAD-CHO, which is routinely used to identify AEP activity (Hatsugai et al. (2004) Science 305(5685): 855-858) and Ac-STRN-CHO, which represents the P1-P4 residues of the C-terminal kB1 cleavage site. These traditional routes of identifying AEP activity will therefore likely be ineffective for identification of AEPs with cyclizing ability.

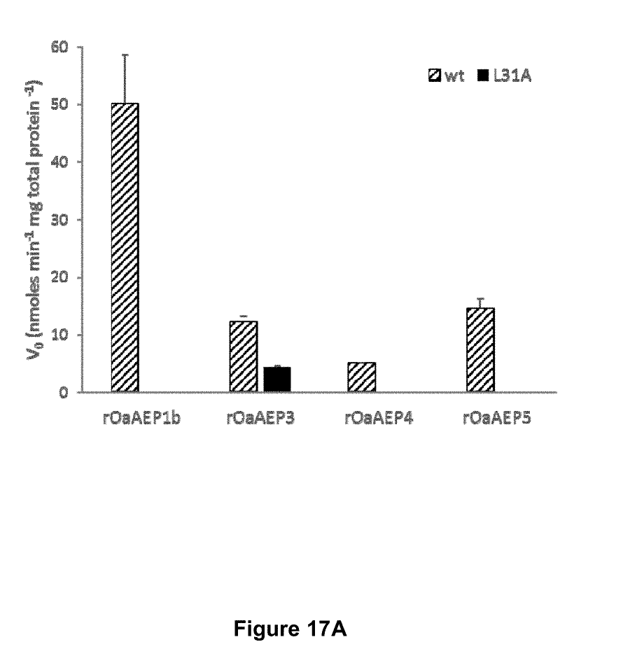

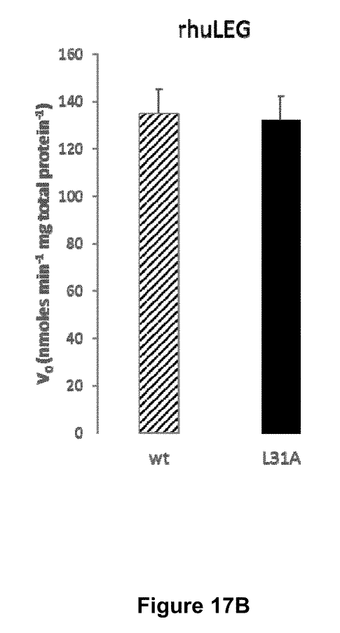

IQF peptides that incorporate the P1-P4 as well as the P1′-P4′ residues are, however, effectively targeted by recombinant O. affinis AEPs. These peptides contain a fluorescence donor/quencher pair, with fluorescence observed upon the spatial separation of this pair following enzymatic cleavage. Activity against such IQF reporter peptides without corresponding activity against the generic substrate (Z-AAN-MCA) may allow rapid identification of members of the AEP family likely to have cyclizing ability. In the IQF peptide format, rOaAEP1_{b} displayed a preference for a bulky hydrophobic residue such as Leu at the P2′ position that was not shared by human legumain (rhuLEG), an AEP that preferentially functions as a hydrolase (FIGS. 17A and 17B). Such P2′ specificity could also be used to predict cyclization ability and or to select AEPs with different sequence requirements in the substrate to be cyclized

### Example 7

### In Vitro Cyclization of Bacterially-Expressed Polypeptides

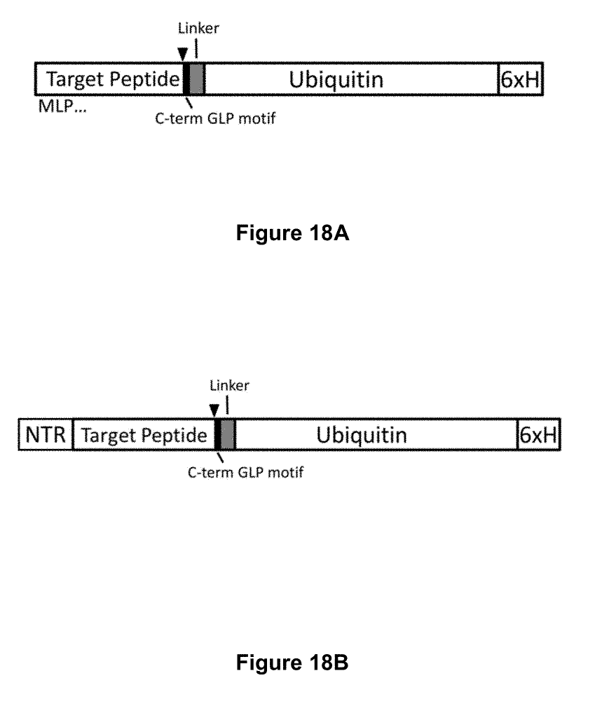

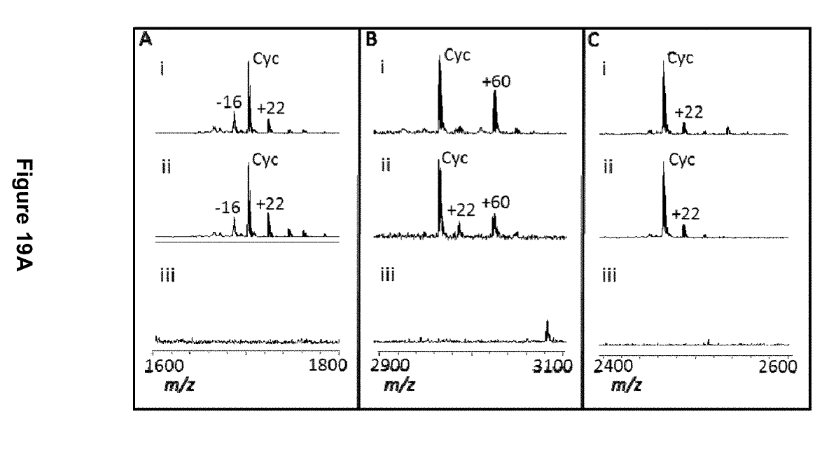

DNA encoding a target peptide for cyclisation, a short linker (Glu-Phe-Glu-Leu or Gly-Gly-Gly-Gly-Ser-Glu-Phe-Glu-Leu) and a C-terminal ubiquitin-6xHis was inserted into either the pHUE vector (Catanzariti et al. (2004) supra) (XbaI/BamHI) or the pET23a(+) vector (Invitrogen; NdeI/XhoI) to give a target peptide-linker-ubiquitin-6xHis fusion protein construct (FIG. 18 A). The linker coding region contains restriction sites for easy substitution of the target peptide domain with other target sequences. In the case of pHUE, the DNA sequence inserted included nucleotides prior to the initiating Met codon to ensure the original vector sequence was reconstituted. If not naturally present in the target peptide, appropriate N- and C-terminal AEP recognition sequences were introduced. Since the first residue of all target peptides was necessarily Met, the N-terminal recognition sequence added was Met-Leu. The C-terminal recognition sequence was Asn-Gly-Leu-Pro. Optionally, the target peptide is preceeded by an initiating Met followed by the kalata B1 N-terminal repeat (NTR) (FIG. 18 B) or other cleavable domain.

The target peptides produced as fusion proteins with ubiquitin were the cyclotide kB1 (SEQ ID NO:74), the modified sunflower trypsin inhibitor SFTI-1 I10R (Quimbar et al. (2013) J Biol Chem 288(19):13885-13896) (SEQ ID NO:72) and the conotoxin Vc1.1 (Clark et al. (2010) supra) (SEQ ID NO:76). The constructs were introduced into T7 Shuffle E. coli cells (New England BioLabs) and grown at 30° C. in 2YT (16% [w/v] tryptone, 10% [w/v] yeast, 5% [w/v] sodium chloride) to mid-log phase. The temperature was then reduced to 16° C. and expression was induced with IPTG (0.4 mM; Bio Vectra) for approximately 20 hours. Cells were harvested by centrifugation and resuspended in non-denaturing lysis buffer (50 mM sodium phosphate pH 8.0, 300 mM NaCl, 1-10 mM imidazole). Lysis was promoted by up to five freeze/thaw cycles and the addition of lysozyme (hen egg white; Roche; 0.4-1 mg mL⁻¹). DNase (bovine pancreas; Roche; 5 μg mL⁻¹) and MgCl₂ (5 mM) were also added. Cellular debris was removed by centrifugation. The lysate was then filtered through a 0.1 μM glass fibre filter (GE Healthcare) and passed over a Ni-NTA resin (QIAgen) to capture 6xHis tag protein. Bound protein was eluted with elution buffer (50 mM sodium phosphate pH 8.0, 300 mM NaCl, 250 mM imidazole) and the total protein concentration was estimated by BCA assay according to the manufacturer's instructions. Fusion proteins were then used in enzyme assays. Optionally, the eluted protein is first buffer exchanged into water or appropriate buffer before AEP processing is assayed. Optionally, the eluted protein is first further purified by diluting 1:10 in 20 mM Tris-HCl, pH8 and passing over as second resin (Q sepharose high performance anion exchanger; GE Healthcare). Bound protein is recovered by a continuous salt gradient (0-100% 20 mM Tris-HCl, 1M NaCl pH 8), optionally buffer exchanged into ultrapure water or appropriate buffer and concentrated.

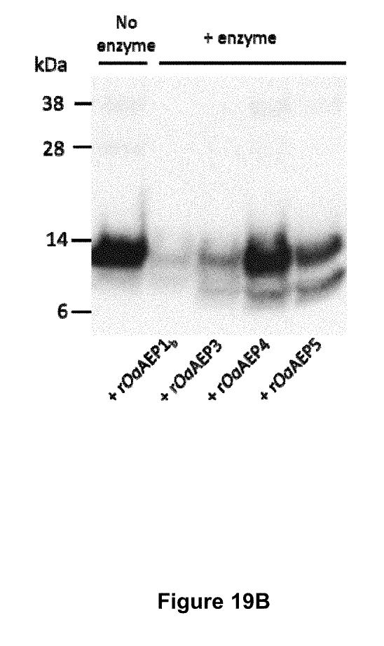

The ability of AEPs (rOaAEP1_{b}, rOaAEP3, rOaAEP4, rOaAEP5) to release the ubiquitin tag and cyclise target peptides was investigated using the cyclisation assay described in the Materials and Methods followed by MALDI MS (FIG. 19 A, Table 4). Estimated substrate and enzyme concentrations are as indicated in the description of the figures. When required, 3.3% v/v glacial acetic acid was also added to the reaction mix to ensure the assay was carried out at acidic pH. When incubated with the fusion proteins, all recombinant AEPs tested released ubiquitin and produced cyclized kB1 (SEQ ID NO: 73), SFTI-1 I10R (SEQ ID NO: 71) and Vc1.1 (SEQ ID NO: 75) in a single step. To estimate the proportion of fusion protein being enzymatically processed, loss of the precursor protein over time was tracked using SDS-PAGE followed by Western blotting (anti-6xHis monoclonal mouse antibody [Genscript; 0.5 μg mL⁻¹]; peroxidase-conjugated anti-mouse IgG [Thermo Scientific; 1:10000] (FIG. 19 B). This demonstrated that for rOaAEP1_{b}, rOaAEP3 and rOaAEP5 the bulk of the precursor was being enzymatically processed. In this experiment a smaller proportion of the precursor protein was processed by rOaAEP4.

Optionally, to separate released ubiquitin and unprocessed fusion protein from cyclized product, the mixture is then diluted 1:5 in non-denaturing lysis buffer and again passed over a Ni-NTA resin (QIAgen). The processed, cyclic, product no longer contains a 6xHis tag and is therefore present in the unbound fraction. This product is then dialysed into ultrapure water, concentrated and analysed by MALDI MS, HPLC or NMR to confirm its cyclic structure.

TABLE 4
The expected and observed monoisotopic masses of cyclic products following
AEP-mediated processing of target peptides fused to ubiquitin.^{a,b,c}

| | Monoisotopic cyclic mass (Da; [M + H]⁺) | | | | |
|---|---|---|---|---|---|
| Target | | Observed | Observed | Observed | Observed |
| peptide | Expected | (rOaAEP1_{b}) | (rOaAEP3) | (rOaAEP4) | (rOaAEP5) |
| SFTI1-I10R- | 1800.9 (ox) | | | | |
| ubiquitin | 1802.9 (red) | 1803.0 (red) | 1802.7 (red) | 1803.0 (red) | 1802.6 (red) |
| kB1- | 2965.2 (ox) | | | | |
| ubiquitin | 2971.2 (red) | 2965.6 (ox) | 2966.6 (ox) | 2965.2 (ox) | 2966.5 (ox) |
| Vc1.1- | 2460.9 (ox) | | | | |
| ubiquitin | 2464.9 (red) | 2464.7 (red) | 2465.3 (red) | 2464.7 (red) | 2465.3 (red) |

^{a}Ox, oxidized; red, reduced
^{b}Substrate concentrations: SFTI1-I10R-ubiquitin (1 mg mL⁻¹ total protein); kB1-ubiquitin (0.9 mg mL⁻¹ total protein); Vc1.1-ubiquitin (0.24 mg mL⁻¹ total protein)
^{c}Enzyme concentrations: rOaAEP1_{b} and rOaAEP5 (19.7-98.5 μg mL⁻¹ total protein); rOaAEP3 (19.7-21.9 μg mL⁻¹ total protein) and rOaAEP4 (19.7-30 μg mL⁻¹ total protein)

### Example 8

### In Vivo Cyclization of Yeast-Expressed Polypeptides

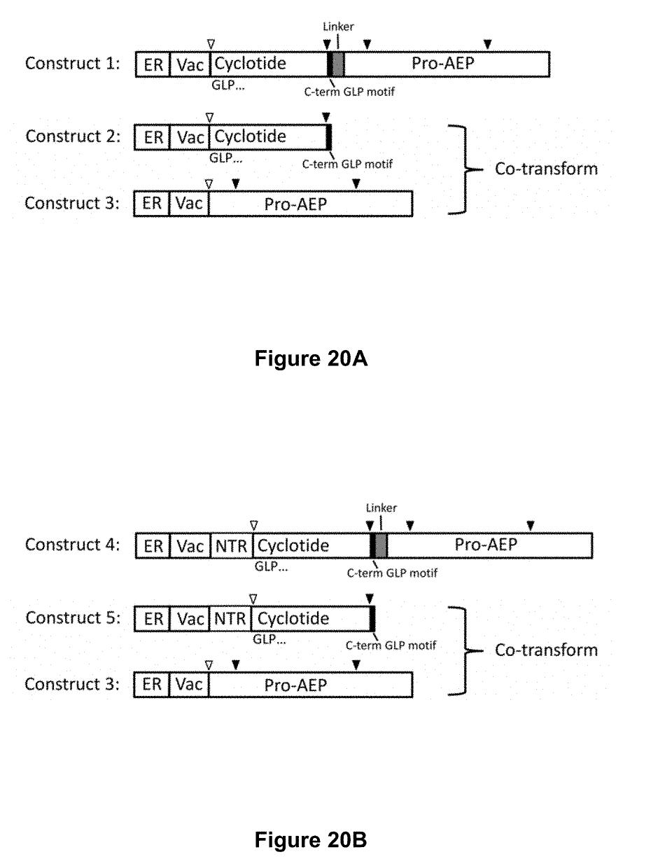

To investigate whether cyclic peptides could be produced in vivo, DNA encoding kalata B1 (mature cyclotide domain Gly₁-Asn₂₉; C-terminal tail, Gly₃₀-Pro₃₂) and/or OaAEP1_{b} (Ala₂₄-Pro₄₇₄) was introduced into Pichia pastoris for co-expression; either from the same or a separate transcriptional unit (FIG. 20A).

For co-expression of kalata B1 and OaAEP1_{b} from the same transcriptional unit, DNA encoding the ER signal sequence together with the vacuolar targeting sequence (VTR) from P. pastoris carboxypeptidase Y (residues Met₁-Val₁₀₇) (Ohi et al. (1996) Yeast 12:31-40), kalata B1 and OaAEP1_{b} were inserted into pPIC9 (FIG. 20A, construct 1). The pPIC9 secretion signal was replaced with the vacuolar targeting signal. Optionally an NTR is included between the VTR and the cyclotide domain (FIG. 20B, construct 4) or residues Met₁-Lys₁₀₈ of the P. pastoris carboxypeptidase Y sequence are included in the construct described above. A linker region (Ala-Ala-Ala-Gly-Gly-Gly-Gly-Gly-Ser—SEQ ID NO:18) was included between kalata B1 and OaAEP1_{b} to reduce steric hindrance between the cyclotide and AEP domains at the protein level and introduce restriction sites for easy substitution of the cyclotide domain with DNA sequences encoding other target peptides. Alternative linkers could incorporate the MGEV linker (Glu-Glu-Lys-Lys-Asn—SEQ ID NO:17) or an extended sequence (e.g.Ala-Ala-Ala-[Gly-Gly-Gly-Gly-Gly-Ser]²⁻⁵). The foreign DNA was then introduced into GS115 P. pastoris cells. The vector encoding kalata B1 and OaAEP1_{b} was then linearized by restriction digestion with SalI and introduced into GS115 cells where it was integrated into the genome at the his4 locus.

Kalata B1 and OaAEP1_{b} were also expressed from separate transcriptional units (FIG. 20 A, constructs 2 and 3). DNA encoding an ER signal sequence and a vacuolar targeting sequence (P. pastoris carboxypeptidase Y, residues Met₁-Val₁₀₇) and kalata B1 (including a short C-terminal tail [Gly-Leu-Fro]) (FIG. 20 A, construct 2) was inserted into pPICZa (such that the alpha mating factor secretion signal was cloned out and replaced with the ER signal sequence and vacuolar targeting sequence). The vector was then linearized with SacI and introduced into GS115 cells where it was integrated into the genome at the 5′ AOX1 locus. Optionally the cyclotide domain is preceded by an NTR inserted C-terminally to the vacuolar targeting sequence (FIG. 20 B, construct 5). DNA encoding an ER signal sequence and a vacuolar targeting sequence (P. pastoris carboxypeptidase Y, residues Met₁-Val₁₀₇) and OaAEP1_{b} (FIG. 20 A, construct 3) was inserted into pPIC9 (such that the alpha mating factor secretion signal was cloned out and replaced with the ER signal sequence and the vacuolar targeting sequence). The vector was then linearized by restriction digestion with SalI and introduced into GS115 cells already harboring the kalata B1 construct. The OaAEP1_{b} construct was integrated into the genome at the his4 locus.

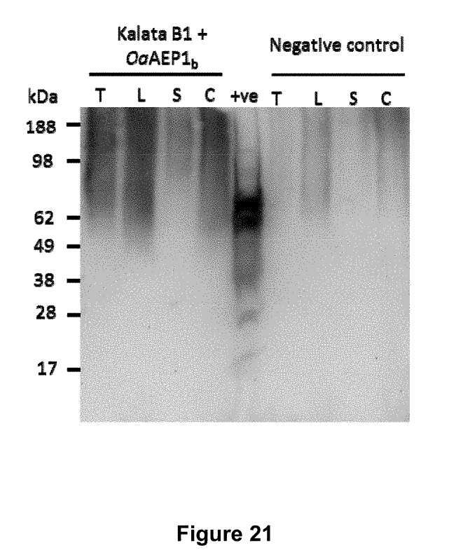

GS115 cells harboring the appropriate construct/s were grown in 5 mL buffered minimal glycerol medium (BMG; 10 mM potassium phosphate, pH 6, 0.34% w/v yeast nitrogen base, 4×10⁻⁵% w/v biotin, 1% v/v glycerol) at 30° C., with shaking, for 48 hours. This starter culture was then used to inoculate 40 mL of BMG and grown at 30° C., with shaking, overnight. Cells were harvested by centrifugation and resuspended in 200 mL buffered methanol medium (BMM; 10 mM potassium phosphate, pH 6, 0.34% w/v yeast nitrogen base, 4×10⁻⁵% w/v biotin, 1% v/v methanol) to induce recombinant protein expression. The culture was incubated at 30° C., with shaking, for 72 hours and methanol was added to 0.5% every 24 hours. After 72 hours, cells were harvested by centrifugation and resuspended in breaking buffer (30 mM HEPES, pH 7.4, 500 mM NaCl) (Visweswaraiah et al. (2011) J. Biol. Chem. 286(42):36568-36579) with an equal volume of glass beads. Cells were disrupted by vigorous agitation using a GenoGrinder (AXT) and soluble material was harvested by centrifugation. Samples were analysed by SDS-PAGE followed by Western blotting (anti-AEP1_{b} rabbit serum [1:2000]; peroxidase-conjugated anti-rabbit IgG [GE Healthcare; 1:5000] (FIG. 21). Expression of OaAEP1_{b} is evident, as judged by antibody reactivity, however the smeared pattern and higher than predicted apparent molecular weight suggests the protein is modified and may be glycosylated or aggregated.

The vacuolar targeting signal is added to facilitate trafficking of the expressed proteins to the vacuole of Pichia pastoris and the in vivo cyclization of target peptides. The cyclic target peptides are then directly purified from the cells. This could be aided by isolation of the vacuolar fraction. This is carried out as previously described (Cabrera and Ungermann (2008) Methods Enzymol 451:177-196). Volumes relate to a 1 L culture at OD₆₀₀ and are scaled accordingly. Thawed cells are resuspended in 33.3 mL 0.1M Tris-HCl, pH 9.4, 10 mM dithiothreitol (DTT) and incubated at 30° C. for 10 minutes. Cells are then harvested by centrifugation and resuspended in 6.7 mL spheroplasting buffer (0.18×YPD [0.18% w/v yeast extract, 0.36% w/v bactopeptone, 0.36% w/v dextrose, pH 5.5], 240 mM sorbitol, 50 mM potassium phosphate pH 7.5). A further 3.3 mL of spheroplasting buffer combined with lyticase (Sigma, as per manufacturer's instructions) is then added and cells are incubated at 30° C., 20 minutes. Cells are harvested by centrifugation and resuspended in 1.67 mL 15% Ficoll (w/v in PS buffer [10 mM PIPES/KOH, pH 6.8, 200 mM sorbitol]). Dextran solution (10 mg mL⁻¹ DEAE-dextran, 10 mM PIPES/KOH, pH 6.8, 200 mM sorbitol) is added to 0.4 mg mL⁻¹ and cells are incubated on ice (5 minutes), 30° C. (1.5 minutes), and ice again (5 minutes). Cell lysates are transferred to centrifuge tubes and sequentially layered with 3 mL of 8% w/v Ficoll (in PS buffer), 4% w/v Ficoll (in PS buffer) and PS buffer. The lysate is centrifuged at 110,000×g at 4° C. for 90 minutes and vacuoles are collected from the 0-4% w/v Ficoll interface.

Isolated vacuoles are osmotically lysed (Wiederhold et al. (2009) Mol Cell Proteomics 8:380-392) by addition of a four-fold volume of 20 mM Tris-HCl, pH 8, 10 mM MgCl₂, 50 mM KCl (30 minutes, 4° C. with agitation). The lysed vacuoles are filtered through a 0.22 μm filter, further diluted 1:4 with 20 mM Tris-HCl, pH 8 and bound to a Q sepharose high performance anion exchange resin (GE Healthcare). Bound kalata B1 is recovered by a continuous salt gradient (0-100% 20 mM Tris-HCl, 1M NaCl pH 8), buffer exchanged into ultrapure water and concentrated. For further purification, the sample is loaded onto an Agilent Zorbax C18 reversed-phase column (4.6×250 mm, 300 Å) and separated using a linear gradient of 5-55% buffer B (90% acetonitrile, 10% v/v H₂O, 0.05% v/v TFA) in buffer A (0.05% v/v TFA/H₂O) over 60 minutes. Fractions containing kalata B1 are lyophilized, resuspended in ultrapure water and analyzed by MALDI MS, HPLC or NMR to confirm its cyclic structure.

As cyclized proteins are generally more stable than linear proteins a crude extract could also be heated to 70° C. for 1 hour after cell disruption and centrifuged at 4000 g for 20 minutes to denature and remove the majority of non-cyclized cellular protein. Cyclized protein will then be purified from the cleared extract as described below for the vacuolar extract.

### Example 9

### Polypeptide Ligation

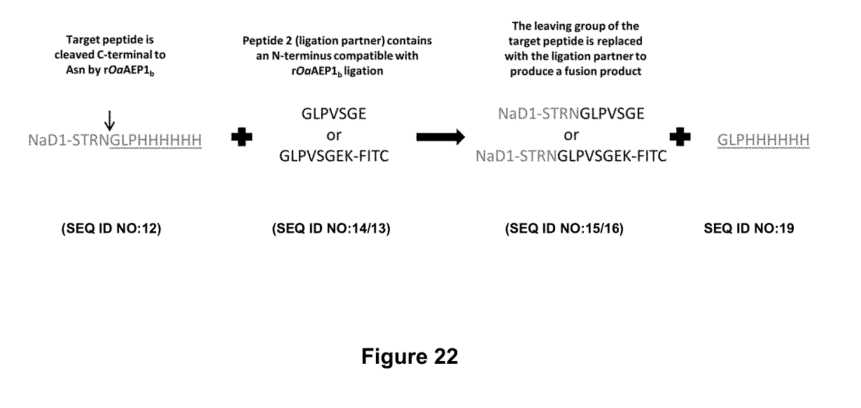

The plant defensin NaD1 (Lay et al. (2003) Plant Physiol 131:1283-1293) with a C-terminal flanking sequence that incorporates an AEP cleavage site and a 6xHis tag (NaD1-STRNGLPHHHHHH—SEQ ID NO:12; 280 μM) is incubated with a ligation partner (GLPVSGEK—SEQ ID NO:13-fluorescein isothiocyanate [FITC] or GLPVSGE; —SEQ ID NO:14-5.6 mM) and rOaAEP1_{b} (12 μg mL⁻¹ total protein) in activity buffer (50 mM sodium acetate, 50 mM NaCl, 1 mM EDTA, 0.5 μM TCEP, pH 5) for 22 hours at room temperature (FIG. 22). The appearance of the ligation product (NaD1-STRNGLPVSGEK-FITC—SEQ ID NO:16 or NaD1-STRNGLPVSGE—SEQ ID NO:15) is tracked by MALDI MS. To separate unprocessed NaD1 from ligated product, the mixture is diluted 1:5 in non-denaturing lysis buffer (without triton X 100) and passed over a Ni-NTA resin (QIAgen). The ligated product does not contain a 6xHis tag and is therefore present in the unbound fraction. This product is then dialyzed into ultrapure water (3 Da molecular weight cut off to ensure the leaving group is also removed), concentrated, and analysed by MALDI MS to confirm the correct ligation product has been generated. Ligation of short, labeled peptides to a larger polypeptide provides a generic, targeted protein labeling strategy for a variety of moieties (e.g. other fluorescent labels, biotin, affinity tags) that is limited only by the ability of synthetic peptide chemistry to produce the appropriate ligation partner.

### Example 10

### Expression and Activation of Other Recombinant AEPs in E. Coli

AEPs from Cicer arietinum (SEQ ID NO: 92), Medicago truncatula (SEQ ID NO: 93), Hordeum vulgare (SEQ ID NO: 94), Gossypium raimondii (SEQ ID NO: 95) and Chenopodium quina (SEQ ID NO: 96) are recombinantly expressed in E. coli. DNA encoding these full-length AEPs without the putative signalling domain (CaAEP residues Q₅₆-P₄₆₀, MtAEP residues E₅₄-N₄₉₇, HvAEP residues G₆₀-Y₅₀₈, GrAEP residues Q₃₁-H₅₀₀, CqAEP residues R₃₃-V₅₉₉) is inserted into the pHUE vector (Catanzariti et al. (2004) supra) to give a 6xHis-ubiquitin-AEP fusion protein construct. Residue numbering is as determined by a multiple alignment of the five sequences generated using Clustal Omega (Sievers et al. (2011) supra). DNA is then introduced into T7 Shuffle E. coli cells (New England BioLabs). Transformed cells are grown at 30° C. in superbroth (3.5% tryptone [w/v], 2% yeast extract [w/v], 1% glucose [w/v], 90 mM NaCl, 5 mM NaOH) to mid-log phase; the temperature is then reduced to 16° C. and expression is induced with isopropyl ß-D-1-thiogalactopyranoside (IPTG; 0.4 mM; Bio Vectra) for approximately 20 hours. Cells are harvested by centrifugation and resuspended in non-denaturing lysis buffer (50 mM Tris-HCl, 150 mM NaCl, 0.1% triton X 100, 1 mM EDTA, pH 7). Lysis is promoted by a total of five freeze/thaw cycles and the addition of lysozyme (hen egg white; Roche; 0.4 mg mL⁻¹). DNase (bovine pancreas; Roche; 40 μg mL⁻¹) and MgCl₂ (0.4 M) are also added. Cellular debris is removed by centrifugation and the lysate is stored at −80° C. until required.

Lysate containing expressed recombinant AEPs is filtered through a 0.1 μM glass fibre filter (GE Healthcare) before being diluted 1:8 in buffer A (20 mM bis-Tris, 0.2 M NaCl, pH 7) and loaded onto two 5 mL HiTrap Q Sepharose high performance columns connected in series (GE Healthcare; 1.6-3.1 mL undiluted lysate mL⁻¹ resin). Bound proteins are eluted with a continuous salt gradient (0-30% buffer B [20 mM bis-Tris, 2 M NaCl, pH 7]; 15 column volumes [cv]) and AEP-positive fractions are identified by Western blotting (anti-AEP1_{b} rabbit serum [1:2000]; peroxidase-conjugated anti-rabbit IgG [GE Healthcare; 1:5000]).

AEPs are usually produced as zymogens that are self-processed at low pH to their mature, active form (Hiraiwa et al. (1997) supra; Hiraiwa et al. (1999) supra; Kuroyanagi et al. (2002) supra). To self-activate all AEPs, EDTA (1 mM) and TCEP (Sigma-Aldrich; 0.5 mM) are added, the pH is adjusted to 4.5 with glacial acetic acid and the protein pool is incubated for 5 hours at 37° C. Protein precipitation at pH 4.5 allows removal of the bulk of the contaminating proteins by centrifugation. The remaining protein is filtered (0.22 μm; Millipore), diluted 1:8 in buffer A2 (50 mM acetate, pH 4) then captured on a 1 mL HiTrap SP Sepharose high performance column (GE Healthcare). Bound proteins are eluted with a salt gradient (0-100% buffer B2 [50 mM acetate, 1 M NaCl, pH 4]; 10 cv) and fractions with activity against an IQF peptide (Abz-STRNGLPS-Y(3NO₂) or other target sequence or fluorescent peptide as appropriate) are pooled and used in subsequent activity assays. The total concentration of protein in each preparation is estimated by BCA assay according to the manufacturer's instructions. Enzymes are used in cyclization and ligation assays as described in the Materials and Methods.

Those skilled in the art will appreciate that aspects of aspects described herein are susceptible to variations and modifications other than those specifically described. It is to be understood that these aspects include all such variations and modifications. These aspects also include all of the steps, features, compositions and compounds referred to or indicated in this specification, individually or collectively, and any and all combinations of any two or more of the steps or features.

### BIBLIOGRAPHY

Altschul et al. (1997) Nucl Acids Res 25: 3389-3402
Altschul et al. (1990) J Mol Biol 215: 403-410
Arnison et al. (2013) Nat Prod Rep 30:108-160
Ausubel et al. (In: Current Protocols in Molecular Biology, John Wiley & Sons Inc. 1994-1998
Barber et al. (2013) J. Biol. Chem 288:12500-12510
Bernath-Leven et al. (2015) Chemistry & Biology 22:1-12
Cabrera and Ungermann (2008) Methods Enzymol 451:177-196
Camarero et al. (2001) Bioorganic Med Chem 9:2479-2484
Catanzariti et al. (2004) Protein Sci 13:1331-1339
Chan et al. (2013) Chembiochem 14:617-624
Clark et al. (2005) Proc. Natl. Acad. Sci. United States Am. 102:13767-13772
Clark et al. (2010) Angew. Chem. Int. Ed. Engl. 49:6545-6548
Colgrave et al. (2008) Biochemistry 47:5581-5589
Colgrave et al. (2009) Acta Trop. 109:163-166
Dall et al. (2015) Angewandte Chemie (International Ed. in English) 54: 2917-2921
Gillon et al. (2008) Plant J. 53:505-515
Goransson et al. (2004) J. Nat. Prod. 67:1287-1290
Gran (1973) Acta Pharmacol. Toxicol. 33:400-408
Gustafson et al. (2000) J. Nat. Prod 63:176-178
Hanada et al. (2004) Nature 427:252-256
Harlow and Lane (1988) Antibodies: A Laboratory Manual, Cold Spring Harbor Laboratories.
Harris et al. (2005) Infect. Immun. 73:6981-6989
Harris et al. (2009) J. Biol. Chem. 284:9361-9371
Hatsugai et al. (2004) Science 305(5685): 855-858
Hiraiwa et al. (1997) Plant J 12(4):819-829
Hiraiwa et al. (1999) FEBS Lett 447(2-3):213-216
Jennings et al. (2001) Proc. Natl. Acad. Sci. U.S.A 98:10614-10619
Kuroyanagi et al. (2002) Plant Cell Physiol 43(2):143-151
Lay et al. (2003) Plant Physiol 131:1283-129
Lay et al. (2012) J Biol Chem 287:19961-19972
Lee et al. (2009) J. Am. Chem. Soc. 131:2122-2124
Li et al. (2006) Bioinformatics 22: 1658-1659
Li et al. (2009) Bioinformatics 25: 1754-1760
Lindholm et al. (2002) Mol. Cancer Ther. 1:365-369
Luo et al. (2014) Chem. Biol. 1-8 doi:10.1016/j.chembiol.2014.10.015
Mazmanian et al. (1999) Science (80) 285:760-763
Mylne et al. (2011) Nat. Chem. Biol. 7:257-925
Mylne et al. (2012) Plant Cell 24:2765-2778
Nguyen et al. (2014) Nat. Chem. Biol. 10:732-738
Nilsson et al. (1989) Cell 58:707-718
Nolde et al. (2011) J Biol Chem 286(28):25145-25153
Ohi et al. (1996) Yeast 12:31-40
Plan et al. (2008) J. Agric. Food Chem. 56:5237-5241
Poth et al. (2013) Biopolymers 100:480-491
Qin et al. (2010) BMC Genomics 11: 111
Quimbar et al. (2013) J Biol Chem 288(19):13885-13896
Rotari et al. (2001) Biol. Chem. 382:953-959
Sambrook et al. (1989) Molecular Cloning, Second Edition, Cold Spring Harbor Laboratory, Plainview, N.Y.
Saska et al. (2008) Journal of Chromatography B. 872:107-114
Saska et al. (2007) J. Biol. Chem. 282:29721-29728
Schulz et al. (2012) Bioinformatics 28: 1086-1096
Sheldon et al. (1996) Biochem. J. 320:865-870
Sievers et al. (2011) Mol. Syst. Biol 7: 539
Simonsen et al. (2004) FEBS Lett. 577:399-402
Tam et al. (1999) Proc. Natl. Acad. Sci. U.S.A 96:8913-8918
Visweswaraiah et al. (2011) J. Biol. Chem. 286(42):36568-36579
Wiederhold et al. (2009) Mol Cell Proteomics 8:380-392
Witherup et al. (1994) J. Nat. Prod 57:1619-1625
Wu and Hancock (1999) Antimicrob Agents Ch 43:1274-1276

1. A method for producing a cyclic peptide said method comprising generating a recombinant asparaginyl endopeptidase (AEP) vacuolar processing enzyme with peptide cyclization activity in a prokaryotic or eukaryotic cell and co-incubating the AEP with a linear polypeptide precursor of the cyclic peptide wherein the polypeptide precursor comprises N-terminal and/or C-terminal AEP processing site(s) for a time and under conditions sufficient to generate the cyclic peptide.

2. The method of claim 1 comprising introducing into the cell genetic material which, when expressed, generates the linear polypeptide precursor wherein the cell is incubated for a time and under conditions sufficient to generate a cyclic peptide in vivo and then isolating the cyclic peptide.

3. The method of claim 1 wherein the recombinant AEP is co-incubated with a linear polypeptide precursor or a post-translationally or synthetically modified form thereof in vitro in a reaction vessel for a time and under conditions sufficient to generate the cyclic peptide.

4. The method of claim 1 for producing a cyclic peptide said method comprising introducing an expression vector into a prokaryotic or eukaryotic cell encoding the linear polypeptide precursor, enabling expression of the vector to produce a recombinant linear polypeptide precursor and isolating the polypeptide from the cell and co-incubating in a reaction vessel the polypeptide precursor with recombinant AEP for a time and under conditions sufficient to generate the cyclic peptide.

5-6. (canceled)

7. A method for generating a peptide conjugate, said method comprising co-incubating at least two peptides wherein at least one peptide comprises a C-terminal AEP recognition amino acid sequence and at least one other peptide comprises an N-terminal AEP recognition amino acid sequence with an AEP for a time and under conditions sufficient to generate a linear peptide conjugate.

8. The method claim 1 wherein the polypeptide precursor is in the form of multiple repeats of the peptide to be cyclized or is in the form of multiple different polypeptides to be cyclized.

9. A method of claim 1 wherein the AEP comprises an amino acid sequence having at least 80% similarity to any one or more of SEQ ID NOs:1, 2 and/or 4 after optimal alignment and the presence of 5 or more of residues or absence of residues at 139K, 161D, 186K, 192D, 247C, 248Y, 253Q, 255A, 263V, 293H, Gap, Gap, Gap, Gap, Gap (between residues 299 and 300), 314E and 316G wherein Gap means the absence of a residue.

10. The method of claim 1 comprising introducing one or more expression vectors into a prokaryotic or eukaryotic cell encoding the AEP and the polypeptide precursor, enabling expression of the vector to produce a recombinant AEP and a recombinant linear polypeptide precursor and isolating a cyclic peptide from the appropriate compartment or expression medium of the eukaryotic or prokaryotic cell wherein the expression vector is a multi-gene expression vehicle consisting of a polynucleotide comprising from 2 or more transcription segments, each segment encoding the AEP or linear polypeptide precursor, each segment being joined to the next in a linear sequence by a linker segment encoding a linker peptide, the transcription segments all being in the same reading frame operably linked to a single promoter and terminator.

11-12. (canceled)

13. The method of claim 1 wherein the cell is E. coli or a yeast wherein the yeast is Pichia spp., Saccharomyces spp. or Kluyveromyces spp.

14. (canceled)

15. The method of claim 1 wherein the cyclic peptide exhibits antipathogenic or therapeutic properties including for the treatment of infection or infestation by a pathogen or treatment of cancer, cardiovascular disease, immune disease and pain.

16. (canceled)

17. The method of claim 1 wherein the C-terminal AEP processing site comprises P3 to P1 prior to the actual cleavage site and comprising P1′ to P3′ after the cleavage site towards the C-terminal ends wherein P3 to P1 and P1 to P3 have the amino acid sequence:
X₂X₃X₄X₅X₆X₇
wherein X is an amino acid residue and:
X₂ is optional or is any amino acid;
X₃ is optional or is any amino acid;
X₄ is N or D;
X₅ is G or S;
X₆ is L or A or I; and
X₇ is optional or any amino acid;
and/or wherein the N-terminal processing site may contain no specific AEP processing site or may contain a processing site defined by any one of P1″ through P3″ wherein P1″ to P3″ is defined by:
X₉X₁₀X₁₁
wherein X is an amino acid residue:
X₉ is optional and any amino acid or G, Q, K, V or L;
X₁₀ is optional or any amino acid or L, F or I or an hydrophobic amino acid residue;
X₁₁ is optional and any amino acid.

18. The method of claim 17 wherein X₂ through X₇ comprise the amino acid sequence:
X₂X₃NGLX₇
wherein X₂, X₃ and X₇ are as defined in claim 17; and
wherein X₉ through X₁₁ comprise the amino acid sequence:
GLX₁₁
wherein X₁₁ is optional and any amino acid.

19-20. (canceled)

21. The method of claim 1 wherein the AEP processing site comprises N- and C-terminal end sequences comprising the sequence:
G_{LX11} [Xₙ] X₂X₃NGLX₇
wherein X₁₁, X₂, X₃, and X₇ are optional and any amino acid and [Xₙ] is absent (n=0) or any amino acid residue in a sequence of from 1 to 2000 amino acids.

22. A method for enzymatic transpeptidation involving cleavage of an amide bond, said method comprising co-incubating a polypeptide precursor with an asparaginyl endopeptidase (AEP) wherein the amide bond cleavage is coupled to formation of a new amide bond wherein C- and N-termini of the polypeptide precursor are enzymatically ligated to produce a circular peptide or wherein the C- and N-termini of at least two separate polypeptides are ligated to produce a new linear polypeptide.

23-34. (canceled)

35. The method of claim 22 wherein the AEP is co-expressed with the polypeptide precursor and incubated for a time and under conditions sufficient for cyclization or ligation to occur in vivo.

36-38. (canceled)

39. The method of claim 22 wherein the AEP and polypeptide precursor are expressed in a multi-gene expression vehicle or wherein the AEP and polypeptide precursor are expressed in different vectors.

40-43. (canceled)

44. The method of claim 22 wherein the AEP comprises an amino acid sequence having at least 80% similarity to any one or more of SEQ ID NOs:1, 2 and/or 4 after optimal alignment and wherein the presence of 5 or more of residues or absence of residues at 139K, 161D, 186K, 192D, 247C, 248Y, 253Q, 255A, 263V, 293H, Gap, Gap, Gap, Gap, Gap (between residues 299 and 300), 314E and 316G wherein Gap means the absence of a residue.

45-46. (canceled)

47. The method of claim 22 wherein the cell is E. coli or a yeast wherein the yeast is Pichia sp., Saccharomyces sp. or Kluyveromyces sp.

48-50. (canceled)

51. The method of claim 15 wherein the AEP and polypeptide precursor are targeted to a periplasmic space or a vacuole.

52-54. (canceled)

55. The method of claim 22 wherein the cyclic peptide comprises a functional portion fused or embedded in a backbone framework of a cyclotide.

56-57. (canceled)

58. An agronomical composition or pharmaceutical composotion comprising the cyclic peptide generated by the method of claim 1 or 22.

59.-63. (canceled)

64. A method for identifying an AEP with cyclizing ability, said method comprising co-incubating an AEP to be tested with an internally-quenched fluorescent (IQF) peptide and assaying for a change in fluorescent intensity over time due to fluorescence upon spatial separation of a fluorescence donor/quencher pair following enzymatic cleavage of the peptide wherein an elevation in the of fluorescent intensity is indicative of an AEP with cyclizing ability wherein fluorescence intensity is monitored over time at excitation/emission wavelengths 320/420 nm.

65. The method of claim 64 wherein the IQF peptide is selected from the group consisting of Abz-STRNGLPS-Y(3NO₂) [SEQ ID NO:21] and Abz-STRNGAPS-Y(3NO₂) [SEQ ID NO:25].

66-67. (canceled)

68. A method for determining whether an AEP is likely to have cyclization activity, said method comprising determining the amino acid sequence of the AEP, aligning the sequence with a best fit to the amino acid sequence of OaAEP1_{b} (SEQ ID NO:1) and screening for the presence of 5 or more of residues or absence of residues at 180K, 219D, 274K, 280D, 352C, 353Y, 359Q, 361A, 379V, 506H, 519Gap, 520Gap, 521Gap, 525Cap, 526Gap, 542E and 544G wherein gap means the absence of a residue wherein the presence of 5 or more of the listed residues or absence of residues is indicative of an AEP which is a cyclase.


SEQUENCE LISTING (104 sequences)

SEQ ID NO: 1 - protein, 474 aa, artificial
  Oldenlandia affinis OaAEP1b
MVRYLAGAVLLLVVLSVAAAVSGARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEVAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIP

SEQ ID NO: 2 - protein, 474 aa, artificial
  Oldenlandia affinis OaAEP1
MVRYLAGAVLLLVVLSVAAAVSGARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEEAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIP

SEQ ID NO: 3 - protein, 488 aa, artificial
  Oldenlandia affinis OaAEP2
MVRYPAGAVLLLVVLSVVAVDGARDGYLKLPSEVSDFFRPRNTNDGDDSVGTRWAVLLAGSNGYWNYRHQADLCHAYQILKRGGLKDENIVVFMYDDIAYNEENPRPGVIINSPHGSDVYAGVPKDYTGDQVNAKNFLAAILGNKSAITGGSGKVVNSGPNDHIFIYYTDHGGPGVLGMPVGPYIYADDLIDTLKKKHASGTYKSLVFYLEACESGSMFEGLLPEGLNIYATTASNAEESSWGTYCPGEYPSPPPEYDTCLGDLYSVAWMEDSEVHNLRSETLKQQYHLVKARTSNGNSAYGSHVMQYGDLKLSVDNLFLYMGTNPANDNYTFVDDNALRPSSKAVNQRDADLLHFWDKFRKAPEGSARKEEARKQVFEAMSHRMHIDNSIKLVGKLLFGIERGAEILDAVRPAGQPLADDWTCLKSLVRTFETHCGSLSQYGMKHMRTIANICNAGITKEQMAEASAQACSSVPSNPWSSLHKGFSA

SEQ ID NO: 4 - protein, 489 aa, artificial
  Oldenlandia affinis OaAEP3
MVRYLAGAFQVVLLVVILSDIAISEERTDGYLKLPTEVSRFFRTPEQSSDGGDDSIGTRWAVLIAGSKGYDNYRHQADVCHAYQILKRGGLKDENIVVFMYDDIAYNESNPRPGVIINSPHGSDVYAGVPKDYTGDEVNAKNFLAAILGNKSAITGGSGKVVDSGPNDHIFIYYTDHGAPGVIGMPSKPYLYADELNDALRKKHASGTYKSMVFYLEACEAGSMFDGLLPDGLNIYALTASNTTEGSWCYYCPGQDAGPPPEYSVCLGDFFSIAWLEDSDVHNLRSETLNQQYHNVKNRISYASHATQYGDLKRGVEGLFLYLGSNPENDNYTFVDDNVVRPSSKAVNQRDADLVHFWEKFRKAPEGSSKKEEAQKQILEAMSHRVHIDSSINLIGKLLFGIEKGHKILTAVRSAGHPLVDDWACLRSLVRTFETHCGSLSQYGMKHTRTLANICNAGITEEQMAEAASQACVSIPSNPWSSHDGGFSA

SEQ ID NO: 5 - protein, 20 aa, artificial
  Amino acid sequence of model peptide with flanking sequences
VFAEFLPLFSKFGSRMHILK

SEQ ID NO: 6 - DNA, 17 bp, artificial
  OaAEPdegen-F, 5′ forward primer
gttcgatatcycgccgg

SEQ ID NO: 7 - DNA, 29 bp, artificial
  OaAEP1-R, 5′ reverse primer
tcatgaactaaatcctccatggaaagagc

SEQ ID NO: 8 - DNA, 26 bp, artificial
  OaAEP2-R, 5′ reverse primer
ttatgcactgaatcctttatggaggg

SEQ ID NO: 9 - DNA, 22 bp, artificial
  OaAEP3-R, 5′ reverse primer
ttatgcactgaatcctccatcg

SEQ ID NO: 10 - protein, 7 aa, artificial
  C-terminal pro-hepta-peptide
GLPSLAA

SEQ ID NO: 11 - protein, 36 aa, artificial
  kB1wt
GLPVCGETCVGGTCNTPGCTCSWPVCTRNGLPSLAA

SEQ ID NO: 12 - protein, 13 aa, artificial
  C-terminal flanking sequence for NaD1
STRNGLPHHHHHH

SEQ ID NO: 13 - protein, 8 aa, artificial
  Ligation partner
GLPVSGEK

SEQ ID NO: 14 - protein, 7 aa, artificial
  Ligation partner
GLPVSGE

SEQ ID NO: 15 - protein, 58 aa, artificial
  Ligation product
RECKTESNTFPGICITKPPCRKACISEKFTDGHCSKILRRCLCTKPCSTRNGLPVSGE

SEQ ID NO: 16 - protein, 58 aa, artificial; identical to SEQ ID NO: 15
  Ligation product

SEQ ID NO: 17 - protein, 5 aa, artificial
  Linker
EEKKN

SEQ ID NO: 18 - protein, 9 aa, artificial
  Linker
AAAGGGGGS

SEQ ID NO: 19 - protein, 9 aa, artificial
  Target peptide
GLPHHHHHH

SEQ ID NO: 20 - protein, 534 aa, artificial
  6xHis-ubiquitin-OaAEP1b fusion protein
MHHHHHHMQIFVKTLTGKTITLEVEPSDTIENVKAKIQDKEGIPPDQQRLIFAGKQLEDGRTLSDYNIQKESTLHLVLRLRGGARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEVAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIP

SEQ ID NO: 21 - protein, 8 aa, artificial
  Internally quenched peptide
STRNGLPS

SEQ ID NO: 22 - DNA, 375 bp, artificial
  Nucleotide sequence encoding full kalata B1 protein
atggctaagttcaccgtctgtctcctcctgtgcttgcttcttgcagcatttgttggggcgtttggatctgagctttctgactcccacaagaccaccttggtcaatgaaatcgctgagaagatgctacaaagaaagatattggatggggtggaagctactttggtcactgatgtcgccgagaagatgttcctaagaaagatgaaggctgaagcgaaaacttctgaaaccgccgatcaggtgttcctgaaacagttgcagctcaaaggacttccagtatgcggtgagacttgtgttgggggaacttgcaacactccaggctgcacttgctcctggcctgtttgcacacgcaatggccttcctagtttggccgcataa

SEQ ID NO: 23 - protein, 124 aa, artificial
  Amino acid sequence of full kolata B1 protein
  MISC_FEATURE (1)..(20): Signal sequence
  MISC_FEATURE (21)..(66): N-terminal prddomain
  MISC_FEATURE (67)..(88): N-terminal repeat
  MISC_FEATURE (89)..(117): Mature cyclotide sequence
  MISC_FEATURE (118)..(124): C-terminal prodomain
MAKFTVCLLLCLLLAAFVGAFGSELSDSHKTTLVNEIAEKMLQRKILDGVEATLVTDVAEKMFLRKMKAEAKTSETADQVFLKQLQLKGLPVCGETCVGGTCNTPGCTCSWPVCTRNGLPSLAA

SEQ ID NO: 24 - protein, 12 aa, artificial
  Model peptide Bac2A
RLARIVVIRVAR

SEQ ID NO: 25 - protein, 8 aa, artificial
  Internally quenched peptide L31A
STRNGAPS

SEQ ID NO: 26 - protein, 30 aa, artificial
  rOaAEP1b-mediated processing
GLPVFAEFLPLFSKFGSRMHILKSTRNGLP

SEQ ID NO: 27 - protein, 25 aa, artificial
  rOaAEP1b-mediated processing
GLVFAEFLPLFSKFGSRMHILKNGL

SEQ ID NO: 28 - protein, 24 aa, artificial
  rOaAEP1b-mediated processing
GLVFAEFLPLFSKFGSRMHILKNG

SEQ ID NO: 29 - protein, 23 aa, artificial
  rOaAEP1b-mediated processing
VFAEFLPLFSKFGSRMHILKNGL

SEQ ID NO: 30 - protein, 25 aa, artificial
  rOaAEP1b-mediated processing
QLVFAEFLPLFSKFGSRMHILKNGL

SEQ ID NO: 31 - protein, 25 aa, artificial
  rOaAEP1b-mediated processing
KLVFAEFLPLFSKFGSRMHILKNGL

SEQ ID NO: 32 - protein, 8 aa, artificial
  C-terminal AEP recognition
  MISC_FEATURE (1)..(3): optional or any amino acid
  MISC_FEATURE (4)..(4): N or D
  MISC_FEATURE (5)..(5): G or S
  MISC_FEATURE (6)..(6): L or A or I
  MISC_FEATURE (7)..(8): optional or any amino acid
  MISC_FEATURE (7)..(8): optional or any amino acid
XXXXXXXX

SEQ ID NO: 33 - protein, 4 aa, artificial
  N-terminal AEP recognition
  MISC_FEATURE (1)..(1): optional and any amino acid or G, Q, K, V or L
  MISC_FEATURE (2)..(2): optional and any amino acid or L, F or I or a hydrophobic amino acid residue
  MISC_FEATURE (3)..(4): optional and any amino acid
XXXX

SEQ ID NO: 34 - protein, 8 aa, artificial
  C-terminal AEP recognition
  MISC_FEATURE (1)..(3): optional or is any amino acid
  MISC_FEATURE (7)..(8): optional or is any amino acid
XXXNGLXX

SEQ ID NO: 35 - DNA, 1392 bp, artificial
  OaAEP1b
ggatccatggttcggtatctcgccggagcagtcctactcctagttgtactttcagttgccgccgccgtatccggagctcgtgatggcgactatctacatctgccatcggaagtttcccgatttttccggccacaggagaccaacgacgaccacggcgaagactcggtcggaactagatgggctgtcctgatcgctgggtcgaaaggttatgcaaactaccggcatcaggctggtgtttgtcatgcatatcaaatattgaaaagaggaggtcttaaagatgaaaacattgtggtattcatgtatgacgacattgcctacaatgaatcgaaccctaggcctggagttatcatcaacagcccacacggcagtgatgtttatgccggagtcccaaaggattatacaggggaagaggttaatgctaagaactttttggcagctattcttggcaacaagtctgctattacggggggtagcggcaaggtggttgatagtggtccaaatgatcacatcttcatctactatacagatcacggtgccgctggggtaattgggatgccttcaaaaccttacctttatgcggatgaattaaatgatgctttgaagaagaagcatgcttctgggacatataagagcttggtgttttacctggaagcttgtgagtcgggtagcatgtttgagggaatactccctgaggatcttaatatctacgcgctaacatctacaaacacaacagaaagcagttggtgttattattgccctgcacaggaaaatccccctcccccggaatataacgtttgcttgggtgacttatttagtgttgcgtggttggaagacagtgacgtacaaaattcgtggtatgaaactttgaaccagcaatatcaccatgttgacaagagaatctcgcatgcctcccatgccacgcaatatggaaatttgaagctgggtgaggaaggtctattcgtctatatgggttctaaccctgctaatgataattacacttctttggatggcaatgctcttactccatcttcaatagttgttaatcagcgtgatgctgatttattgcacttgtgggaaaagttccgtaaggctcctgaaggctctgcaaggaaagaagtagctcaaacacagatctttaaagcgatgtcccatcgagtgcacatcgacagcagcataaaattaattggaaagcttctctttggtattgagaaatgcactgaaattcttaatgctgtcaggccagctggtcagcctcttgttgatgactgggcctgcctcagatctttggtcggaacatttgagacacattgtggctcgctgtcggaatatggaatgagacatactcggaccattgcaaatatctgcaatgctggaatctctgaggaacagatg

SEQ ID NO: 36 - DNA, 1425 bp, artificial
  OaAEP1
atggttcgatatctcgccggagcagtcctactcctagttgtactttcagttgccgccgccgtatccggagctcgtgatggcgactatctacatctgccatcggaagtttcccgatttttccggccacaggagaccaacgacgaccacggcgaagactcggtcggaactagatgggctgtcctgatcgctgggtcgaaaggttatgcaaactaccggcatcaggctggtgtttgtcatgcatatcaaatattgaaaagaggaggtcttaaagatgaaaacattgtggtattcatgtatgacgacattgcctacaatgaatcgaaccctaggcctggagttatcatcaacagcccacacggcagtgatgtttatgccggagtcccaaaggattatacaggggaagaggttaatgctaagaactttttggcagctattcttggcaacaagtctgctattacggggggtagcggcaaggtggttgatagtggtccaaatgatcacatcttcatctactatacagatcacggtgccgctggggtaattgggatgccttcaaaaccttacctttatgcggatgaattaaatgatgctttgaagaagaagcatgcttctgggacatataagagcttggtgttttacctggaagcttgtgagtcgggtagcatgtttgagggaatactccctgaggatcttaatatctacgcgctaacatctacaaacacaacagaaagcagttggtgttattattgccctgcacaggaaaatccccctcccccggaatataacgtttgcttgggtgacttatttagtgttgcgtggttggaagacagtgacgtacaaaattcgtggtatgaaactttgaaccagcaatatcaccatgttgacaagagaatctcgcatgcctcccatgccacgcaatatggaaatttgaagctgggtgaggaaggtctattcgtctatatgggttctaaccctgctaatgataattacacttctttggatggcaatgctcttactccatcttcaatagttgttaatcagcgtgatgctgatttattgcacttgtgggaaaagttccgtaaggctcctgaaggctctgcaaggaaagaagaagctcaaacacagatctttaaagcgatgtcccatcgagtgcacatcgacagcagcataaaattaattggaaagcttctctttggtattgagaaatgcactgaaattcttaatgctgtcaggccagctggtcagcctcttgttgatgactgggcctgcctcagatctttggtcggaacatttgagacacattgtggctcgctgtcggaatatggaatgagacatactcggaccattgcaaatatctgcaatgctggaatctctgaggaacagatggcggaggcagcctcgcaggcttgtgctagtattccttga

SEQ ID NO: 37 - DNA, 1467 bp, artificial
  OaAEP2
atggttcgatatctcgccggagcagtcctactcctcgtcgtactttcagtcgtcgccgtagatggagcacgtgacggctacctaaaacttccctcggaagtctccgattttttccgacctaggaatacgaacgacggcgacgactctgtcggaactagatgggctgtcctgctcgccggatcgaacggttattggaattaccggcatcaggctgatttatgtcatgcatatcaaatactgaaaagaggaggtctgaaggatgaaaacattgtggtgttcatgtacgatgacattgcctacaatgaagagaaccctaggcctggagttatcatcaacagcccacacggcagtgatgtttatgcaggagtccctaaggattatacaggggatcaagttaatgcgaaaaactttttagcggctatccttggcaacaaatcagctataacggggggtagcggtaaggtggttaatagtggtccaaatgatcacatattcatctactatacagatcatggtggtcctggagttcttgggatgcctgtggggccttacatctatgcggatgatctgattgatactttgaagaagaagcatgcttcagggacatataagagcttggtgttttacctggaagcttgtgagtctggtagcatgtttgagggactacttcctgaaggtctcaatatctatgcaaccacagcctcaaatgcagaggaaagcagttggggaacctattgtccaggagagtatcctagccctcccccagaatatgatacatgcttgggtgacctatatagtgttgcttggatggaagacagtgaggtacacaatttgcggtctgaaactttgaagcagcaatatcacctggttaaagcgagaacctcaaatggtaattcagcttatggctcccatgtcatgcaatatggtgatttgaagctgagtgtggacaatcttttcctctatatgggtactaaccctgcaaatgataattacacttttgtggatgacaatgctcttcgtccatcttcaaaagctgttaatcagcgtgatgctgatttattgcatttctgggacaagttccgtaaggctcctgaaggttctgcaagaaaagaagaagctcgcaaacaggtttttgaagctatgtcccaccggatgcacattgacaacagcatcaaattagttggaaagcttctctttggtattgagagaggcgctgaaattcttgatgctgtcaggccagccggtcagcctctggctgatgactggacctgcctcaaatctttggtcagaacatttgagacacattgtggctcgttgtcgcagtatggaatgaagcatatgcggaccattgctaatatctgcaatgctggaatcacgaaggaacagatggcggaggcatctgcgcaggcatgttccagtgttccttcaaatccttggagctccctccataaaggattcagtgcataa

SEQ ID NO: 38 - DNA, 1470 bp, artificial
  OaAEP3
atggttcgatatctcgccggagcattccaagtagtactcctcgtcgtcatactttcagacatcgccatatctgaagaacgtactgatggctacctaaagctgccgacggaagtttcccggtttttccgtactcctgagcagtcgagcgacggcggtgatgactctattggaactagatgggctgtcctgatcgccggatccaaaggttatgacaactaccggcatcaggctgatgtctgtcatgcatatcaaatcctgaaaagaggaggccttaaagatgagaacattgtagtattcatgtatgatgacattgcctacaatgaatcgaacccgaggcctggagtaataatcaacagcccacacggcagtgatgtttatgccggagtcccaaaggattatacaggggatgaggttaatgctaagaactttttagcagctattcttggcaacaagtcagctattactgggggtagcggcaaggtggttgatagcggtccaaatgatcacattttcatctactatacagatcatggtgctcctggggtcattgggatgccttcgaaaccttacctctacgcggatgaattgaatgatgctttgaggaagaagcatgcttctggaacatataagagcatggtgttttacctggaagcttgtgaggcgggtagcatgtttgacggactacttcctgacggtctcaatatctacgcgctgacagcctcaaacacaacagaaggcagttggtgctattattgccctggacaggatgctggccctcccccagaatacagtgtttgcttgggtgacttttttagtattgcttggttggaagacagtgacgtacacaatttgcggtctgaaactttgaaccagcaatatcacaatgttaagaacagaatctcatatgcctcccatgccacgcaatatggtgatttgaagcgcggtgttgaaggccttttcctctatttaggttctaacccggaaaatgataattacacttttgtggatgacaatgtggttcgtccatcttccaaagctgttaatcagcgtgacgctgatttagtgcacttctgggaaaagtttcgtaaggctcctgaaggttcttcgaagaaagaagaagctcaaaaacagatccttgaagctatgtcccatcgagtgcacattgacagcagcataaatttaattggaaagcttctctttggtattgagaaaggccacaaaattcttactgctgtccggtcagccggccaccctcttgttgatgactgggcctgcctcagatctttggttagaacatttgagacacattgtggctcgctgtcgcagtatggaatgaaacatactcggacacttgcaaatatttgcaatgctggaatcactgaggaacagatggcggaggcagcctcgcaggcctgtgtcagtattccttcaaatccttggagctctcacgatggaggattcagtgcataa

SEQ ID NO: 39 - protein, 483 aa, artificial
  OaAEP4 aa
MVRYPAGAVLLLVVLSVVAVDGARDGYLKLPSEVSDFFRPRNTNDGDDSVGTRWAVLLAGSNGYWNYRHQADLCHAYQILKRGGLKDENIVVFMYDDIAYNEENPRPGVIINSPHGSDVYAGVPKDYTGDEVNAKNFLAAILGNKSAITGGSGKVVDSGPNDHIFIYYTDHGAPGVIGMPSKPYLYADELNDALRKKHASGTYKSMVFYLEACEAGSMFDGLLPDGLNIYALTASNTTEGSWCYYCPGQDAGPPPEYSVCLGDFFSIAWLEDSDVHNLRSETLNQQYHNVKNRISYASHATQYGDLKRGVEGLFLYLGSNPENDNYTFVDDNVVRPSSKAVNQRDADLVHFWEKFRKAPEGSSKKEEAQKQILEAMSHRVHIDSSINLIGKLLFGIEKGHKILTAVRSAGHPLVDDWACLRSLVRTFETHCGSLSQYGMKHTRTLANICNAGITEEQMAEAASQACVSIPSNPWSSHDGGFSA

SEQ ID NO: 40 - DNA, 1386 bp, artificial
  OaAEP4 na
gcccgtgatggctatctgaaactgccgtccgaagtgagcgatttcttccgtccgcgtaataccaacgatggcgatgactccgtgggtacccgttgggcagtgctgctggctggcagcaacggttattggaattaccgtcatcaggcagatctgtgccacgcttatcaaattctgaaacgcggcggtctgaaagacgaaaacatcgtggttttcatgtacgatgacatcgcgtacaacgaagaaaatccgcgcccgggcgttattatcaatagtccgcatggctccgatgtgtatgctggtgttccgaaagattacaccggcgacgaagtcaatgccaaaaattttctggcggccattctgggtaacaaaagcgcaatcaccggcggttctggcaaagtcgtggatagtggtccgaatgaccatattttcatctattacacggatcacggcgcgccgggtgtgattggtatgccgagcaaaccgtatctgtacgcagatgaactgaacgacgctctgcgtaaaaaacacgcgtcaggtacctataaatcgatggtgttttatctggaagcgtgcgaagccggttctatgttcgatggcctgctgccggacggtctgaacatctatgcactgacggcttccaataccacggaaggctcatggtgctattactgtccgggtcaggatgcaggtccgccgccggaatacagcgtgtgtctgggtgactttttctcgattgcctggctggaagatagcgacgtgcataacctgcgttctgaaaccctgaaccagcaataccataacgttaaaaaccgcatctcatatgcgtcgcacgccacgcagtacggcgatctgaaacgcggtgtcgaaggcctgtttctgtatctgggtagtaacccggaaaacgataattacaccttcgtggatgacaacgttgtccgtccgagcagcaaagccgtcaatcaacgcgatgcagacctggtgcacttttgggaaaaattccgtaaagcaccggaaggcagttccaaaaaagaagaagcccagaaacaaattctggaagcaatgtctcatcgcgttcacatcgattcatcgattaatctgatcggcaaactgctgtttggtattgaaaaaggccataaaatcctgaccgccgtgcgtagtgccggtcacccgctggtcgatgactgggcatgcctgcgttccctggtccgtaccttcgaaacgcattgtggcagtctgtcccagtatggtatgaaacacacccgcacgctggcgaacatttgcaatgccggtatcacggaagaacagatggctgaagcagcttcacaagcgtgtgttagcattccgtctaatccgtggagcagccatgatggcggtttttcggcgtga

SEQ ID NO: 41 - protein, 497 aa, artificial
  OaAEP5 aa
  misc_feature (485)..(485): Xaa can be any naturally occurring amino acid
MVRYLAGAFQVVLLVVILSDIAISEERTDGYLKLPTEVSRFFRTPEQSSDGGDDSIGTRWAVLIAGSKGYDNYRHQADVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACEAGSMFEGLLTDDLNIYALTASNATEGSCPYYCPGDLNYSPPPEYDVCLGDFFSIAWLEDSDVHNLRSETLNQQYHNVKNRISYASHATQYGDLKRGVEGLFLYLGSNPENDNYTFVDDNVVRPSSKAVNQRDADLVHFWEKFRKAPEGSSKKEEAQKQILEAMSHRVHIDSSINLIGKLLFGIEKGHKILTAVRSAGHPLVDDWACLRSLVGTFETHCGSLSEYGMRHTRTIANICNAGISEDQMKEAASQACASVPSNSWSSLXKGFHARLAKIIA

SEQ ID NO: 42 - DNA, 1380 bp, artificial
  OaAEP5 na
gaacgcacggatggttatctgaaactgccgacggaagtgagccgcttctttcgcacgccggaacaatcgagcgacggtggtgacgactcaattggtacccgttgggctgtcctgatcgcgggctcgaaaggttatgataactaccgtcatcaggctgacgtgtgccacgcgtatcaaattctgaaacgcggcggtctgaaagatgaaaacatcgtggttttcatgtacgatgacatcgcgtacaacgaatctaatccgcgcccgggcgtgattatcaacagtccgcatggttccgatgtgtatgcgggcgttccgaaagactacacgggtgaagaagttaatgccaaaaattttctggcggccattctgggcaacaaaagtgcaatcaccggcggttccggtaaagtcgtggattcaggcccgaatgaccatattttcatctattacacggatcacggcgcagctggtgtcattggcatgccgagtaaaccgtatctgtacgctgatgaactgaatgacgcgctgaagaaaaaacatgcctcaggtacctataaatcgctggtgttttatctggaagcgtgcgaagccggttccatgttcgaaggcctgctgacggatgacctgaacatctatgcactgaccgcttcgaatgcgacggaaggtagctgcccgtattactgtccgggcgatctgaactatagcccgccgccggaatacgatgtgtgtctgggcgactttttctctattgcgtggctggaagatagtgacgtgcataacctgcgttccgaaaccctgaaccagcaataccataacgttaaaaaccgcatcagctatgcctctcacgcaacgcagtacggtgatctgaaacgtggtgttgaaggcctgtttctgtatctgggcagcaatccggaaaacgataattacaccttcgtcgatgacaacgttgtccgtccgagcagcaaagcagtcaatcagcgcgatgctgacctggtgcacttttgggaaaaattccgtaaagccccggaaggtagttccaaaaaagaagaagcccagaaacaaattctggaagcaatgagccatcgcgtgcacatcgattcatcgattaacctgatcggcaaactgctgtttggtattgaaaaaggccataaaatcctgaccgccgttcgtagcgcaggtcacccgctggtcgatgactgggcatgcctgcgctctctggttggcaccttcgaaacgcattgtggtagtctgtccgaatatggcatgcgtcacacccgcacgattgccaacatctgcaatgcaggtattagtgaagatcagatgaaagaagcggccagccaagcatgtgcttctgtgccgtcaaattcgtggagcagcctgtga

SEQ ID NO: 43 - protein, 489 aa, artificial
  OaAEP6 aa
MVRYLAGAFQVVLLVVILSDIAISEERTDGYLKLPTEVSRFFRTPEQSSDGGDDSIGTRWAVLIAGSKGYANYRHQADVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAPVTIGMPSKPYLYADELIDTLKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHFWDKFRKAPEGSARKEEARKQVFEAMSHRMHIDNSIKLVGKLLFGIERGAEILDAVRPAGQPLADDWTCLKSLVRTFETHCGSLSQYGMKHMRTIANICNAGITKEQMAEASAQACSSVPSNPWSSLHKGFSA

SEQ ID NO: 44 - protein, 495 aa, artificial
  OaAEP7 aa
  misc_feature (483)..(483): Xaa can be any naturally occurring amino acid
MVRYLAGAVLLLVVLSVAAAVSGARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGDDVNAKNFLAAILGNKSAITGGSGKVVDSGPNDHIFIYYTDHGAPVTIGMPSKPYLYADELIDTLKKKHASGGYKSLVFYLEACEAGSMFEGLLTDDLNIYALTASNATEGSCPYYCPGDLNYSPPPEYDVCLGDFFSIAWLEDSDIENSMSETLNQQYHHVKKRIEIASTASQYGNMKLAGEDLFLYIGSNPENDNYTSLHDHALTPSPLAVNQRDADLLHLWEKFRRAPEGSARKEEAQKQIFKTMSDRVHVDNSIKLIGKLLFGIEKGTEILNAVRPAGQPLVDDWACLRSLVGTFERHCGSLSEYGMRHTRTIANICNAGISEDQMKEAASQACASVPSNSWSSLXKGFHARLAKIIA

SEQ ID NO: 45 - protein, 458 aa, artificial
  OaAEP8 aa
MVRYLAGAFQVVLLVVILSDIAISEERTDGYLKLPTEVSRFFRTPEQSSDGGDDSIGTRWAVLIAGSKGYDNYRHQADVCHAYQILKRGGLKDENIVVFMYDDIAYNESNPRPGVIINSPHGSDVYAGVPKDYTGDEVNAKNFLAAILGNKSAITGGSGKVVDSGPNDHIFIYYTDHGAPGVIGMPSKPYLYADELNDALRKKHASGTYKSMVFYLEACEAGSMFDGLLPDGLNIYALTASNTTEGSWCYYCPGQDAGPPPEYSVCLGDFFSIAWLEDSDIENSMSETLNQQYHHVKKRIEIASTASQYGNMKLAGEDLFLYIGSNPENDNYTSLHDHALTPSPLAVNQRDADLLHLWEKFRKAPEGSARKEEAQTQIFKAMSHRVHIDSSIKLIGKLLFGIEKCTEILNAVRPAGQPLVDDWACLRSLVGTFETHCGSLSEYGMRHTRTIANICNAG

SEQ ID NO: 46 - protein, 490 aa, artificial
  OaAEP9 aa
MVRYLAGTVLFLVLLSAAAISEARDGSHLNLPSEVARFFRPQETNDDGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAPGVIGMPSKPYLYADELNDALRKKHASGTYKSMVFYLEACEAGSMFDGLLPDGLNIYALTASNTTEGSWCYYCPGQDAGPPPEYSVCLGDFFSIAWLEDSDVHNLRSETLKQQYHLVKARTSNGNSAYGSHVMQYGDLKLSVDNLFLYMGTNPANDNYTFVDDNALRPSSKAVNQRDADLLHLWEKFRKAPEGSARKEEAQKQIFKTMSDRVHVDNSIKLIGKLLFGIEKGHKILTAVRSAGHPLVDDWACLRSLVRTFETHCGSLSQYGMKHTRTLANICNAGITEEQMAEAASQACVSIPSNPWSSHDGGFSA

SEQ ID NO: 47 - protein, 506 aa, artificial
  OaAEP10 aa
  misc_feature (494)..(494): Xaa can be any naturally occurring amino acid
FSSSCYFQLPETTIMVRYLAGTVLFLVLLSAAAISEARDGSHLNLPSEVARFFRPQETNDDGEDSVGTRWAVLIAGSKGYANYRHQADVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEEAQKQIFKTMSDRVHVDNSIKLIGKLLFGIEKGTEILNAVRPAGQPLVDDWACLRSLVGTFERHCGSLSEYGMRHTRTIANICNAGISEDQMKEAASQACASVPSNSWSSLXKGFHARLAKIIA

SEQ ID NO: 48 - protein, 501 aa, artificial
  OaAEP11
  misc_feature (489)..(489): Xaa can be any naturally occurring amino acid
MVRYLAGAFQVVLLVVILSDIAISEERTDGYLKLPTEVSRFFRTPEQSSDGGDDSIGTRWAVLIAGSKGYANYRHQADVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAPVTIGMPSKPYLYADELIDTLKKKHASGTYKSLVFYLEACESGSMFEGLLPEGLNIYATTASNAEESSWGTYCPGEYPSPPPEYDTCLGDLYSVAWMEDSEVHNLRSETLKQQYHLVKARTSNGNSAYGSHVMQYGDLKLSVDNLFLYMGTNPANDNYTFVDDNALRPSSKAVNQRDADLLHLWEKFRKAPEGSARKEEAQKQIFKTMSDRVHVDNSIKLIGKLLFGIEKGTEILNAVRPAGQPLVDDWACLRSLVGTFERHCGSLSEYGMRHTRTIANICNAGISEDQMKEAASQACASVPSNSWSSLXKGFHARLAKIIA

SEQ ID NO: 49 - protein, 489 aa, artificial
  OaAEP12 aa
MVRYPAGAVLLLVVLSVVAVDGARDGYLKLPSEVSDFFRPRNTNDGDDSVGTRWAVLLAGSNGYWNYRHQADLCHAYQILKRGGLKDENIVVFMYDDIAYNEENPRPGVIINSPHGSDVYAGVPKDYTGDQVNAKNFLAAILGNKSAITGGSGKVVNSGPNDHIFIYYTDHGGPGVLGMPVGPYIYADDLIDTLKKKHASGTYKSLVFYLEACESGSMFEGLLPEGLNIYATTASNAEESSWGTYCPGEYPSPPPEYDTCLGDLYSVAWMEDSEVHNLRSETLKQQYHLVKARTSNGNSAYGSHVMQYGDLKLSVDKLFFYMGTDPANENYTFVDDNDLIRSSSKPVNQRDADLVHFWDKFRKAPEGSARKEEARKQVFEAMSHRMHIDNSIKLVGKLLFGIERGAEILDAVRPAGQPLADDWTCLKSLVRTFETHCGSLSQYGMKHMRTIANICNAGITKEQMAEASAQACSSVPSNPWSSLHKGFSA

SEQ ID NO: 50 - protein, 380 aa, artificial
  OaAEP13 aa
  misc_feature (368)..(368): Xaa can be any naturally occurring amino acid
NPRPGVIFNSPHGSDVYAGVPKDYTGDQVTVKNFLAAILGDKSAITGGSGKVVNSGPNDHIFIYYTDHGGPGVVGMPVGPYLYADDLIDTLKKKHASGTYKSLVFYLEACESGSMFEGLLPEGLNIYATTASNAVEESWATYCPGQHPSAPLEFMTCLGDLFSVAWMEDSEVHNLRSETLNQQYHNVKNRISYASHATQYGDLKRGVEGLFLYLGSNPENDNYTFVDDNALRPSSKAVNQRDADLLHFWDKFRKAPEGSASKEEARKQVFEAMSHRMHIDSSIKLVGKLLFGIEKCTEILNAVRPAGQPLVDDWACLRSLVGTFETHCGSLSEYGMRHTRTIANICNAGISEEQMAEAASQACASIPXNPWSSFHGGFSS

SEQ ID NO: 51 - protein, 506 aa, artificial
  OaAEP14 aa
  misc_feature (494)..(494): Xaa can be any naturally occurring amino acid
MVRYLAGAVLLLVVLSVAAAVSGARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIELIRGSLCYLANLNLRAPSGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEEAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIPXNPWSSFHGGFSS

SEQ ID NO: 52 - protein, 382 aa, artificial
  OaAEP15 aa
NPRPGVIFNSPHGSDVYAGVPKDYTGDQVTVKNFLAAILGDKSAITGGSGKVVNSGPNDHIFIYYTDHGGPGVVGMPVGPYLYADDLIDTLKKKHASGTYKSLVFYLEACESGSMFEGLLPEGLNIYATTASNAVEESWATYCPGQHPSAPLEFMTCLGDLFSVAWMEDSEVHNLRSETLEQQYHQVNAKTRAFGASHVMQYGDLKLSVDNLFLYMGTNPANDNYTFVDDNALRPSSKAVNQRDADLLHFWDKFRKAPEGSASKEEARKQVFEAMSHRMHIDSSIKLVGKLLFGIQRGPEILDAVRPAGQPLADDWSCLKSMVRTFETHCGSLSQYGMKHMRTFANICNAGITKEQMAEASAQACASVPSNPWSSLHRGFSA

SEQ ID NO: 53 - protein, 295 aa, artificial
  OaAEP16 aa
MVRSPAGVVLLLVVLSVVAVSGARDGYLKLPSEVSDFFRPRNTNDGDDSIGTRWAVLLAGSNSYWNYRHQADVCHAYQILKRGGLKDENIVVFMYDDIAYNKYNPRPGVIFNSPHGSDVYAGVPKDYTGDQVTVKNFLAAILGDKSAITGGSGKVVDSGPNDHIFIYYTDHGAPGVIGMPSKPYLYADELNDALRKKHASGTYKSMVFYLEACESGSMFEGILPEDLNIYALTSTNTTESSWCYYCPAQENPPPPEYNVCLGDLFSVAWLEDSDVQNSWYETLNQQYHHVDKRIS

SEQ ID NO: 54 - protein, 487 aa, artificial
  OaAEP17 aa
  misc_feature (475)..(475): Xaa can be any naturally occurring amino acid
MVRYLAGAVLLLVVLSVAAAVSGARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEEAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIPXNPWSSFHGGFSS

SEQ ID NO: 55 - protein, 489 aa, artificial
  Nicotiana tabacum NtAEP1b
MIRYVAGTLFLIGLALNVAVSESRNVLKLPSEVSRFFGADESNAGDHDDDSVGTRWAILLAGSNGYWNYRHQADICHAYQLLKKGGLKDENIVVFMYDDIANNEENPRRGVIINSPHGEDVYKGVPKDYTGDDVTVDNFFAVILGNKTALSGGSGKVVNSGPNDHIFIFYSDHGGPGVLGMPTDPYLYANDLIDVLKKKHASGTYKSLVFYLEACESGSIFEGLLPEGLNIYATTASNAEESSWGTYCPGEYPSPPIEYMTCLGDLYSISWMEDSELHNLRTESLKQQYHLVKERTATGNPVYGSHVMQYGDLHLSKDALYLYMGTNPANDNYTFMDDNSLRVSKAVNQRDADLLHFWHKFRTAPEGSVRKIEAQKQLNEAISHRVHLDNSVALVGKLLFGIEKGPEVLSGVRPAGQPLVDDWDCLKSFVRTFETHCGSLSQYGMKHMRSIANICNAGIKKEQMVEASAQACPSVPSNTWSSLHRGFSA

SEQ ID NO: 56 - protein, 481 aa, artificial
  Petunia hybrida PxAEP3a
MINVAGILILVGFSIIAAGEGRNVLKLPSEASRFFDKGDDDSVGTRWAVLLAGSNGYWNYRHQADVCHAYQLLRKGGLKDENIIVFMYDDIAYNEENPRKGVIINSPAGEDVYKGVPKDYTGDDVNVDNFLAVLLGNKTALTGGSGKVVDSGPNDHIFVFYSDHGGPGVLGMPTNPYLYASDLIGALKKKHASGTYKSLVLYIEACESGSIFEGLLPEGLNVYATTASNAVESSWGTYCPGENPSPPPEYETCLGDLYAVSWMEDSEKHNLQTESLRQQYHLVKRRTANGNSAYGSHVMQFGDLKLSVDSLSMYMGTDPANDNSTFVDDNSLGASSKAVNQRDADLLHFWDKFLKAPEGSARKVEAQKQFTEAMSHRMHLDNSMALVGKLLFGIQKGPEVLKRVRSDGQPLVDDWACLKSFVRTFETHCGSLSQYGMKHMRSIANICNAGIKMEQMVEASSQACPSVPSNTWSSLHRGFSA

SEQ ID NO: 57 - protein, 478 aa, artificial
  Petunia hybrida PxAEP3b
MISHVAGILILVGFSILGAGEGRNVLKLPSEASRFFKKGEDDDSVGTRWAVLLAGSNSYWNYRHQADVCHAYQLLRKGGLKDENIVVLMYDDIAYNEENPRKGVIINNPAGEDVYKGVPKDYTGDDVNVDNFLAVLLGNKTAITGGSGKVVDSGPNDHIFIFYTDHGGPGVLGMPTKPYLYASDLIGALKKKHASGTYKSLVLYVEACEAGSIFEGLLPEGLNVYATTASDAVEGSWVTYCPGQNPSPPPEYTTCLGDLYSVSWMEDSEKHNLQTESLRQQYHLVKEKIAYASHVMQYGDLKLSMDSLSMYMGTDPANDNYTFVDDNSLGTSSKAVNQRDADLLHFSDKFLKAPEGSARKVEAQKQFAEAMSHRLHLDNSMALVGKLLFGIKKGPEVLKRVRSDGQLLVDDWACLKSFVRTFETHCGSLSQYGMKHMRSFANICNAGIKVEQMVEASSQACPSVPSNTWSSLHRGFSA

SEQ ID NO: 58 - protein, 482 aa, artificial
  Clitoria ternatea CtAEP1
MKNPLAILFLIATVVAVVSGIRDDFLRLPSQASKFFQADDNVEGTRWAVLVAGSKGYVNYRHQADVCHAYQILKKGGLKDENIIVFMYDDIAYNESNPHPGVIINHPYGSDVYKGVPKDYVGEDINPPNFYAVLLANKSALTGTGSGKVLDSGPNDHVFIYYTDHGGAGVLGMPSKPYIAASDLNDVLKKKHASGTYKSIVFYVESCESGSMFDGLLPEDHNIYVMGASDTGESSWVTYCPLQHPSPPPEYDVCVGDLFSVAWLEDCDVHNLQTETFQQQYEVVKNKTIVALIEDGTHVVQYGDVGLSKQTLFVYMGTDPANDNNTFTDKNSLGTPRKAVSQRDADLIHYWEKYRRAPEGSSRKAEAKKQLREVMAHRMHIDNSVKHIGKLLFGIEKGHKMLNNVRPAGLPVVDDWDCFKTLIRTFETHCGSLSEYGMKHMRSFANLCNAGIRKEQMAEASAQACVSIPDNPWSSLHAGFSV

SEQ ID NO: 59 - protein, 497 aa, artificial
  Clitoria ternatea CtAEP2
MAVDHCFLKKKTCYYGFVLWSWMLMMSLHSKAARLNPQKEWDSVIRLPTEPVDADTDEVGTRWAVLVAGSNGYENYRHQADVCHAYQLLIKGGLKEENIVVFMYDDIAWHELNPRPGVIINNPRGEDVYAGVPKDYTGEDVTAENLFAVILGDRSKVKGGSGKVINSKPEDRIFIFYSDHGGPGVLGMPNEQILYAMDFIDVLKKKHASGGYREMVIYVEACESGSLFEGIMPKDLNVFVTTASNAQENSWGTYCPGTEPSPPPEYTTCLGDLYSVAWMEDSESHNLRRETVNQQYRSVKERTSNFKDYAMGSHVMQYGDTNITAEKLYLFQGFDPATVNLPPHNGRIEAKMEVVHQRDAELLFMWQMYQRSNHLLGKKTHILKQIAETVKHRNHLDGSVELIGVLLYGPGKGSPVLQSVRDPGLPLVDNWACLKSMVRVFESHCGSLTQYGMKHMRAFANICNSGVSESSMEEACMVACGGHDAGHLHPSKRGYIA

SEQ ID NO: 60 - protein, 46 aa, artificial
  EcAMP1
GLPGSGRGSCRSQCMRRHEDEPWRVQECVSQCRRRRGGGDTRNGLP

SEQ ID NO: 61 - protein, 25 aa, artificial
  R1
GLVFAEFLPLFSKFGSRMHILKGHV

SEQ ID NO: 62 - protein, 25 aa, artificial
  R1
GLVFAEFLPLFSKFGSRMHILKNHV

SEQ ID NO: 63 - protein, 25 aa, artificial
  R1
GLVFAEFLPLFSKFGSRMHILKNHL

SEQ ID NO: 64 - protein, 25 aa, artificial
  R1
GLVFAEFLPLFSKFGSRMHILKNGH

SEQ ID NO: 65 - protein, 25 aa, artificial
  R1
GLVFAEFLPLFSKFGSRMHILKNGF

SEQ ID NO: 66 - protein, 25 aa, artificial
  R1
GLVFAEFLPLFSKFGSRMHILKNFL

SEQ ID NO: 67 - protein, 25 aa, artificial
  R1
GLVFAEFLPLFSKFGSRMHILKDGL

SEQ ID NO: 68 - protein, 25 aa, artificial
  R1
LLVFAEFLPLFSKFGSRMHILKGHV

SEQ ID NO: 69 - protein, 25 aa, artificial
  R1
GKVFAEFLPLFSKFGSRMHILKGHV

SEQ ID NO: 70 - protein, 25 aa, artificial
  R1
GFVFAEFLPLFSKFGSRMHILKGHV

SEQ ID NO: 71 - protein, 16 aa, artificial
  SFT1-110R
MLGRCTKSIPPRCFPD

SEQ ID NO: 72 - protein, 110 aa, artificial
  SFT1-110R
MLGRCTKSIPPRCFPDGLPGGGGSEFELMQIFVKTLTGKTITLEVEPSDTIENVKAKIQDKEGIPPDQQRLIFAGKQLEDGRTLSDYNIQKESTLHLVLRLRGGHHHHHH

SEQ ID NO: 73 - protein, 29 aa, artificial
  Kalata B1
MLPVCGETCVGGTCNTPGCTCSWPVCTRN

SEQ ID NO: 74 - protein, 118 aa, artificial
  Kalata B1
MLPVCGETCVGGTCNTPGCTCSWPVCTRNGLPEFELMQIFVKTLTGKTITLEVEPSDTIENVKAKIQDKEGIPPDQQRLIFAGKQLEDGRTLSDYNIQKESTLHLVLRLRGGHHHHHH

SEQ ID NO: 75 - protein, 24 aa, artificial
  Vc1.1
MLGCCSDPRCNYDHPEICGGAAGN

SEQ ID NO: 76 - protein, 118 aa, artificial
  Vc1.1
MLGCCSDPRCNYDHPEICGGAAGNGLPGGGGSEFELMQIFVKTLTGKTITLEVEPSDTIENVKAKIQDKEGIPPDQQRLIFAGKQLEDGRTLSDYNIQKESTLHLVLRLRGGHHHHHH

SEQ ID NO: 77 - protein, 599 aa, artificial
  Kalata B1 + OaAEP1b
MILHTYIILSLLTIFPKAIGLSLQMPMALEASYASLVEKATLAVGQEIDAIQKGIQQGWLEVETRFPTIVSQLSYSTGPKFAIKKKDATFWDFYVESQELPNYRLRVGLPVCGETCVGGTCNTPGCTCSWPVCTRNGLPAAAGGGGGSARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEVAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIP

SEQ ID NO: 78 - DNA, 1800 bp, artificial
  Kalata B1 + OaAEP1b
atgatcttacacacctatattatcttatccttattaactatcttccctaaagcaatcggtttatccttacaaatgcctatggccttggaagcatcttatgcctcattggttgaaaaagctactttagcagtaggtcaagaaatagatgctatccaaaagggtatccaacaaggttggttggaagtcgaaacaagatttccaaccattgtttctcaattatcctacagtactggtcctaaattcgctattaaaaagaaagatgccacattttgggacttctatgttgaaagtcaagaattgccaaattaccgtttacgtgtcggactccctgtatgtggtgaaacatgcgtcggtggtacttgtaatacaccaggttgtacttgctcatggcctgtttgcacaagaaatggtttgcctgcggccgcgggtggtggtggtggttctgctagagatggtgactatttgcatttgccatccgaagttagtagatttttcagacctcaagaaacaaatgatgaccacggtgaagatagtgtaggtaccagatgggcagtcttgattgccggttccaaaggttatgctaattacagacatcaagccggtgtttgtcacgcttaccaaatattgaagagaggtggtttgaaggatgaaaacatcgttgtttttatgtatgatgacatcgcatacaatgaaagtaacccaagacctggtgtaattataaattccccacatggtagtgatgtctatgccggtgttcctaaagactacactggtgaagaagtcaatgctaagaactttttggctgcaattttaggtaataagtctgcaataacaggtggttcaggtaaagtcgttgatagtggtccaaacgaccatatttttatctattacaccgatcacggtgccgctggtgttattggtatgccatcaaaaccttatttgtacgctgatgaattgaacgacgcattaaagaaaaagcatgcctctggtacatacaagtcattggttttctatttggaagcttgtgaaagtggttcaatgttcgaaggtatcttgccagaagatttgaatatctatgccttaacctcaactaacactacagaaagttcatggtgttattactgccctgctcaagaaaatccacctccacctgaatacaatgtatgcttgggtgacttgttttctgtcgcatggttggaggacagtgatgttcaaaatagttggtatgaaaccttaaaccaacaataccatcatgtagataagagaatatctcatgcctcacacgctactcaatatggtaatttgaagttaggtgaagaaggtttgtttgtttatatgggtagtaacccagctaatgataactacacctctttggacggtaatgcattaactccttccagtattgtagtcaaccaaagagatgctgacttgttacatttgtgggaaaagtttagaaaggcaccagaaggtagtgccagaaaggaagttgctcaaactcaaattttcaaggcaatgtctcatagagtacacatagatagttcaattaaattgatcggtaaattgttgtttggtatagaaaagtgtacagaaatcttgaacgctgtaagaccagcaggtcaacctttagtcgatgactgggcatgtttgagatctttagttggtaccttcgaaactcattgcggttccttaagtgaatatggtatgagacacacaagaaccatcgccaatatttgtaacgctggtatctcagaagaacaaatggcagaagccgcctcccaagcctgtgcatctatcccataa

SEQ ID NO: 79 - protein, 56 aa, artificial
  Target peptides
GLPRECKTESNTFPGICITKPPCRKACISEKFTDGHCSKILRRCLCTKPCTRNGLP

SEQ ID NO: 80 - protein, 7 aa, artificial
  Ligation partner peptide
PLPVSGE

SEQ ID NO: 81 - protein, 60 aa, artificial
  Ligated peptide product
GLPRECKTESNTFPGICITKPPCRKACISEKFTDGHCSKILRRCLCTKPCTRNGLPVSGE

SEQ ID NO: 82 - protein, 60 aa, artificial
  Ligated peptide product
GLPRECKTESNTFPGICITKPPCRKACISEKFTDGHCSKILRRCLCTKPCTRNPLPVSGE

SEQ ID NO: 83 - protein, 25 aa, artificial
  Target peptide
GKVFAEFLPLFSKFGSRMHILKNGL

SEQ ID NO: 84 - protein, 26 aa, artificial
  Ligated peptide product _ C-terminal biotin
GKVFAEFLPLFSKFGSRMHILKNGLK

SEQ ID NO: 85 - protein, 28 aa, artificial
  Ligated peptide product + N-terminal biotin
TRNGLVFAEFLPLFSKFGSRMHILKGHV

SEQ ID NO: 86 - protein, 29 aa, artificial
  R1
GLPVFAEFLPLFSKFGSRMHILKSTRNGL

SEQ ID NO: 87 - protein, 21 aa, artificial
  Bac2A
GLPRLARIVVIRVARTRNGLP

SEQ ID NO: 88 - protein, 31 aa, artificial
  Kalata B1
GLPVCGETCVGGTCNTPGCTCSWPVCTRNGL

SEQ ID NO: 89 - protein, 25 aa, artificial
  R1
LLVFAEFLPLFSKFGSRMHILKNGL

SEQ ID NO: 90 - protein, 25 aa, artificial
  R1
GKVFAEFLPLFSKFGSRMHILKNGL

SEQ ID NO: 91 - protein, 25 aa, artificial
  R1
GFVFAEFLPLFSKFGSRMHILKNGL

SEQ ID NO: 92 - protein, 415 aa, artificial
  Cicer arietinum
MERRMRFWVVALIVKVCMIITMTKSKGQEDYGVQWAFLIAGSKGYRNYRHQADVCHAYQVLRIGGLKDENIIVMMYDDIAYNKENPHPGYIANKPHGINVYFNVPKDYTGKDATKENFYAVLSGKKSGVKGGSGKVLDTNPDDTIFIFFSGHGNTGLIALPDGRTVYADRFINTLKAKINYNKMVIYLESCNAGSMFQGLLPNNLNIYATTASNPFENSYAFYCPKRQSSPPPQYTVCLGNLYSISWLEDSEQNDRESESLNQQYLKVSRSINYRYSHVMQYGNMRMAGDLLFTYLGTNLSPAKDNYHFNTTATHEHSYKPFNMTTSQQDAHLLYLKLKCLWKRHQAQIELDDEISRRKHEDQSVYLIWKILFGEDTRSIMMANLRSDAQPLVDDWNCLRILKKTAAASQVCRVP

SEQ ID NO: 93 - protein, 460 aa, artificial
  Medicago truncatula
MNHKNKYWVALIASIWMSVTDNVFAEGESTTGKKWAFLVAGSNGYVNYRHQADICHAYQILKKGGLKDENIVVFMYDDIAYNPQNPRRGVLINHPNGSDVYNGVPKDYIGDYGNLENFLAVLSGNKSATKGGSGKVLDTGPDDTIFIFYTDHGSPGSIGIPDGGLLYANDFVDALKKKHDAKSYKKMVIYMEACEAGSMFEGLLPNDINIYVTTASNKSENSYGFYCPNSYLPPPPEYDICLGDLYSISWMEDSEKNDMTKEILKEQYETVRQRTLLSHVLQYGDLNISNDTLITYIGADPTNVNDNFNVTSTTNVFSFDDFKSPNPTRNFGQRDAHLIYLKTKLGRASSGSEDKLKAQKELEVEIARRKHVDNNVHQISDLLFGEEKGSIVMVHVRASGQPLVDNWDCLKTLVKTYESHCGTLSSYGRKYLRAFANMCNNGITVKQMVAASLQACLEKN

SEQ ID NO: 94 - protein, 442 aa, artificial
  Hordeum vulgare
MRLQLFAASIALLAVIGTASAGQNWAVLVAGSNGWYNYRHQSDVCHAYQILHKNGIPDSNIIVMMYDDLAKNKQNPTPGIIINHPNGQDVYKGVPHDYTGNTVTPKNFINVLLGKKDAMKGVGSGKVLESGPDDNVFIYFTDHGATGLVAFPTGVLYAKDLNKTIAQMNEEKKYKEMVIYIEACESGSMLEGLLPDNINIYATTASNAEESSYACYYDSKRQTYLGDLYSVNWMEDSDAEDIGKETLFKQFQVTKQKTTESHVMQYGDLNLGAQHTVSEFQGTTRNGKQQSVSPVVDRMNTLLKRETAATVDVRISILSKRLAASPVNSEERLSIERELAHTVRQRTIISSTIDSIAKKSFEVNRSAYADLVTSQRMKLTQHDCYKDATQRIHDKCFDIQNEFVLNKLWIVANLCEVGFHSFTINNAVDAVCGVLGRQQFEY

SEQ ID NO: 95 - protein, 465 aa, artificial
  Gossypium raimondii
MTTLVAGVLLLLLSVTGIVTAQRDATGDVLRLVSPEAYKFFHQSDDGRVGGSRWAVLIAGSRGYENYRHQADVCHAYQLLRKCGLKDENIVVFMYDDIAYNENNPRPGIIINSPNGSDVYHGVPKDYTGDDVTVNNFFNVILGNKAAITGGSGKVVNSGPNDHIFIFYSDHGASGVLGMPDDSYIYANDLNWVLRKKHASGTYKSLVFYIEACESGSIFDGLLDPKGLNIYATTASNATESSWATYCPGGQPSAPPEYDTCLGDLYSVAWIEDSEAHDPRTETLQQQYQNVKKRATTSHVMQYGDIVLSLDHLSVYFGENTAKYNLQPPTTAINQRDADLVHFWEKYRKAPEGSAKKAEAQKQLVEIMSHRMHIDTSVKLIGNLLFGTEIGPDVLNVVRPAGQPLVDDWKCLKEMVKTFETHCGKLAQYGMKYIRSFANICNAGIQIEHMAEASAQACVGIHADH

SEQ ID NO: 96 - protein, 576 aa, artificial
  Chenopodium quinoa
MRKNSCHLMIIQLTVIIFALFFSLSVECRLTSKVFDDLSLDSSNNSDVFLNGGEKWAILIAGSSGYENYRHQADVCHAYQVMKKGGLKDENIIVFMYDDIAFNVDNPNQGVIINRPVGRNVYTNVPKDYTGKNLTTKNFFAAILGYKKAIKGGSGKVLDSGPNDHIFIFYSDHGSAGMLGMPQNEPAIYAKDFIEVLKKKHASNTYKSMVIYLEACESGSIFDGLLPNNLSIYATTASNPDESSYATYCDGDPGVPSEYNNTCLGDLYSISWMEDSERKDPRNETLRQQFAVVKNRTSEMSHVSEYGDVHLSSNYLSLYIALESRKPNQTYSMTNQSEPITPLYVVEQREADLIYFKEMVRRAPEGSKQKIEAQKRLDDVISQRKHVDQTVQAIAKQLFGESRGPSYLTKNRPAGTPLVDDWDCFKAMVSTYEEHCGSLQSYGKKYARAFANFCNAGIHIDRMAQVSAQVCANNENLLARTEEFKVYRGKHYESDADDSPAKNVVVKKWVIRTMNTRITRCFVFVLIIANVYSTVDGILDATVTFIKGNIIPAVIKGVDFVSLIVIRSDRDIGEDV

SEQ ID NO: 97 - protein, 476 aa, artificial
  CtAEP6
MDSFPTLLLFLFLLSLATLVSARHALPGDFLRFPSDQDNLPGTSWAVLLAGSKDYWNYRHQADICHAYQILRKGGLKEENIIVFMYDDIAFNENNPRPGVIINKPDGDDVYEGVPKDYTGEDVNVNNFFAVLLGNKSALTGGSGKVLNSGPNDHIFIFYSDHGGPGVLGMPTHPYLYADDLNEVLKKKHASGTYKRLVFYIEACESGSIFEGLLPEDIDIYATTASNATESSSPTYCPRPPAEHAPFPEYTTCLGDLYSITWMEDSEKHNLQTETLHQQYKLLKERVSLRSNVMQYGDIDISSDVLFQYLGTNPTNENFTFMDENYLRSSSKSINQRDADLIHFWHKFHKALEGSTHKNTAQKQVLEVMSHRMHIDNSVQLIRKLLFSIEKGPETLNKVRPAGSVLVDDWGCLKTMVRTFETHCGSLSQYGMKHMRSFANICNARIKNEQMAKASAQACVSIPTNPWSSLQRGFSA

SEQ ID NO: 98 - protein, 9 aa, artificial
  NaD1
GLPTRNGLP

SEQ ID NO: 99 - protein, 7 aa, artificial
  Ligated peptide
GLPVSGE

SEQ ID NO: 100 - protein, 7 aa, artificial
  Ligated peptide
PLPVSGE

SEQ ID NO: 101 - protein, 25 aa, artificial
  R1 peptide derivative
GKVFAEFLPLFSKFGSRMHILKNGL

SEQ ID NO: 102 - protein, 3 aa, artificial
  Ligation partner
GLK

SEQ ID NO: 103 - protein, 25 aa, artificial
  R1 peptide derivative
GLVFAEFLPLFSKFGSRMHILKGHV

SEQ ID NO: 104 - protein, 5 aa, artificial
  Ligation peptide
TRNGL